(19) United States
(12) Patent Application Publication
(10) Pub. No.: US 2025/0346952 A1
(43) Pub. Date: Nov. 13, 2025

(54) ILLUMINATION SYSTEMS FOR NUCLEIC ACID SEQUENCING

(71) Applicant: Element Biosciences, Inc., San Diego, CA (US)

(72) Inventors: Russell HUDYMA, San Diego, CA (US); Steve Xiangling CHEN, San Diego, CA (US); Arash GHORBANI, San Diego, CA (US); Michael PREVITE, San Diego, CA (US); Yanfei JIANG, San Diego, CA (US); Chaoyi JIN, San Diego, CA (US); Derek FULLER, San Diego, CA (US); Cassandra NIMAN, San Diego, CA (US); Geoffrey PILAND, San Diego, CA (US); Daison RONG, San Diego, CA (US); Gregory GEMMEN, San Diego, CA (US); Jordan NEYSMITH, San Diego, CA (US)

(21) Appl. No.: 19/276,866

(22) Filed: Jul. 22, 2025

Related U.S. Application Data
(63) Continuation of application No. PCT/US2024/012802, filed on Jan. 24, 2024.
(60) Provisional application No. 63/481,583, filed on Jan. 25, 2023.
(60) Provisional application No. 63/489,150, filed on Mar. 8, 2023.

(51) Int. Cl. C12Q 1/6874; G01N 21/64; G02B 21/06; G02B 21/16; G02B 27/14; G02B 27/48

(52) U.S. Cl. CPC C12Q 1/6874; G01N 21/6428; G01N 21/6452; G01N 21/6458; G02B 21/06; G02B 21/16; G02B 27/141; G02B 27/48; G01N 2021/6439; G01N 2021/6463; G01N 2021/6484

(57) ABSTRACT

The present disclosure describes illumination methods and systems for illumination and sequencing applications that can be utilized for, for example, microscopy and sequencing platforms. The methods and systems of the present disclosure can provide wide area, flat illumination, which can reduce error and improve system throughputs.

### CROSS-REFERENCE

This application is a continuation of International Application No. PCT/US2024/012802, filed Jan. 24, 2024, which claims priority to, and the benefit of, U.S. Provisional Application No. 63/481,583, filed Jan. 25, 2023 and U.S. Provisional Application No. 63/489,150, filed Mar. 8, 2023, each of which is incorporated herein by reference in their entirety.

### SUMMARY

Described herein are optical systems of imaging modules for sequencing DNA samples nucleic acids. The optical systems and methods described herein are capable of illuminating multiple surfaces of the flow cells that are axially-displaced from each other with an illumination field with relatively uniform illumination power density. The illumination field filed can be much wider than what some illumination systems are capable of providing, therefore advantageously achieving improved sequencing throughput within a set system run time. The speckle noise of the illumination system can be advantageously reduced using cost-effective and easy-to-implement despecklers. As such, the illumination systems and methods herein can increase effectiveness and efficiency of sequencing analysis including next generation sequencing (NGS).

In some cases, the optical systems and optical assemblies of the present disclosure can provide time sequential color imaging of large areas. Such imaging can enable enhanced sequencing or other imaging performance, where high resolution imaging of a wide area can improve throughputs and reduce the time needed to image a surface. The optical systems and optical assemblies of the present disclosure can provide for reduced volumes of the optical systems and assemblies, which can reduce footprints and enable new system architectures. The number of optical components (e.g., lenses, etc.) can be reduced using the optical systems and assemblies of the present disclosure, reducing the number of elements to be aligned and the number of failure points of the system, enhancing uptime and reducing manufacturing burden. The optical systems and assemblies of the present disclosure may eliminate the movement of a stage in the z direction (e.g., along the optical axis, relative to the optical assembly) of the system, which can provide simpler setups as well as enhanced reliability of the optical system.

Aspects disclosed herein, in some embodiments, provide optical systems, comprising: a stage configured to hold a solid support; a light source configured to illuminate said solid support; and an optical assembly disposed at least partly within an optical path from said stage to said light source, wherein said optical assembly is configured to provide an illumination over an area of said solid support that is greater than about 20 square millimeters (mm²) with a peak-to-valley variation of at most about 5%. In some embodiments, said optical assembly does not comprise an objective. In some embodiments, said optical system does not comprise said objective. In some embodiments, said optical assembly does not comprise a tube lens. In some embodiments, said optical system does not comprise said tube lens. In some embodiments, said stage does not adjust in an optical axis of said system. In some embodiments, said illumination has an irradiance of at least about 40 milliwatts per square millimeter. In some embodiments, said optical assembly is configured to receive an emission light from said solid support. In some embodiments, said optical assembly has a numerical aperture (NA) of at least about 0.3. In some embodiments, said emission light has a wavelength of about 500 nanometers to about 750 nanometers. In some embodiments, said optical assembly has a working distance of at least about 1 mm to 25 mm. In some embodiments, the optical system further comprises a motion coil housed within said optical assembly configured to move a focusing element within said optical path of said optical system. In some embodiments, a motor external to said optical system is configured to move a focusing element along the optical axis in one or both directions. In some embodiments, said motor is coupled directly with a piece of a first, second, or third housing of said optical assembly, and the piece of the first, second, or third housing of said optical assembly is coupled directly with said focusing element. In some embodiments, said light source is a pulsed light source. In some embodiments, said optical system has a composite root mean square error of less than about 0.05. In some embodiments, said optical assembly has an illumination efficiency of at least about 90%. In some embodiments, said area is greater than 30 mm². In some embodiments, said area is greater than 50 mm² or 60 mm². In some embodiments, the optical system further comprises said solid support within said stage. In some embodiments, said solid support comprises two or more surfaces having one or more samples immobilized thereon which are imaged by said optical system. In some embodiments, said solid support comprises three or more surfaces having one or more samples immobilized thereon imaged by said optical system.

In some embodiments, said three or more surfaces are axially displaced from each other at least along an optical axis of said optical system. In some embodiments, said solid support comprises a probe configured to bind a nucleic acid molecule. In some embodiments, said probe is bound to a surface of said solid support. In some embodiments, said light source is a laser light source. In some embodiments, said optical assembly comprises a dichroic filter configured to transmit said illumination. In some embodiments, said optical assembly comprises a first segment comprising a first housing comprising a first plurality of lenses, a second segment comprising a second housing, and a third segment comprising a third housing comprising a second plurality of lenses. In some embodiments, said first segment and said third segment are optically aligned. In some embodiments, said first segment is positioned between said third segment and said stage. In some embodiments, said third segment is positioned between said first segment and an image sensor of the optical system. In some embodiments, said first plurality of lenses are movable along said optical path with a range of about 0 to about 2 millimeters. In some embodiments, said first plurality of lenses comprises an asymmetric convex-convex lens. In some embodiments, said second plurality of lenses comprises an asymmetric concave-concave lens. In some embodiments, said asymmetric concave-concave lens is an aspheric asymmetric concave-concave lens. In some embodiments, said optical system is configured to acquire images of said solid support without moving an optical compensator into the optical path between said solid support and a detector of the optical system. In some embodiments, said optical system is configured to acquire images of said solid support without moving an optical compensator out from the optical path between the sample and a detector of the optical system. In some embodiments, said solid support is a flow cell. In some embodiments, said optical assembly is configured to generate one or more spatial constrictions lateral to said optical path of light which travels therethrough. In some embodiments, said optical assembly is configured to generate one or more field curvature corrections lateral to said optical path of light which travels therethrough. In some embodiments, said optical assembly is configured to generate at least one field curvature correction lateral to the optical path of light travels therethrough in a first segment, second segment, or third segment.

In another aspect, the present disclosure provides a method of analyzing a biological molecule, comprising: (a) providing a solid support comprising said biological molecule comprising a label; (b) using an optical system comprising a light source to provide illumination to said biological molecule comprising said label, thereby generating a signal light or a change thereof, wherein said illumination is provided over an area of said solid support that is greater than about 20 square millimeters (mm²) with a peak-to-valley variation of at most about 5%; (c) detecting, using a detector of said optical system, said signal light or said change thereof; and (d) processing at least in part said signal light or said change thereof to analyze said biological molecule. In some embodiments, said biological molecule is a nucleic acid molecule, a protein, or a polypeptide. In some embodiments, said biological molecule is a nucleic acid. In some embodiments, the method further comprises, prior to (a), binding said biological molecule to a probe bound to said solid support, and coupling said label to said biological molecule. In some embodiments, said label is coupled to said biological molecule by hybridization. In some embodiments, said optical system does not comprise an objective. In some embodiments, said solid support is not moved in an optical axis of said optical system. In some embodiments, a plurality of images of said solid support are acquired without moving said solid support in said optical axis. In some embodiments, said illumination has an irradiance of at least about 40 milliwatts per square millimeter. In some embodiments, said signal light has a wavelength of about 500 nanometers to about 750 nanometers.

In some embodiments, said detecting of (c) is performed using an optical element with a numerical aperture of at least about 0.3. In some embodiments, the method further comprises, in (b), using a motion coil within said optical system to move a focusing element within an optical path of said optical system, thereby changing a focus of said optical system on said solid support. In some embodiments, said light source is a pulsed light source. In some embodiments, said illumination is provided with an efficiency of at least about 90%. In some embodiments, the method further comprises repeating (b)-(d) for an additional biological molecule coupled to an additional surface of said solid support. In some embodiments, the method further comprises, subsequent to (c), removing said label from said biological molecule. In some embodiments, the method further comprises repeating (a)-(d) for an additional label that binds to another portion of the biological molecule. In some embodiments, an optical assembly is configured to generate one or more spatial constrictions lateral to said optical path of light which travels therethrough. In some embodiments, an optical assembly is configured to generate one or more field curvature corrections lateral to said optical path of light which travels therethrough. In some embodiments, an optical assembly is configured to generate at least one field curvature correction lateral to the optical path of light that travels therethrough in a first segment, second segment, or third segment. In some embodiments, (d) comprises processing, at least in part, said signal light or said change thereof to generate one or more solid support images and analyze said one more solid support images to generate base calls of the sample. In some embodiments, each of said solid support images comprises a field-of-view (FOV) that is greater than 20 square millimeters (mm²). In some embodiments, said solid support is a flow cell.

Aspects disclosed herein, in some embodiments, provide optical systems, comprising: a stage configured to hold a solid support; a light source configured to illuminate said solid support; and a despeckler optically coupled to said light source and disposed within an optical path from said light source to said stage. In some embodiments, the despeckler is configured to reduce speckle noise introduced between the light source and the stage. In some embodiments, the optical system further comprises an additional light source optically coupled into said despeckler. In some embodiments, light from said additional light source is configured to illuminate said solid support with a different wavelength of light from said light source. In some embodiments, at least about 4 light sources are coupled into said despeckler. In some embodiments, said despeckler is a vibrational despeckler. In some embodiments, said despeckler is a passive despeckler. In some embodiments, said passive despeckler comprises a diffuse scattering plate. In some embodiments, said despeckler is a tension despeckler. In some embodiments, said despeckler is configured to reduce speckle noise to at most about 5%. In some embodiments, said solid support is a flow cell.

Aspects disclosed herein, in some embodiments, provide methods for analyzing a biological molecule, comprising: (a) providing a solid support comprising a biological sample comprising a label; (b) using an optical system comprising a light source to provide illumination to said biological sample comprising said label, thereby generating a signal light or a change thereof, wherein said illumination is provided through a despeckler in an optical path of said optical system; (c) detecting, using a detector of said optical system, said signal light or said change thereof, and (d) processing at least in part said signal light or said change thereof to analyze said biological molecule.

In some embodiments, the method further comprises repeating (b)-(d) for an additional biological sample coupled to an additional surface of said solid support. In some embodiments, the method further comprises, subsequent to (c), removing said label from said biological sample. In some embodiments, the method further comprises repeating (a)-(d) for an additional label that binds to said biological sample. In some embodiments, said despeckler uses vibration to despeckle said illumination. In some embodiments, the method further comprises using an additional light source to illuminate said solid support. In some embodiments, said additional light source provides a different wavelength of light to said solid support. In some embodiments, said additional light source is optically coupled to said despeckler. In some embodiments, said biological sample comprises a nucleic acid molecule, a protein, or a polypeptide. In some embodiments, said biological sample comprises a nucleic acid. In some embodiments, the optical assembly is disposed at least partly within an optical path from said stage to a detector of the optical system. In some embodiments, an illumination system of the optical assembly is disposed within an optical path from said stage to a detector of the optical system. In another aspect, the present disclosure provides a sample stage for holding DNA samples for DNA sequencing reactions and imaging, comprising: a base stage comprising a top surface, wherein the base stage is rotatable about a z-axis relative to an optical system of a sequencing system; one or more top stages positioned on the top surface of the base stage, wherein each of the one or more top stages are configured to receive and secure one or more flow cell devices thereon, and wherein said each of the one or more top stages are movable relative to the base stage; a first motor configured to actuate the base stage to rotate with a first resolution. In some embodiments, the top surface is of a circular shape. In some embodiments, the first resolution is angular resolution and less than 0.1 degrees, 0.2 degrees 0.5 degrees, 1 degree, 2 degrees, 3 degrees, 4 degrees, 5 degrees, 10 degrees, 20 degrees, 30 degrees, or 50 degrees. In some embodiments, each of the flow cell devices comprises one or more samples immobilized thereon to be sequenced. In some embodiments, at least one of the flow cell devices comprises an in situ sample immobilized thereon. In some embodiments, the sample stage further comprises one or more second motors configured to actuate the one or more top stages relative to the base stage at a second resolution individually. In some embodiments, the sample stage further comprises a second motor configured to acuate the one or more top stages relative to the base stage at a second resolution simultaneously. In some embodiments, the second resolution is less than 0.01 mm, 0.015 mm, 0.02 mm, 0.03 mm, 0.04 mm, 0.05 mm, 0.08 mm, 0.1 mm, 0.2 mm, or 1 mm. In some embodiments, the sequencing system comprises a fluidic control device in fluidic communication with the flow cell devices positioned on the sample stage. In some embodiments, said each of the one or more top stages are movable within a sample plane relative to the base stage. In some embodiments, a first top stage of the one or more top stages is movable independently relative to a second top stage of the one or more top stages. In some embodiments, a first top stage of the one or more top stages is movable simultaneously with a second top stage of the one or more top stages relative to the base stage. In some embodiments, said each of the one or more top stages are movable along a radius of the top surface of the base stage relative to the base stage. In some embodiments, said each of the one or more top stages are movable orthogonal to a radius of the top surface of the base stage relative to the base stage.

Aspects disclosed herein, in some embodiment, provide methods of sequencing multiple DNA samples positioned on a rotary sample stage, comprising: obtaining a sample stage comprising a base stage and one or more top stages positioned on a top surface of the base stage, wherein the base stage is rotatable about a z-axis relative to an optical system of a sequencing system; positioning and securing a first flow cell device relative to a first top stage of the one or more top stages; positioning and securing a second flow cell device relative to a second top stage of the one or more top stages; dispensing, by a first fluidic control device, one or more sequencing reagents to the first flow cell device; imaging a first sample region of the first flow cell device using the optical system of the sequencing system; moving the first top stage within the x-y plane relative to the optical system while preventing the second flow cell device from moving relative to the optical system; imaging a second sample region of the first flow cell device using the optical system of the sequencing system; rotating the sample stage with a predetermined angular resolution to position the second flow cell device in a predetermined position relative to the optical system; and imaging a first sample region of the second flow cell device using the optical system of the sequencing system.

In some embodiments, moving the first top stage within the x-y plane relative to the optical system while preventing the second flow cell device from moving relative to the optical system comprises: moving the first top stage along a radius of the top surface of the base stage with a predetermined distance relative to the optical system independently while preventing the second flow cell device from moving relative to the optical system. In some embodiments, moving the first top stage within the x-y plane relative to the optical system while preventing the second flow cell device from moving relative to the optical system comprises: moving the first top stage along a direction orthogonal to a radius of the top surface of the base stage with a predetermined distance relative to the optical system independently while preventing the second flow cell device from moving relative to the optical system. In some embodiments, the method further comprises: moving the first fluidic control device or a second fluidic control device to position the second fluidic cell device in a predetermined position relative to the first fluidic control device or the second fluidic control device. In some embodiments, the first sample region or the second sample region comprises a tile.

In some embodiments, each of the one or more top stages comprises a motion range of greater than 15 mm and less than 80 mm, along a radius or orthogonal to the radius of the top surface of the base stage. In some embodiments, each of the one or more top stages comprises a motion range of greater than 25 mm and less than 100 mm, along a radius or orthogonal to the radius of the top surface of the base stage.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings of which:

FIG. 1 shows a non-limiting example of the illumination system of the optical assembly herein, which includes an illumination subsystem and a light beam delivery subsystem.

FIG. 2 shows a non-limiting example of the illumination subsystem herein.

FIGS. 3A-3C show uniformity in the illumination power density by the illumination system of FIG. 1 herein. FIG. 3A shows an example image of an illumination field and the associated illumination intensity. FIG. 3B shows a line trace of the illumination intensity along the long axis of FIG. 3A. FIG. 3C shows a line trace of the illumination intensity along the short axis of FIG. 3A.

FIG. 4 illustrates a non-limiting example of the illumination subsystem and the light beam delivery system of the optical assembly.

FIG. 5 illustrates a non-limiting example of the illumination subsystem of the optical assembly.

FIG. 6 shows a despeckler and its relative position to a collimator of the light beam delivery subsystem.

FIG. 7 shows a non-limiting example of the optical fiber and the light beam delivery subsystem.

FIG. 8 shows a non-limiting example of the liquid light guide and the light beam delivery subsystem.

FIGS. 9A-9D show non-limiting examples of the despeckler, in this case, a mechanical vibration source that is loosely or fixedly attached to at least a portion of the optical fiber(s). FIG. 9A shows a wound portion of an optical fiber, according to some embodiments. FIG. 9B shows a portion of an optical fiber round around a vibration source, according to some embodiments. FIG. 9C shows a portion of an optical fiber round around a fan vibration source, according to some embodiments. FIG. 9D shows a portion of an optical fiber round around a fan vibration source, according to some embodiments.

FIG. 10 shows a table of different despeckler configurations in relation to the optical fiber and their corresponding speckle noise levels.

FIG. 11 illustrates a block diagram of a sequencing system for imaging DNA sample(s) during DNA sequencing reactions, according to some embodiments.

FIG. 12 is a schematic of various examples of configurations of multivalent molecules. Left (Class I): schematics of multivalent molecules having a “starburst” or “helter-skelter” configuration. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide units are designated ‘N’, biotin is designated ‘B’, and streptavidin is designated ‘SA’.

FIG. 13 is a schematic of an example of a multivalent molecule comprising a generic core attached to a plurality of nucleotide-arms.

FIG. 14 is a schematic of an example of a multivalent molecule comprising a dendrimer core attached to a plurality of nucleotide-arms.

FIG. 15 shows a schematic of an example of a multivalent molecule comprising a core attached to a plurality of nucleotide-arms, where the nucleotide arms comprise biotin, a spacer, a linker and a nucleotide unit.

FIG. 16 is a schematic of an example of a nucleotide-arm comprising a core attachment moiety, a spacer, a linker and a nucleotide unit.

FIG. 17 shows the chemical structure of an example of a spacer (top), and the chemical structures of various examples of linkers, including an 11-atom linker, 16-atom linker, 23-atom linker and an N3 linker (bottom).

FIG. 18 shows the chemical structures of various examples of linkers, including linkers 1-9.

FIG. 19 shows the chemical structures of various examples of linkers joined/attached to nucleotide units.

FIG. 20 shows the chemical structures of various examples of linkers joined/attached to nucleotide units.

FIG. 21 shows the chemical structures of various examples of linkers joined/attached to nucleotide units.

FIG. 22 shows the chemical structures of various examples of linkers joined/attached to nucleotide units.

FIG. 23 shows the chemical structure of an example of a biotinylated nucleotide-arm. In this example, the nucleotide unit is connected to the linker via a propargyl amine attachment at the 5 position of a pyrimidine base or the 7 position of a purine base.

FIG. 24 shows a flow chart of a method of analyzing a biological molecule, according to some embodiments.

FIG. 25 shows a flow chart of a method for analyzing a biological sample, according to some embodiments.

FIG. 26 illustrates a perspective view of a non-limiting example of an imaging module or optical assembly.

FIG. 27 illustrates a cross-sectional view of the non-limiting example of an imaging module or optical assembly.

FIG. 28 shows a cross-sectional view of the non-limiting example of the single channel time-sequential color imaging module in FIGS. 26-27.

FIGS. 29A-29B show examples of an external actuator coupling, according to some embodiments. FIG. 29A shows a detail view of an external actuator and optical assembly, according to some embodiments. FIG. 29B shows a far view of an external actuator and the optical assembly, according to some embodiments.

FIG. 30 shows an example of the optical elements of an optical assembly and the associated focus paths, according to some embodiments.

FIG. 31 shows an example of the optical elements of an optical assembly and the associated focus paths, according to some embodiments.

FIGS. 32A-32B provide diffraction modulation transfer functions (MTFs) for optical systems, according to some embodiments. FIG. 32A shows an example MTF for an objective based optical system. FIG. 32B shows an example MTF for an optical system not comprising an objective.

FIGS. 33A-33B show wavefront analysis calculations for an optical system of the present disclosure, according to some embodiments. FIG. 33A shows an example wavefront analysis calculation at position 1. FIG. 33B shows an example wavefront analysis calculation at position 2.

FIG. 34 shows a top surface optical performance curve, according to some embodiments.

FIG. 35 shows a bottom surface optical performance curve, according to some embodiments.

FIG. 36 shows a plot of an MTF of an optical system, according to some embodiments.

FIG. 37 shows a plot of a cumulative probability of achieving a given wavefront error, according to some embodiments.

FIG. 38 is a schematical illustration of a rotatory stage for moving the sample(s) relative to the objective lens of the optical system for imaging sequencing reactions.

### DETAILED DESCRIPTION

There is a need for increased throughput and flexibility in next generation sequencing (NGS) analysis systems. Disclosed herein are optical systems, designs, and methods of using thereof that may provide any one or more of the following advantages: wide field-of-view with uniformity in illumination power, reduction of speckle noise with cost-effective and easy-to-implement methods, higher system throughput for fluorescence imaging-based genomics applications, compatibility with traditional flow cell devices and/or optical systems, flexibility in analysis or comparison of samples (e.g., larger sample volume and/or increased sample variety), reduction in system volume, reduced complexity and other requirements in optical element (e.g., simpler optical setups), larger field-of-view, and improved uniformity in illumination power.

Disclosed herein are imaging modules that are configured for multi-channel florescence imaging. Each optical system may include multiple imaging modules or equivalently, multiple optical assemblies, e.g., an imaging module for each color channel, and one or more imaging modules may include an illumination system disclosed herein and an image acquisition system configured for acquiring flow cell images of a sample or samples immobilized on the sample stage and positioned at a sample plane. The illumination system and/or the image acquisition system may either work individually for each corresponding imaging module or may be shared among multiple imaging modules. As disclosed herein, the imaging module is used interchangeably as optical assembly, according to some embodiments.

### Image Acquisition Systems and Methods

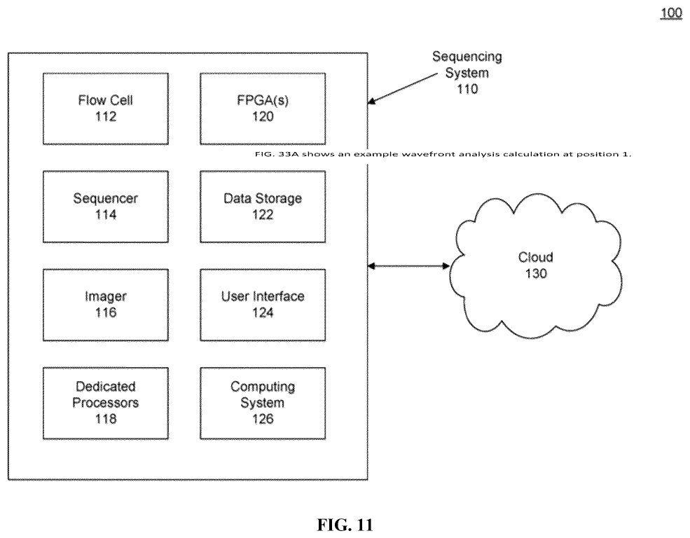

In some embodiments, the image acquisition system comprises one or more image sensors and one or more objective lenses. In some embodiments, the optical system for imaging next-generation sequencing (NGS) reactions, e.g. imager 116 in FIG. 11, may include one or more multi-channel fluorescence imaging modules, each imaging module corresponding to a different color channel. Each imaging module may include an image acquisition system having a corresponding image sensor for such color channel and an objective lens. The objective lens may be shared between more than one imaging module. In some embodiments, each imaging module may include its own sensor and may lack any objective lens. In some embodiments, each imaging module is configured to generate the flow cell images without using any objective lenses.

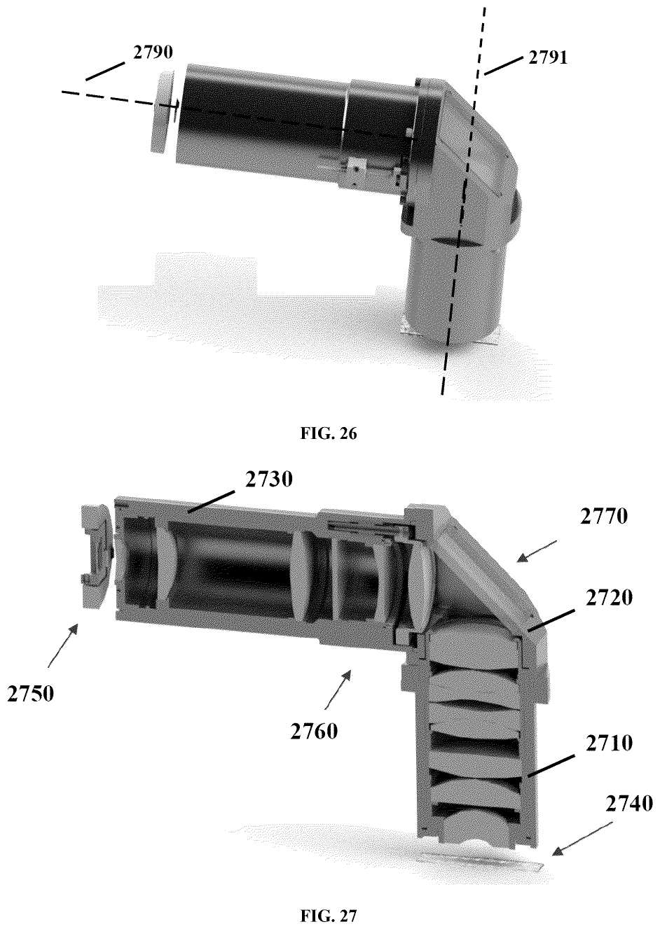

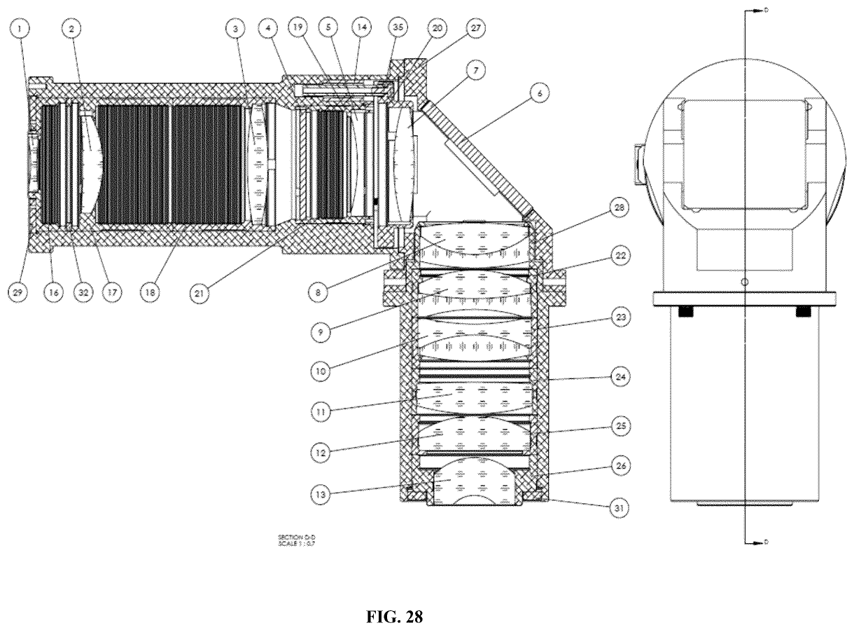

In some embodiments, the imaging module includes 3 different segments. The segments can be optically aligned independently from one another, and can be coupled together to form the imaging module. Having multiple segments may advantageously allow each segment to be independently manufactured and optically aligned. Each segment may include its individual housing. Alternatively, the imaging module can have a housing that houses all three different segments. In some embodiments, the first segment houses a first group of lens elements therein. Some of the lens elements may be movable relative to the housing. For example, 1-3 and 5-8 in FIG. 28 are lens elements housed in the first segment. In some embodiments, the third segment section houses a second group of lens elements, G2. For example, 9-13 in FIG. 28 are in the third segment. Various methods can be used to control centration and angular alignment among multiple segments or of optical elements within a single segment, e.g., an alignment turning technology may be used for the control and angular alignment can be sub-cell based, utilizing alignment turning technology to control centration and angular alignment. The second segment may house the excitation dichroic beam splitter, e.g., 2770 as shown in FIGS. 26-27. In some embodiments, the two orthogonal lens groups may be actively aligned at a nominal 45° angle, which may control the pointing differences between the first lens element G1 and the second lens element Group G2. In some embodiments, active alignment of the two orthogonal lens groups may reduce alignment error(s) to be within a satisfactory range. The satisfactory range can be customized based on different applications. For example, the alignment error can include one or more of: decenter, tilt, lens interval error, and defocus is required to fulfill the error budget.

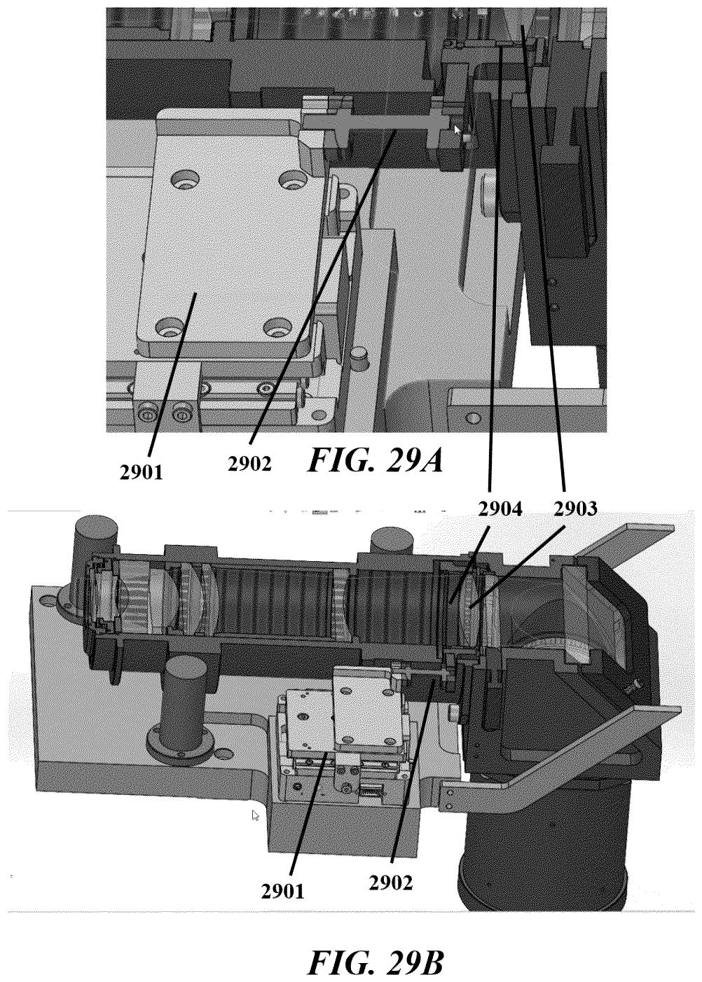

As disclosed herein, the focusing of the imaging module is advantageously internalized. Instead of moving multiple pieces of the lens element, e.g., the entire objective lens, one or more optical compensators, relative to the sample for focusing, the imaging module enables movement of an individual lens element or elements therewithin relative to the housing of the imaging module in order to achieve focusing at least along the z-axis. In some embodiments, a single lens element may be moved relative to the housing to achieve focusing along the z-axis. In some embodiments, two lens elements may be moved together or separately relative to the housing to achieve focusing along the z-axis. As shown in FIGS. 29A-29B a lens element may ride on linear bearings driven by an external actuator so that the lens element can move a predetermined distance automatically in a controlled fashion. The lens element may be movable along the optical axis of the optical assembly. The optical axis, e.g., 2790 in FIG. 26A, of the optical assembly between the detector and the stage may be along the z axis for the segment, e.g., first segment, that is closest to the sample stage. The optical axis of the optical assembly may be along an axis orthogonal to the z axis for the segment that is closest to the image sensor, e.g., the third segment. The optical axis, e.g., 2791 in FIG. 26A, of the optical assembly between the light source and the stage may be along the z axis for the segment, e.g., first segment, that is closest to the sample stage. The optical axis of the optical assembly may be along an axis orthogonal to the z axis for the segment that is closest to the image sensor, e.g., the third segment.

In some embodiments, motion range of the lens element to achieve focusing along the z-axis may be customized based on size and dimension of the flow cell(s). For example, the z-motion range to image the top surface of the flow cell to the bottom surface of the flow cell can be about 810 um, with a lens element moving toward or away from the imaging sensor. In some embodiments, the housing may include hard travel stops which limit the travel range of the lens element during focusing. For example, 8 in FIG. 28 is an element that can be moved for focusing the imaging module. As another example, 5 in FIG. 28 may be an element that can be moved for focusing the imaging module. The travel range may be sufficient to allow focusing of multiple surfaces without interferences with other lens elements, e.g., touching other lens elements. For example, the lens element for focusing may move about 0.1 to 5 mm toward the sensor and about 0.1 to 4.0 mm away from the sensor. In some embodiments, the travel range may be included to help cope with deconjugation or placement error of the surfaces, e.g., the top surface, of the flow cell relative to the vertex of the lens element(s). In some embodiments, the lens element actively aligns E7 to E1-E6. Sample stages and methods of use

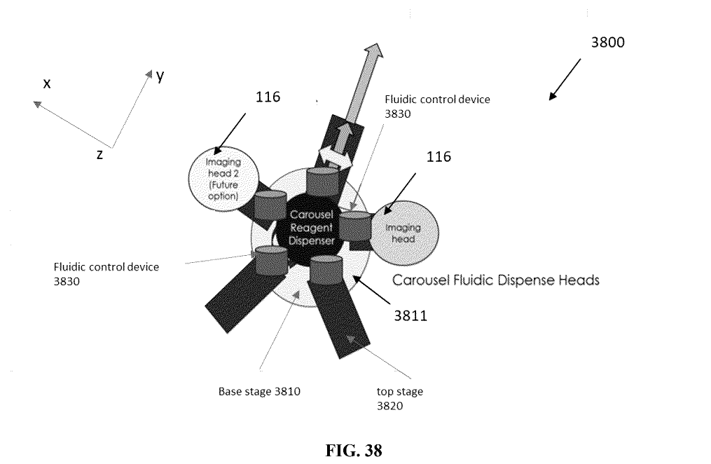

In some embodiments, the optical systems herein (e.g., those used for imaging a sample or sequencing reactions) may include a sample stage configured for holding sample(s) and/or their corresponding support, e.g., a flow cell device with solid support(s), in a prespecified position relative to the optical system. In some embodiments, the sample stage may include a base stage and one or more top stages positioned thereon. FIG. 38 shows an exemplary sample stage 3800 with a base stage 3810 and top stages 3820.

The base stage, e.g., 3810 in FIG. 38, may include a thickness along z axis and a top surface. The thickness of the base stage can be customized to various numbers, e.g. in a range from 1 mm to 5 cm. The top stage(s), e.g., 3820, may be positioned on the top surface 3811 of the base stage. The top surface may be planar. The top surface may be of various geometrical shapes. In some embodiments, the top surface of the base stage may be, but is not limited to, a circular shape, a donut shape, an oval, a square, a rectangle, or a diamond shape. The top surface of the base stage may include a size sufficient to position one or more top stages thereon for sequencing purposes. For example, the top surface may be sufficient to position 5, 10, 20, 30 or even more top stages on it.

The base stage may be configured to move relative to the optical system (116), e.g., relative to the focal plane of the objective lens or the focal plane of the optical system herein to allow focusing of the sample(s) positioned on the base stage for imaging. The base stage may be configured to move in one or more directions in the 3D space. For example, the base stage may be configured to move along x, y, and/or z axis, relative to the focal plane of the optical system. As another example, the base stage may be configured to rotate about an axis, e.g., the z axis, in order to focus different areas of the top surface of the base stage, thus the sample(s) positioned thereon, relative to the focal plane of the optical assembly.

In some embodiments, the sample stage may be of various geometrical shapes. In some embodiments, the base stage may be movable relative to the optical axis of the optical system. In some embodiments, the base stage may be rotatable about the optical axis or z-axis of the optical system.

The one or more top stages can be of various geometrical shapes. For example, as shown in FIG. 38, the 5 top stages are rectangular. In some embodiments, the top stage may be, but is not limited to, a circular shape, a donut shape, an oval, a square, a rectangle, or a diamond shape. In some embodiments, each top stage may have a shape and size that is sufficient to hold one or more flow cell devices thereon.

In some embodiments, the one or more top stages are movable along a radius of the top surface (of the base stage relative to the base stage, e.g., along the y axis as shown in FIG. 38. In some embodiments, the one or more top stages are movable orthogonal to a radius of the top surface of the base stage relative to the base stage, e.g., along the x axis shown in FIG. 38. In some embodiments, the one or more top stages are movable in various directions in the x-y plane.

In some embodiments, a first top stage of the one or more top stages is movable independently relative to at least a second top stage of the one or more top stages. In some embodiments, a first top stage of the one or more top stages is movable simultaneously with at least a second top stage of the one or more top stages relative to the base stage.

In some embodiments, each top stage may have one or more flow cell devices (not shown) immobilized thereon. In some embodiments, the flow cell devices may be removably secured to the corresponding top stage. In some embodiments, movement of the top stage may cause identical movement in the one or more flow cell devices immobilized thereon. The flow cell devices may be secured relative to the top stage so that there is no relative movement between the flow cell device and the corresponding top stage when the top stage moves. The flow cell device may be secured via various secure or fastening means including but not limited to mechanical clamping the flow cell device down, fastening with a magnetic or electro-magnetic force, positioning the flow cell device into a fitted housing which is fastened to the top stage, coupling a pin or post of the top stage to a hole or a grove of the flow device or vice versa.

In some embodiments, each flow cell device may have sample(s) immobilized thereon. The sample(s) can be 2D DNA sample(s). The sample(s) can be 3D volumetric samples of in situ cell(s) and/or tissue. In some embodiments, the sample(s) may be multiplexed samples. In some embodiments, the sample(s) may be of balanced or unbalanced nucleotide diversity.

FIG. 38 shows a non-limiting example of the sample stage 3800 for holding samples that are imaged by the optical system 116 of the sequencing system 110 disclosed herein. In this particular embodiment, the base stage 3810 of the sample stage has a top surface 3811 that is of a circular shape. The top surface may include one or more top stages 3820 coupled thereon. In this embodiment in FIG. 38, there are 5 top stages spaced evenly on the top surface of the base stage. In some embodiments, there can be 1-30 top stages positioned on the sample stage. In some embodiments, the sample stage, e.g., the base stage and the top stages may be rotatable about the optical axis, e.g., z axis. When the base stage rotates, the top stage secured thereon may also rotate together with the base stage in the identical rotatory motion.

In some embodiments, the top stages may be movable relative to the base stage. Such movement may occur separately or simultaneously as the rotating motion of the base stage and the top stages. For example, the rotation of the base stage relative to the optical system, and linear movement of the top stage(s) relative to the base stage can occur simultaneously to position the predetermined sample area of a flow cell device relative to the optical system for imaging efficiently. As another example, the rotation of the base stage relative to the optical system, and linear movement of the top stage(s) relative to the base stage can occur sequentially and it can be controlled by the same motor. The movement of the top stage relative to the base stage may occur in the sample plane, e.g., the x-y plane, that is orthogonal to the z-axis. For each top stage, the x-axis may extend axially from the center of the top surface of the base stage, and the y-axis may be orthogonal to the x and z axis. axes. For example, the top stage at the top of FIG. 38 may move along the y and/or x-axis relative to the sample stage, so that different areas of the top stage may be moved to a specified location relative to the optical system, e.g. the objective lens, for imaging. In some embodiments, the y axis and x axis corresponding to different top stages may change direction within the x-y plane so that the y axis is along the longest dimension of flow cell devices (e.g., along a radius of the top surface of the base stage) and the x axis is along the lateral direction of the flow cell devices (e.g., along tangential direction of the top surface of the base stage).

Each top stage may be configured to hold sample(s) and their corresponding support(s) thereon. The sample(s) and their corresponding support(s) may be immobilized on the stage to move along with the top stage. By having various samples immobilized on different top stages, different samples may be imaged in a time-sequential fashion by rotating the particular sample region via rotation of the base stage, and/or by linearly moving the particular sample region via linear movement of the top stage so that the sample region can be placed into position relative to the optical system for imaging.

In some embodiments, the top stage may include a motion range in the x-y plane sufficient for imaging a pre-determined area of the sample(s). In some embodiments, the motion range along x-axis may be 0 to 50 mm, 0 to 40 mm, 0 to 30 mm, or 0 to 20 mm. In some embodiments, the motion range along y-axis may be 0 to 50 mm, 0 to 40 mm, 0 to 30 mm, 0 to 20 mm, 0 to 16 mm, or 0 to 10 mm. The resolution of movement along x or y axis can be customized based on different sample(s) or sequencing applications. In some embodiments, resolution of movement along x or y axis can be from 1 μm to 40 μm, from 1 um to 30 μm, from 1 μm to 20 μm, or from 1 μm to 10 um.

In some embodiments, each of the one or more top stages comprises a motion range of greater than 15 mm and less than 80 mm, along a radius or orthogonal to the radius of the top surface of the base stage. In some embodiments, each of the one or more top stages comprises a motion range of greater than 25 mm and less than 100 mm, along a radius or orthogonal to the radius of the top surface of the base stage.

The optical system may include one or more imaging head(s), e.g., one or more optical assemblies disclosed herein. Two imaging heads are shown in FIG. 38. Having one imaging head may advantageously decrease the cost of the optical systems, volume of the system, and system complexity, while having more imaging heads may advantageously increase imaging throughput and reduce total imaging time for imaging a certain number of samples, with the trade-off of increased system hardware cost, complexity, etc. In some embodiments, the sample stage further comprises a first motor configured to actuate the sample stage, e.g., the base stage and the top stages, to rotate with a first resolution. The rotation of the sample stage can be relative to the optical system, e.g., the optical axis of the optical system. The first resolution may be an angular resolution that is less than 0.1 degrees, 0.2 degrees 0.5 degrees, 1 degree, 2 degrees, 3 degrees, 4 degrees, 5 degrees, 10 degrees, 20 degrees, 30 degrees, or 50 degrees. The first resolution may be an angular resolution that is greater than 0.1 degrees, 0.2 degrees 0.5 degrees, 1 degree, 2 degrees, 3 degrees, 4 degrees, 5 degrees, 10 degrees, 20 degrees, 30 degrees, or 50 degrees. In some embodiments, various actuation mechanisms may be used to enable rotating motion of the sample stage. For example, a geared mechanism or an induction-based motor may be used to actuate the motion of the sample stage. In some embodiments, the resolution of rotating motion can be customized. For example, the resolution may be 0.1, 0.2, 0.5 degrees. In some embodiments, the sample stage may rotate a minimum of 10 to 360 degrees. In some embodiments, the sample stage may rotate in any number of full circles. In some embodiments, the sample stage may rotate in one or both directions.

In some embodiments, the same mechanism for the base stage or a different actuation mechanism may be used to acuate the top stage(s) for their movements. In some embodiments, the sample stage further comprises one or more second motors configured to acuate some of the one or more top stages relative to the base stage at a second resolution independently without moving the rest of the top stages. In some embodiments, the sample stage further comprises a second motor configured to acuate the one or more top stages relative to the base stage at a second resolution simultaneously. In some embodiments, the second resolution is less than 0.01 mm, 0.015 mm, 0.02 mm, 0.025 mm, 0.03 mm, 0.05 mm, 0.08 mm, 0.1 mm, 0.15 mm, 0.2 mm, or 0.5 mm. In some embodiments, the second resolution is less than 0.01 mm, 0.02 mm, 0.05 mm, 0.1 mm, 0.2 mm, 0.4 mm, 0.8 mm, 1 mm, 2 mm, or 5 mm. In some embodiments, the second resolution is greater than 0.01 mm, 0.02 mm, 0.05 mm, 0.1 mm, 0.2 mm, 0.4 mm, 0.8 mm, 1 mm, 2 mm, or 5 mm. In some embodiments, the second resolution is greater than 0.01 mm, 0.015 mm, 0.02 mm, 0.025 mm, 0.03 mm, 0.05 mm, 0.08 mm, 0.1 mm, 0.15 mm, 0.2 mm, or 0.5 mm.

In some embodiments, the sample stage is coupled with one or more fluidic control devices e.g., 3830 in FIG. 38. The fluidic control device may be in fluidic communication with the sample stage (e.g., the flow cell devices) and may be configured to hold, dispense, and collect various fluids that are used in sequencing reactions in the flow cell devices in a sequencing run. The fluidic control device can be individually connected fluidically with the sample(s) on its corresponding top stage.

FIG. 38 shows 5 different fluidic control devices, each in fluidic connection with sample(s) of a corresponding top stage. In some embodiments, the fluidic control devices can be immobilized relative to the base stage. In some embodiments, the fluidic control devices can be immobilized relative to the corresponding top stage. In some embodiments, each fluidic control device can include a dispenser that is configured to dispense one or more reagents to the samples. For example, the dispenser may dispense openly the reagents to a corresponding inlet of a flow cell. As another example, the dispenser may be connected to the inlet of the flow cell devices via tubing, and the reagents can travel through the tubing to contact the sample(s) in the flow cell device. In some embodiments, the fluidic control device may include one or more pumps to facilitate dispensing of fluids to the samples and/or collection of fluids from the samples.

In some embodiments, flow cell devices on multiple top stages may share a single flow control device for simplicity of the system, lower system cost, and less waste of sequencing reagents in comparison to sequencing systems using multiple flow control devices. In such embodiments, different tubing may be used to enable fluidic communication to different flow cell devices on different top stages. Such different tubing may be for different reagents or identical reagents. Alternatively, different dispensing tips may be used to allow fluidic administration to the different flow cell devices on different top stages. In some embodiments, same dispensing tips may be shared among different top stages, and reagent dispensing can be done in a sequential manner over time.

In some embodiments, the sample(s) may be immobilized on a solid support, e.g., a flow cell, to be imaged using the optical system. The flow cell may include one or more lanes, each lane corresponding to a microfluidic channel that allows sequencing reagents or other fluids, e.g., washing buffers, in a sequencing run to flow therethrough. In some flow cells with two lanes, the lanes are positioned parallel to each other. In some embodiments, the sample stage herein may utilize flow cells with a lane orientation that is different from these flow cells. In some embodiments, the flow cells herein may include multiple lanes, and each pair of lanes may be positioned with an acute angle between their longitudinal directions so that they are not parallel to each other. For example, multiple lanes may be positioned axially along different radii of the sample stage, e.g., top stage with a predetermined angle between each adjacent pair of lanes. In such embodiments, the motion of the top stage along the y-axis of the top stage relative to the base stage can be eliminated. Instead, the base stage can be rotated at a predetermined angle to move to a next lane of the sample flow cell. Such embodiments with non-parallel lanes may advantageously remove the need for moving the top stage along its corresponding x-axis, thereby simplifying the motion in the x-y plane of the top stage relative to the base stage.

In some embodiments, the top stage here may include a manifold that can securely holds one or more flow cell devices here. The manifold may include an open state in which the flow cell device(s) can be removed or installed in the manifold. The manifold may also include a closed state in which the flow cell device is secured therewithin, and the sealed fluidic communication between the flow cell device (e.g., the cleaning outlet) and the manifold is formed. Further, in the closed state, the relative position of the flow cell device to the manifold is fixed. In some embodiments, the manifold includes a sealed fluidic communication with the fluidic control device.

In some embodiments, the fluidic control device includes one or more sealed fluidic pathways to the manifold and the flow cell devices. In some embodiments, some of the sealed fluidic pathways are configured for sealed fluidic administration to the flow cell devices. In some embodiments, some or all of the sealed fluidic pathways are configured for sealed fluidic collection from the flow cell devices (e.g., cleaning fluidic residuals from the inlet of the flow cell devices).

In some embodiments, the sample stage and optical systems and optical assemblies described herein advantageously remove the need for movement of the sample stage along the z-axis relative to the optical assembly or optical system herein. As such, the possible problems and complexity of moving the sample stage and the sample in z-direction are also eliminated. Z movement to achieve focusing of the sample(s) can be performed by moving individual lens element(s), e.g., a single lens element, of the imaging module relative to the housing thereof, which can be more simple, convenient, and accurate compared to some optical systems.

In some embodiments, disclosed herein are methods of sequencing multiple DNA samples positioned on a rotary sample stage for DNA sequencing using various sequencing methods including but not limited to sequencing by synthesis, sequencing by avidite, sequencing by binding. Such methods may be repeated in one or more sequencing cycles in a sequencing run.

In some embodiments, the methods of sequencing multiple DNA samples positioned on a rotary sample stage for DNA sequencing comprises an operation of obtaining a sample stage comprising the base stage and the one or more top stages positioned on the top surface of the base stage, wherein the base stage is rotatable about a z-axis relative to the optical system or the imaging module of a sequencing system.

In some embodiments, the methods comprises an operation of positioning and securing a first flow cell device relative to a first top stage of the one or more top stages.

The flow cell device can have 2D or 3D samples embolized thereon. The flow cell device can have various number of microfluidic channels with channel surfaces that the sample(s) can be immobilized on. The flow cell device herein may have 2, 3, 4, or more channel surfaces. The multiple channel surfaces may be displaced from each other along the z axis so that at least 2, 3, or more channel surfaces are at 2, 3, or more different z locations relative to the optical system. For example, the flow cell device may have 2 channels along the z direction so that it has 4 surfaces at different z locations.

The flow cell devices may be secured relative to the top stage so that there is no relative movement between the flow cell device and the corresponding top stage when the top stage moves. The flow cell device may be secured via various securing or fastening means including but not limited to mechanical clamping the flow cell device down, fastening with a magnetic or electro-magnetic force, positioning the flow cell device into a fitted housing (e.g., the manifold) which is fastened to the top stage, coupling a pin or post of the stage to a hole or a grove of the flow device. For example, the flow cell device can be secured in its corresponding manifold in a closed state, and sealed fluidic communication between the flow cell device and the manifold can be established in the closed state.

In some embodiments, the methods further comprise an operation of positioning and securing a second flow cell device relative to a second top stage of the one or more top stages. The second top stage can have identical or different securing or fastening means as the first top stage.

In some embodiment, the methods further comprise an operation of dispensing, by a first fluidic control device, one or more sequencing reagents to the first flow cell device positioned on a first top stage so that the samples can undergo sequencing reactions. Such operation of dispensing sequencing reagents can be performed openly, e.g., via a dispensing tip to an open area that leads to the channel(s) of the flow cell device. Alternatively, such operation of dispensing sequencing reagents can be performed via closed tubing.

In some embodiments, the method further comprises imaging a first sample region of the first flow cell device using the optical system of the sequencing system. The first sample region can include at least part of a first tile of the flow cell device. Such operation of imaging may include collect emitted optical signals from the sample(s) by an image sensor of the imaging module. Such operation may also include autofocusing the imaging module on the samples using various autofocusing methods. Such operation of imaging may also include generate excitation light that travels to the sample(s).

After completion of the imaging operation of the first sample region of the first flow cell device, the methods may include an operation of moving the first top stage within the x-y plane relative to the optical system while preventing the second flow cell device from moving relative to the optical system. Such operation may enable the second sample region, e.g., at least part of a second tile, to be positioned properly for imaging. Such movement can be along the x, y, or any other direction within the sample plane, e.g., the x-y plane. The base stage may remain still relative to the optical system during such movement of the first top stage.

In some embodiments, the methods further include an operation of moving the first fluidic control device so that the first fluidic device, e.g., its dispensing tip(s), stay still relative to the first flow cell device while the first flow cell device moves relative to the optical system. In some embodiments, the first fluidic control device does not need to be moved, e.g., when closed tubing connects the first fluidic control device to the first flow cell device while the first flow cell device moves relative to the optical system. In some embodiments, the first top stage is actuated by the first motor that is configured to actuate the base stage. In some embodiments, the first top stage is actuated by a second motor that is configured to actuate one or more top stages independent of actuation of the base stage.

In some embodiments, the operation of moving the first top stage within the x-y plane relative to the optical system while preventing the second flow cell device from moving relative to the optical system comprises: moving the first top stage along a radius of the top surface of the base stage with a predetermined distance relative to the optical system independently while preventing the second flow cell device from moving relative to the optical system.

In some embodiments, the operation of moving the first top stage within the x-y plane relative to the optical system while preventing the second flow cell device from moving relative to the optical system comprises: moving the first top stage along a direction orthogonal to a radius of the top surface of the base stage with a predetermined distance relative to the optical system independently while preventing the second flow cell device from moving relative to the optical system.

In some embodiments, the methods further include an operation of imaging the second sample region of the first flow cell device using the optical system of the sequencing system.

After completion of imaging the second sample region of the first flow cell device, the methods can further include an operation of moving the first top stage and imaging other sample regions of the first flow cell device till all the desired sample regions of the first flow cell device have been imaged.

In some embodiments, the methods may further include an operation of rotating the sample stage with a predetermined angular resolution to position the second flow cell device in a predetermined position relative to the optical system. This operation may occur in a same sequencing cycle with the operations of imaging the sample regions of the first flow cell device. Alternatively, this operation may occur in a different sequencing cycle with the operations of imaging the sample regions of the first flow cell device. The angular resolution can be the first resolution disclosed herein.

After rotating the sample stage with the predetermined angular resolution to position the second flow cell device in the predetermined position relative to the optical system, in some embodiments, the methods may further include an operation of dispensing by a first fluidic control device or a second fluidic control device, one or more sequencing reagents to the second flow cell device so that the samples immobilized on the second flow cell device can undergo sequencing reactions. The one or more sequencing reagents dispensed to the second flow cell device can be different from those dispensed to the first flow cell device, e.g., for different sequencing chemistry or applications. This operation of dispensing sequencing reagents to the second flow cell device may be optional, for example, if the same sequencing reagents are used and are dispensed simultaneously to the first flow cell device and the second flow cell device before imaging the first flow cell device.

In some embodiments, the method further comprises: moving the first fluidic control device or the second fluidic control device to position the second fluidic cell device in a predetermined position relative to the first fluidic control device or the second fluidic control device.

After rotating the sample stage with the predetermined angular resolution to position the second flow cell device in the predetermined position relative to the optical system, in some embodiments, the methods may further include an operation of imaging a first sample region of the second flow cell device using the optical system of the sequencing system, e.g., at least part of a first tile of the second flow cell device. The imaging operation of the first sample region of the second flow cell device can be identical to the imaging operation of the sample regions in the first flow cell device. The imaging operation can be different from the imaging operation of the first sample region of the second flow cell device, for example, when 2D samples are included on the first flow cell device while 3D sample(s) are included on the second flow cell device so that the imaging operation may including imaging different z levels for 3D sample(s) while imaging at a single z level for 2D sample(s).

In some embodiments, the rotary sample stage and methods of sequencing the samples using the rotary stage can advantageously improve sequencing capability and system throughput by allowing multiple flow cell devices to be sequenced and imaged using a single sample stage, and such multiple flow cell devices may contain different samples that can undergo different sequencing reactions. Further, in some embodiments, the rotary sample stage and methods of sequencing using the rotary stage can advantageously improve sequencing efficiency by allowing imaging of a first flow cell device to be performed simultaneously while dispensing and flowing sequencing reagents to a second flow cell device.

In some cases, an optical system can comprise a stage. The stage can be as described elsewhere herein (e.g., can be configured to hold a flow cell device, a slide, or similar otherwise solid support with samples thereon). The stage may lack movement in an optical axis of the system. Such movement may be relative to a non-movable housing of the optical assembly, e.g., the housing of the first or third element. For example, for a system configured to illuminate and image a sample at multiple different z levels along the z-axis of the sample, e.g., an in situ sample of cells or tissue, the stage may lack any movement along the z-axis relative to the non-movable housing of the optical system, e.g., the housing of the first or third element of the optical assembly during imaging of the sample. In another example, the stage and the optical assembly may not move along a z axis relative to each other but can still enable focusing of the sample so that it is within the focal plane of the optical assembly in the z-axis to focus prior to or during imaging. The optical system herein may utilize a method of focusing by moving only one or more lens element of the optical assembly instead, such as moving the stage or the optical assembly in order to move the sample into a focal plane of the optical system. The one or more lens element of the optical assembly may not include an objective lens. In some embodiments, the optical system herein may utilize a method of focusing disclosed herein that eliminates the z-stage that is response for moving the sample along z axis relative to the objective lens in another optical systems.

The solid support (e.g., flow cell) can comprise a probe configured to bind a nucleic acid molecule, a protein, a polypeptide, or the like. For example, a probe complementary to a nucleic acid molecule can be immobilized (e.g., bound) on a surface of the solid support.

The solid support (e.g., flow cell) may comprise one or more samples immobilized thereon. For example, the solid support can comprise one or more probe molecules configured to bind one or more samples thereto. The solid support can comprise two or more surfaces with one or more samples immobilized thereto. For example, the solid support can comprise a first surface with a first sample immobilized thereto and a second surface with a second sample immobilized thereto. In some cases, the solid support comprises at least about 2, 3, 4, 5, 6, or more surfaces. In some cases, each surface of the solid support has a different sample immobilized thereto, and each sample can be illuminated and imaged by the optical system. In some cases, the two or more surfaces of the solid support can be axially displaced from each other along an optical axis (e.g., a z-axis) of the optical system. For example, the surfaces of the solid support can be in a stacked configuration with regard to the optical axis of the optical system.

The optical system can comprise a light source configured to illuminate the flow cell. The illumination can be used in at least a portion of an imaging operation (e.g., a sequencing operation as described elsewhere herein). The illumination may have an irradiance of at least about 10, 15, 20, 25, 30, 35, 40, 45, 50, 55, 60, 65, 70, 80, 90, 100, or more milliwatts per square millimeter. In some cases, the light source may be a pulsed light source (e.g., a flash lamp, a pulsed laser, a pulsed light emitting diode, etc.). In some cases, the light source can be a continuous light source (e.g., an incandescent source, a fluorescent lamp, a light emitting diode, a continuous laser, etc.).

The optical system can comprise an optical assembly. The optical assembly can be disposed within an optical path between from the stage and to the light source. The optical assembly can be configured to provide an illumination of the flow cell of greater than about 5, 6, 7, 8, 9, 10, 11, 12, 13, 14, 15, 16, 17, 18, 19, 20, 25, 30, 35, 40, 45, 50, 55, 60, 65, 70, or more square millimeters. The optical assembly can be configured to illuminate the area of the flow cell with a variation (e.g., a peak-to-valley variation, standard deviation, variance, interquartile range, mean absolute deviation, coefficient of variation, etc.) of at most about 25, 24, 23, 22, 21, 20, 19, 18, 17, 16, 15, 14, 13, 12, 11, 10, 9, 8, 7, 6, 5, 4, 3, 2, 1, or less percent. The optical assembly can be configured to illuminate the area of the flow cell with a variation (e.g., a peak-to-valley variation, standard deviation, variance, interquartile range, mean absolute deviation, coefficient of variation, etc.) of at most 10, 9, 8, 7, 6, 5, 4, 3, 2, 1, or less percent. In some embodiments, such illumination variation can be the variation in power cross the area of the flow cell. In some embodiments, such illumination variation can be the variation in irradiance cross the area of the flow cell.

In some cases, the optical assembly does not comprise an objective lens (e.g., objective lens assembly). For example, the optical assembly may not comprise an objective lens assembly. In some cases, the optical system does not comprise an objective lens. For example, the entire optical system may not comprise an objective lens anywhere in the optical system. In another example, the optical system may not comprise an objective lens in the optical path of the optical system. The optical assembly may not comprise a tube lens. In some cases, the optical system may not comprise a tube lens. For example, there may not be a tube lens in the optical path of the optical system. Despite not having a tube lens and/or an objective lens, the optical assembly or optical system may be able to achieve the large area illumination described herein. For example, the optical system can achieve broad, flat illumination without the use of a tube lens or objective lens.

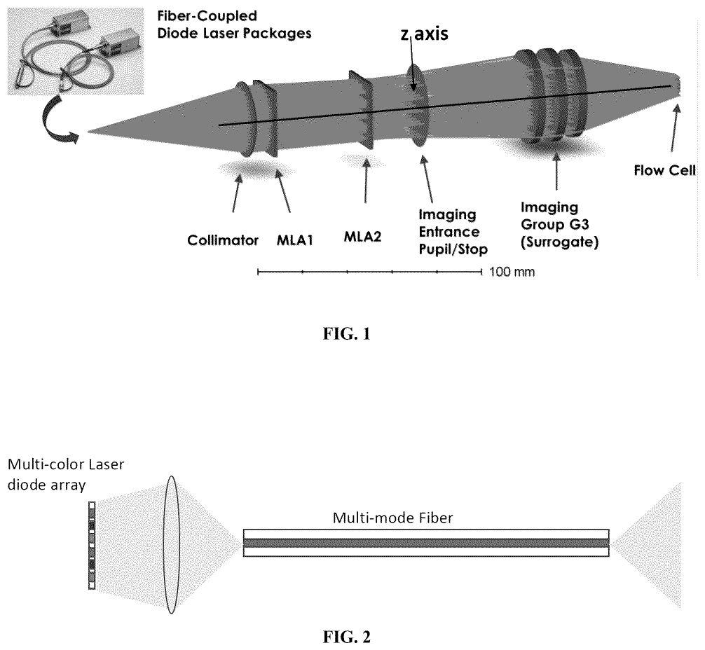

The optical assembly, more specifically, the illumination system of the optical assembly (e.g., as shown in FIG. 1) can be configured to transmit the illumination light from the light source to the stage and a sample immobilized on the stage. For example, the illumination system may be positioned above the excitation dichroic filter, e.g., above 2770 as shown in FIG. 27, so that the excitation dichroic filter of the optical assembly may be configured to transmit the illumination from the illumination system to the sample and the stage, via the first segment. In other words, the excitation dichroic filter and the optical elements of the optical assembly that are between the excitation dichroic filter and the stage are within the optical path between the light source and the stage, e.g., first segment 2710 and 2770 in FIG. 27. In some embodiments, the excitation dichroic filter can also be configured to reflect emission light from the sample to an optical path of a detector. In other words, the excitation dichroic filter and the optical elements of the optical assembly that are also between the excitation dichroic filter and the stage are within the optical path between the stage and the detector, e.g., first segment 2710 and 2770 in FIG. 27. The optical assembly can be configured to receive an emission light from the flow cell (e.g., a light produced by the interaction of the illumination light with a label in a sample in the flow cell). For example, the optical assembly can be configured to receive the emission light and transmit the emission light to a detector. Nonlimiting examples of the detection includes a CCD camera or a CMOS camera. The optical assembly may have a numerical aperture of at least about 0.2, 0.3, 0.4, 0.5, 0.6, 0.7, or more. The emission light may have a wavelength of at least about 400, 450, 500, 550, 600, 650, 700, 750, 800, 850, 900, 950, 1,000, or more nanometers. The emission light may have a wavelength of at most about 1,000, 950, 900, 850, 800, 750, 700, 650, 600, 550, 500, 450, 400, or less nanometers. The emission light may have a wavelength in a range as defined by any two of the preceding values. The optical assembly can have a working distance of at least about 1 mm to about 30 mm. The optical assembly can have a working distance of at least about 2 mm to about 25 mm.

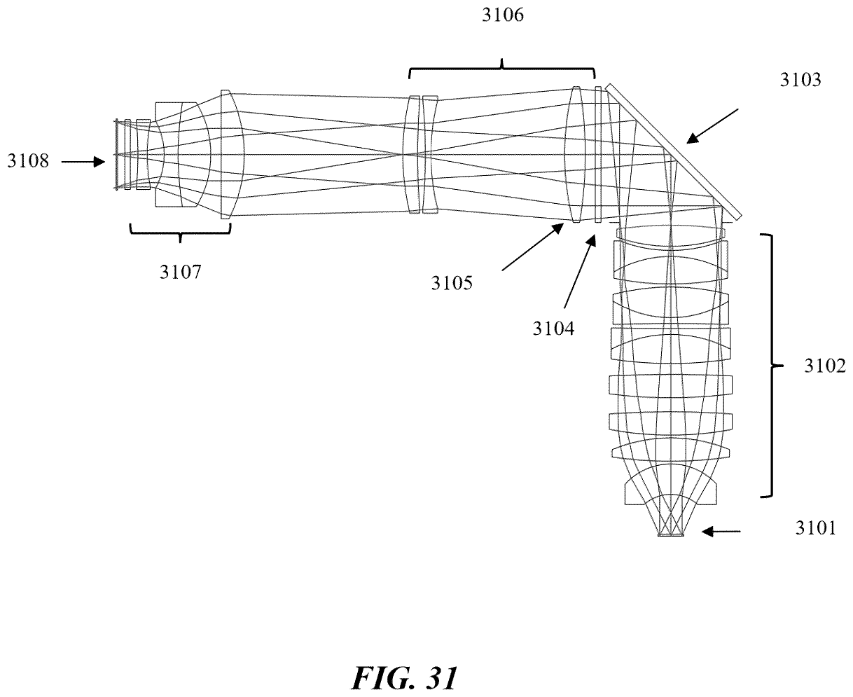

In some cases, the optical assembly can comprise a motion coil housed within the optical assembly. The motion coil can be configured to move a focusing element within an optical path of the optical system. For example, the motion coil can be used to move a focusing element to change the focus of the optical system without moving other portions of the system (e.g., the optical assembly, the stage, etc.). Alternatives to a motion coil include, but are not limited to, piezoelectric actuators, motors (e.g., stepper motors, servo motors, etc.), electrostatic actuators, hydraulic actuators, pneumatic actuators or the like, or any combination thereof. In some cases, the motion coil (or other actuator) can be positioned external to the optical assembly or optical system and be configured to move the focusing element along the optical axis. For example, a motor positioned outside of the optical assembly can be operatively coupled to the focusing element and can adjust the position of the focusing element within the optical assembly. 2760 of FIG. 27 shows an actuator integrated with an optical assembly. An example of an external actuator can be found in FIGS. 29A-29B, where actuator 2901 is coupled to the focusing element 2903 by coupling element 2902. In some cases, the actuator is coupled directly to the focusing element or a focusing element housing. For example, the actuator can be coupled without the use of a coupling element. In some embodiments, the focusing element includes one or more lenses of the optical assembly. In the particular embodiments as shown in FIG. 29A and FIG. 31, the focusing element is a single lens of the optical assembly. The focusing element can be mechanically coupled with a focusing element housing so that movement of the focusing element housing may cause movement of the focusing element. Various methods may be used for the mechanical coupling between the focusing element and the focusing element housing. Such mechanical coupling can be direct without contact of a third element. Such mechanical coupling can be indirect with contact of a third element in between. For example, as shown in FIG. 29A, the two ends of the focusing element 2903 are clamped directly with the focusing housing element 2904.

The optical system may have a composite root mean square (RMS) error of the wavefront of light transmitted through the optical system of at most about 0.2, 0.15, 0.14, 0.13, 0.12, 0.11, 0.1, 0.9, 0.8, 0.7, 0.6, 0.5, 0.4, 0.3, 0.2, 0.1, or less. The optical assembly or the optical system may have an illumination efficiency (e.g., an efficiency of the illumination light transmitted through the optical assembly or optical system) of at least about 80, 85, 90, 91, 92, 93, 94, 95, 96, 97, 98, 99, 99.9, or more percent. The optical system may be configured to image the solid support without moving an optical compensator into, out from, or both into and out from the optical path between the solid support and detector of the optical system. For example, an image of a sample can be taken without moving an optical compensator into or out of the optical path between the sample and the detector. The optical assembly may be configured to generate one or more spatial constrictions lateral to the optical paths of light traveling through the optical assembly (e.g., one or more double waists in the light). The one or more spatial constrictions can advantageously: enhance imaging resolution; enhance and the depth of focus of the optical assembly, provide the low illumination variation of the optical assembly at the sample stage, provide wide field of view with low illumination variation, or a combination thereof. The optical assembly may be configured to generate at least one field curvature corrections lateral to the optical path of light traveling through the optical assembly. At least one field curvature correction can similarly improve optical resolution and depth of focus. The optical assembly may be configured to generate more than two field curvature corrections lateral to the optical path of light traveling through the optical assembly.

FIG. 27 shows an example of an optical assembly, according to some embodiments. The first segment 2710 can comprise a first housing including a first plurality of lenses therewithin. The second segment 2720 can comprise a second housing. The third segment 2730 can comprise a third housing containing a second plurality of lenses therewithin. The first segment and the third segment can be optically aligned (e.g., aligned in a same optical axis). The first segment can be positioned between the third segment and the stage (e.g., solid support 2740). The third segment can be positioned between the first segment and an image sensor (e.g., detector) of the optical system 2750. One or more lens element of the first plurality of lenses can be movable along an optical path of the optical system by at least about 0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or more millimeters. The first plurality of lenses can be movable along an optical path of the optical system by at most about 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, or 0 millimeters. One or more lens elements of the first plurality of lenses can be movable along an optical path of the optical system over a distance in a range as defined by any two of the preceding values. For example, a single lens element of the first plurality of lenses can be movable along an optical path of the optical system in a range of about 0 to about 25 millimeters in one direction or both directions along the optical axis. The optical assembly may comprise a dichroic 2770 configured to transmit an illumination light from outside the optical assembly to the solid support.

In some cases, the first plurality of lenses comprises one or more asymmetric convex-convex lenses. In some cases, the second plurality of lenses comprises one or more asymmetric concave-concave lenses. The use of both the convex-convex and concave-concave lenses and the adjustment between the lenses can be used to tune the focal plane of the optical system (e.g., move the focal plane to image various surfaces of the solid support).

In some cases, an actuator (e.g., a motion coil) can be coupled to a portion of the optical assembly (e.g., the first, second, or third housings of the optical assembly), and the focusing element can be coupled to the portion of the optical assembly the actuator is coupled to. In this way, the actuator can adjust a focus of the optical assembly. The optical assembly can be configured to generate at least one field curvature correction lateral to an optical path of the first, second, or third segments.

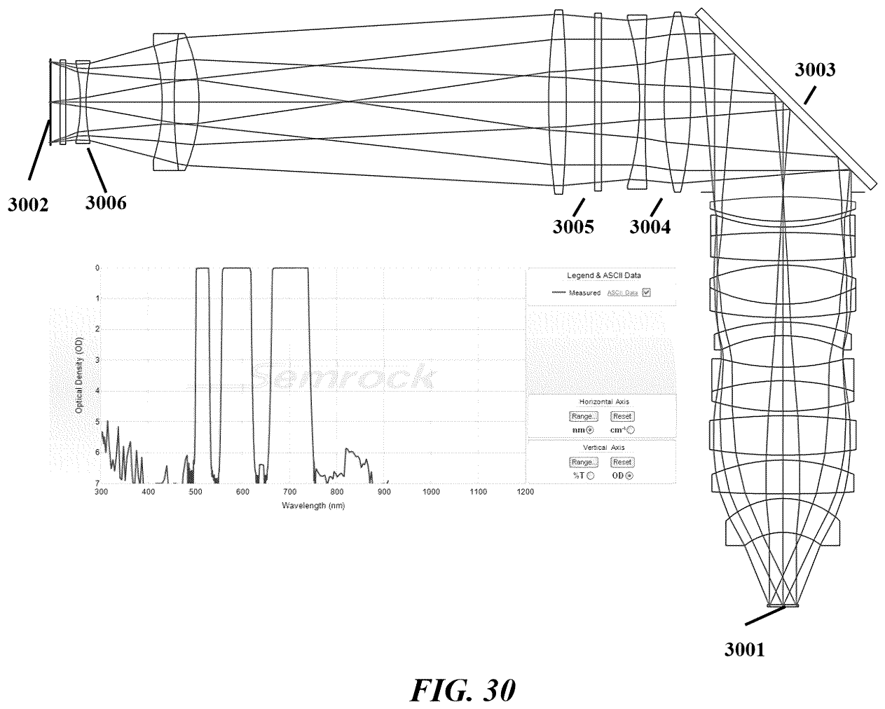

FIG. 30 shows an example of the optical elements of an optical assembly and the associated focus paths, according to some embodiments. The optical assembly can be configured to focus light onto and between a solid substrate 3001 and a detector 3002. For example, illumination light can come through dichroic filter 3003 and be focused on the solid substrate 3001. Once the illumination light interacts with a sample on the solid support, the resultant sample light can be refocused up the optical assembly, reflected off of the dichroic filter, and transmitted through internal focus group 3004. The internal focus group may be configured to adjust a focus of the optical assembly, thereby permitting imaging of a plurality of surfaces of the solid support. The internal focus group may be coupled to an actuator as described elsewhere herein. The signal light can be transmitted through multi-bandpass filter 3005. The multi-bandpass filter may have an optical density plot as shown in the inset graph of FIG. 30. The multi-bandpass filter may be configured to transmit sample light from a label in a sample while rejecting other light to reduce noise. Passing through a field flattener 3006, the signal light can be detected by detector 3002 and further analyzed as described elsewhere herein.

Similarly, FIG. 31 shows an alternate lens configuration for illuminating and collecting light from a solid support (e.g., flow cell, slide, etc.) 3101. Lens group 3102 can be configured to both focus incoming illumination light (e.g., illumination light transmitted through dichroic 3103) onto the solid substrate while also focusing signal light from the solid substrate back up through notch filter 3104 and through focusing element 3105 (part of lens group 3106). The focusing element can be as described elsewhere herein. Lens group 3017 can be configured to focus the signal light onto detector 3108 for processing as described elsewhere herein.

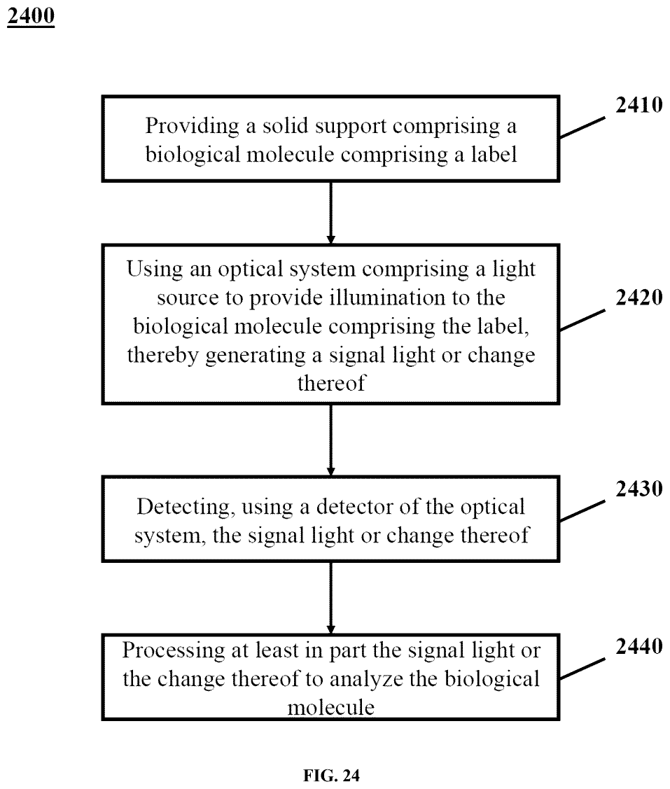

FIG. 24 shows a flow chart of a method 2400 of analyzing a biological molecule, according to some embodiments. In an operation 2410, the method 2400 may comprise providing a solid support comprising the biological molecule. The solid support may be a flow cell. The biological molecule may comprise a label. The biological molecule may comprise, for example, nucleic acid molecules, proteins, polypeptides, carbohydrates, lipids, or the like. The label may be changed depending on the identity of the biological molecule. For example, the label for a nucleic acid and a protein may be different to enable binding to the different molecules. In another example, for a nucleic acid, the label can be hybridized to the nucleic acid.

The label may be an optical label (e.g., a fluorescent label, a luminescent label, a Raman label, scattering label, plasmonic label, etc.), a magnetic label, or the like. In some cases, the probe is integral to the biological molecule. For example, a protein comprising green fluorescent protein can be the biological molecule and the label. In some cases, prior to operation 2410, the method 2400 may comprise binding the biological molecule to a probe in the solid support and/or coupling the label to the biological molecule. For example, the biological molecule can be flowed into a flow cell already comprising a probe, bind to the probe, and then a fluorescent label can be coupled to the biological molecule once it is bound to the probe. In another example, a biological molecule already comprising the probe can be flowed into the flow cell and bound to the probe. As another example, the sample to be sequenced and imaged may be immobilized on the flow cell device and the label or probe may be flowed into the flow cell during various sequencing reactions. Details of examples of sequencing the sample (s) are disclosed below.

In another operation 2420, the method 2400 may comprise using an optical system comprising a light source to provide illumination to the biological molecule comprising the label, thereby generating a signal light or a change thereof. The illumination may be provided over an area of the flow cell that is greater than about 5, 6, 7, 8, 9, 10, 11, 12, 13, 14, 15, 16, 17, 18, 19, 20, 25, 30, 35, 40, 45, 50, 55, 60, 65, 70, or more square millimeters. The illumination may have a variation (e.g., a peak-to-valley variation, standard deviation, variance, interquartile range, mean absolute deviation, coefficient of variation, etc.) of at most about 25, 24, 23, 22, 21, 20, 19, 18, 17, 16, 15, 14, 13, 12, 11, 10, 9, 8, 7, 6, 5, 4, 3, 2, 1, or less percent.

The optical system may be as described elsewhere herein. For example, the optical system may not comprise an objective lens or tube lens. The solid support may not be moved along an optical axis of the optical system. For example, a flow cell may be stationary along the z axis of the flow cell (e.g., the optical axis of the optical system) while being movable in the x and y axes of the flow cell. In some cases, a plurality of images of the flow cell can be acquired without moving the flow cell along the optical axis. For example, a first image at a first focal depth can be acquired and a second image at a second focal depth can be acquired without moving the solid support.

A motion coil or other actuator as described elsewhere herein may be used within the optical system to move a focusing element within an optical path of the optical system, thereby changing a focus of the optical system on the solid support. For example, an actuator can move the focusing element along the optical path to change a parameter of the optical system, thereby changing the focus of the optical system from one side of a flow cell to another. The light source may be a light source as described elsewhere herein (e.g., a pulsed light source). The optical system may have an illumination efficiency (e.g., an efficiency of the illumination light transmitted through the optical system) of at least about 80, 85, 90, 91, 92, 93, 94, 95, 96, 97, 98, 99, 99.9, or more percent.

In another operation 2430, the method 2400 may comprise detecting, using a detector of the optical system, the signal light or the change thereof. The signal light may have a wavelength of at least about 400, 450, 500, 550, 600, 650, 700, 750, 800, 850, 900, 950, 1,000, or more nanometers. The signal light may have a wavelength of at most about 1,000, 950, 900, 850, 800, 750, 700, 650, 600, 550, 500, 450, 400, or less nanometers. The signal light may have a wavelength in a range as defined by any two of the preceding values. The optical system may have a numerical aperture of at least about 0.2, 0.3, 0.4, 0.5, 0.6, 0.7, or more. In some cases, the label can be removed from the biological molecule. For example, a hybridized label can be dehybridized from a nucleic acid molecule.

In another operation 2440, the method 2400 may comprise processing at least in part the signal light or the change thereof to analyze the biological molecule. The processing may be as described elsewhere herein (e.g., using an identity of the label to determine a portion of the biological molecule, etc.). An image of the solid support may comprise a field of view of greater than about 20, 25, 30, 35, 40, 45, 50, 55, 60, 65, 70, or more square millimeters. The processing may comprise processing at least in part the signal light or the change thereof to generate one or more images of the solid support and analyze the one or more images of the solid support to generate base calls of the sample.

In some cases, the method 2400 may comprise repeating operations 2420-2440 for an additional biological molecule coupled to an additional surface of the solid support. For example, a plurality of biological molecules can be coupled to the solid support with a plurality of labels each affixed to the plurality of biological molecules, and the optical system can image each of the labels. In some cases, the method 2400 may comprise repeating operations 2410-2440 for an additional label that binds to another portion of the biological molecule. For example, a first label can identify a first nucleotide of a nucleic acid, the first nucleotide can be removed from the nucleic acid, and a second label can be hybridized to the nucleic acid molecule at a second nucleotide. In this example, the second label can be identified in a similar way to the first, providing information related to the second nucleotide of the nucleic acid molecule.

### Illumination Systems

The illumination system herein may advantageously provide an extra-wide illumination field that is no less than 10 mm², 20 mm², 30 mm², 40 mm², or 50 mm² at a sample plane. In some embodiments, the illumination system may advantageously provide an illumination field that is 2×, 4×, 5×, 6×, 8×, 10×, or 15× larger than the illumination fields generated by other illumination systems in NGS optical systems. The illumination system may advantageously provide an illumination power density that is no less than 30, 40, 50, 60, or 70 milli-Watts/mm² at the sample plane. In some embodiments, the illumination system is configured to generate an illumination field at the sample stage that is greater than 20, 30, 40 or 50 mm² with less than +2%, 5%, 8%, 10%, or 12% variance or standard deviation in illumination power density across the illumination field. In some embodiments, the variance or standard deviation is measured as a percentage to an average power density, a maximum power density, or a median power density.

The illumination system advantageously enables imaging of wide FOVs that are 2×, 4×, 5×, 6×, 8×, 10×, or 15× larger than the largest FOVs provides by existing optical systems for NGS applications, thereby increasing system throughput and flexibility in NGS applications. With the capability of illuminating a wide field and imaging across some or all of the wide illumination field, the illumination system and imaging module herein can advantageously eliminate the problem of photon bleaching in un-imaged areas associated with some optical systems.

In some embodiments, the illumination system may provide a power efficiency of no less than 65%, 70%, 75%, or 80%. In other words, the power loss within the illumination system can be less than 20%, 25%, 30%, or 35%. In some embodiments, the illumination subsystem, the light beam delivery subsystem, or both may each provide a power efficiency of no less than 65%, 70%, 75%, 80%, 85%, or 90%. In some embodiments, one or more optical elements in the illumination system may each provide a power efficiency of no less than 65%, 70%, 75%, 80%, 85%, or 90%, e.g., power efficiency of the optical fiber(s), the lens arrays, etc. In some embodiments, the power efficiency can be determined as the ratio or percentage of the power exiting the optical element to the power entering the optical element.

The sample plane herein can be where the sample is positioned and can be orthogonal to a z-axis or optical axis of the imaging module. In some embodiments, the sample plane overlaps with a focal plane of the objective lens of the imaging module.

In some embodiments, the illumination system includes an illumination subsystem and a light beam delivery subsystem optically coupled to the illumination system.

### Illumination Subsystems

The illumination subsystem may include a light source, alone or in combination with a despeckler of the light source.

The light source can comprise one or more lasers. The lasers can be of various types. Some non-limiting examples of the lasers include: gas lasers, solid-state lasers, fiber lasers, dye lasers, and semiconductor lasers (laser diodes). The one or more lasers may comprise one or more laser diodes.

The one or more lasers may emit light of multiple wavelengths. In some embodiments, each laser or laser diode may emit light of a predetermined color, e.g., red, green, or blue. In some embodiments, each laser or laser diode may emit light within a wavelength range that corresponds to a predetermined color, e.g., red, green, or blue. The wavelength range of the predetermined color may be less than 0.1 Hz, 1 Hz, 10 Hz, 20 Hz, 50 Hz, or more. In some embodiments, the one or more lasers or laser diodes may emit light of multiple colors or wavelength ranges. In some embodiments, each laser or laser diode may emit light of multiple colors, e.g., a white light.

In some embodiments, the light source comprises one or more multi-color laser arrays. Each multi-color laser array can include lasers arranged in an array in any direction(s) in the x-y plane, e.g., a 2D array. The lasers in the array can be of different colors so that each laser can have a different color with its immediately adjacent neighbor(s) in the array. In some embodiments, the multi-color laser array comprises an array of lasers that emits laser light at 2, 3, 4, 5, or 6 wavelengths or in 2, 3, 4, 5, or 6 wavelength ranges. Each wavelength or wavelength range can correspond to a different color. In some embodiments, the multi-color laser array comprises lasers that emit light of 2, 3, or 4 color wavelengths or wavelength ranges at least in a direction that is orthogonal to a z axis. FIG. 2 shows an example of an embodiment of the multi-color laser array with laser diodes generating at least three different colors, e.g., blue, red, and green.

In some embodiments, the illumination subsystem may further comprise one or more optical fibers that can be coupled to the light source for transmitting light therefrom. In some embodiments, a single fiber is coupled to a corresponding laser or laser diode, either multi-colored or single colored. The optical fibers may be of various fiber lengths. For example, one or more of the optical fibers may be 0.5 m to 5 m long. In some embodiments, one or more optical fibers may include a fiber core. The fiber core may have a maximum dimension (e.g., diameter) that is 50 μm to 2000 μm in its cross section. The cross-section can be orthogonal to the longitudinal axis extending along the length of the fiber. In some embodiments, the cross section of the fiber core can be circular or substantially circular. FIGS. 1, 3-4, and 7 show examples of embodiments of the laser diode(s) and the optical fiber(s) coupled to the laser diode(s).

In some embodiments, the illumination subsystem further comprises a single optical fiber as shown in FIG. 2. The single optical fiber can be a multi-mode fiber. The multi-mode fiber is configured to transmit lights of different colors, different wavelengths, or different wavelength ranges in the same fiber. The single fiber may include a fiber core with a maximum dimension (e.g., diameter) of 400 μm to 2000 μm in its cross section orthogonal to the longitudinal axis of the fiber. The single fiber may include a fiber core with a maximum dimension (e.g., diameter) of 600 μm to 1600 μm. The single fiber may include a fiber core with a maximum dimension (e.g., diameter) of 800 μm to 1300 um.

In some embodiments, the illumination subsystem may comprise a plurality of optical fibers. Each optical fiber may be optically coupled to one or more corresponding lasers of the light source. The one or more corresponding lasers may emit light of a same wavelength or wavelength range. The one or more corresponding lasers may emit light of a same color. In some embodiments, the illumination subsystem further comprises one or more dichroic filters, optical lens elements, or both.

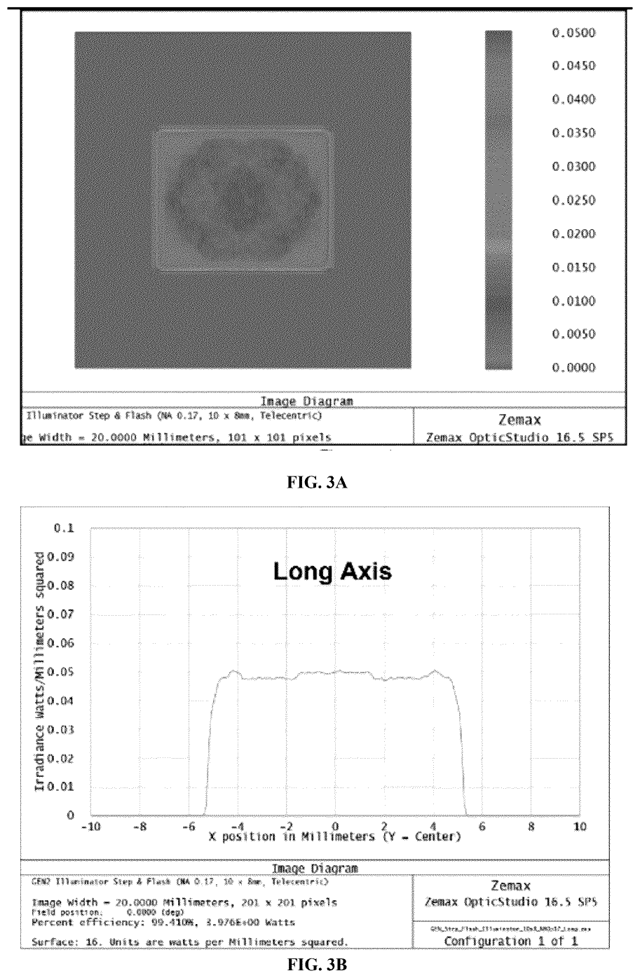

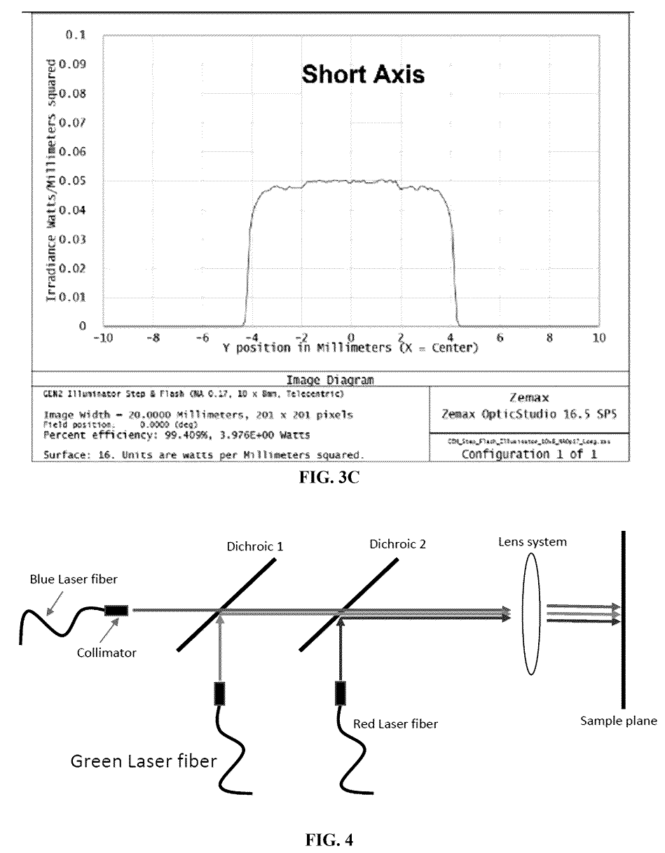

FIG. 3 shows an example of an embodiment in which the light source comprises a laser diode array of red, green, and blue colors. An individual fiber is coupled to a laser, and light with different colors and from different fibers can be combined together using various optical elements such as dichroic filters and lens elements.

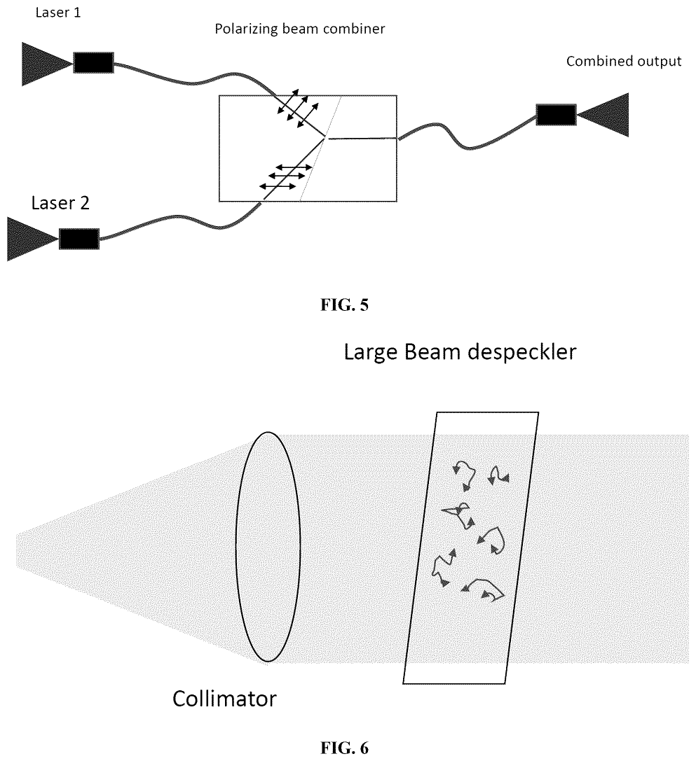

In some embodiments, the light source comprises one or more light beam combiners. Each light beam combiner is configured to combine two different light beams (e.g., different polarization or other beam characteristics) into a combined light beam. In some embodiments, the light beam combiner can be polarization light beam combiners. In some embodiments, the light source may include two or more lasers that emit light at a same wavelength or in a same wavelength range. Each light beam combiner may combine light emitted from such two or more lasers at the same light wavelength or in the same light wavelength range into a combined beam. In some embodiments, each light beam combiner may combine light emitted from two or more lasers emitting light of a same color into a combined light beam. In some embodiments, the light beam combiner is configured to increase power coupling into a fiber or at sample plane by combining two light beams into a combined light beam. In some embodiments, the power of the combined light beam is greater than each individual light beam before the combination. FIG. 5 shows an example of an embodiment combining two light beams of the same color from two lasers into a combined light beam with a greater power than each individual beam.

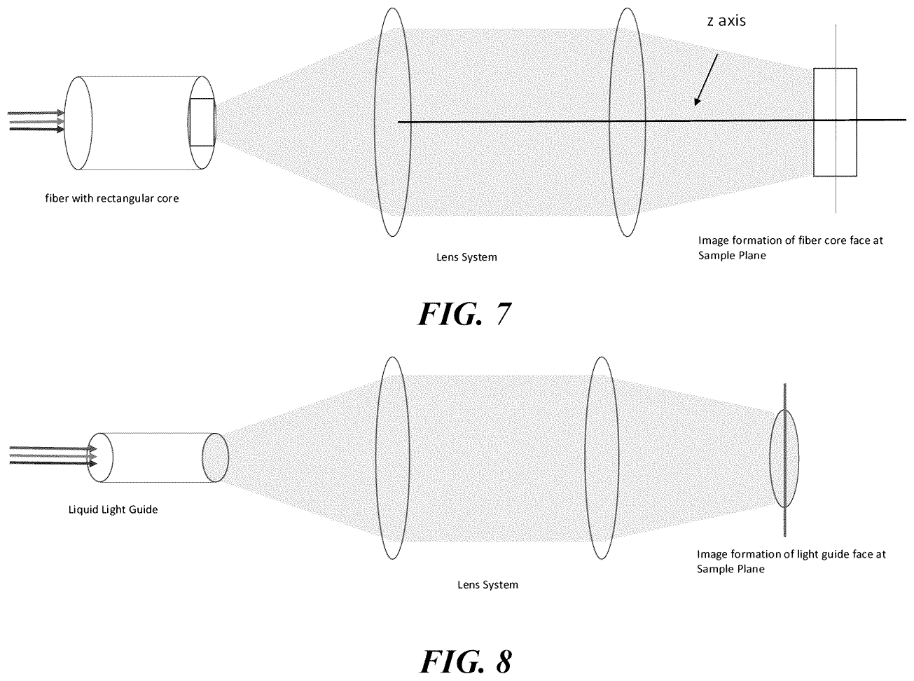

In some embodiments, the optical fiber, either coupled to a single color or multiple color laser, may comprise a core with a non-circular cross-section that is orthogonal to the z-axis. The non-circular cross-section may be of various shapes, e.g., an oval, a triangle, a diamond, a pentagon, a hexagon shape, etc. For example, the fiber core may include a rectangular or square cross-section. FIG. 7 shows an example of an embodiment of the optical fiber with a rectangular core which may advantageously facilitate delivery of more uniform light power at the sample plane in a rectangular shape that better matches the imaging FOV, which is also rectangular.

In some embodiments, the light source may be coupled to a liquid light guide with a liquid core. In some embodiments, the one or more liquid light guides are optically coupled to the light source in the absence of an optical fiber. In some embodiments, each laser may be coupled to a liquid light guide. In other embodiments, multiple individual lasers can be coupled into a single liquid light guide. FIG. 8 shows an example of an embodiment of the liquid light guide. In some embodiments, the liquid light guide can facilitate delivery of high optical power with homogenization across the wide illumination field at the sample plane. In some embodiments, the one or more liquid light guides comprise a liquid core. The liquid core may have a maximum dimension of 0.5 mm to 10 mm in the cross-section orthogonal to the z-axis. In some embodiments, the one or more liquid light guides comprise a liquid core of a maximum dimension of 0.2 mm to 20 mm in the cross-section orthogonal to the z-axis. In some embodiments, the liquid core comprises a cross-section that is circular. In some embodiments, the liquid core comprises a cross-section that is non-circular. In some embodiments, the liquid core may comprise a cross-section of various non-circular shapes.

In some embodiments, the illumination subsystem further comprises one or more coupling elements, e.g., optical lenses. The one or more coupling lenses can be positioned between the light source and an optical fiber, e.g., as shown in FIG. 2. The coupling lens(es) may be configured to couple the laser light from the light source to the optical fiber, the liquid light guide, or other optical element(s) for transmitting light, e.g., a collimator. The coupling lens(es) may include one or more of: an asymmetric convex-convex lens, a convex-plano lens, a concave-plano lens, an asymmetric concave-concave lens, and an asymmetric convex-concave lens.

FIGS. 26-27 shows a non-limiting example of the single channel time-sequential color imaging module or optical assemblies disclosed herein. The single channel time-sequential color imaging module herein may be advantageously used for imaging optical signal at different wavelengths, so that it is configured to performing imaging of multiple color channels in traditional systems without requiring additional image sensors and other optical elements such as the associated dichroic beam splitters and excitation notch filters. Compared to systems wherein signals are acquired by different channels, images of optical signals at different wavelengths, can be acquired in a sequential fashion in a single channel. The single channel time-sequential color imaging module advantageously reduces the system volume, complexity, and cost over existing optical systems.

FIG. 26 is a perspective view of the imaging module, showing the housing of different segments (e.g., 3 segments) of the imaging module. FIG. 27 is a cross-sectional view showing different lens elements of the different segments and their relative positions to each other. The autofocus light beam and excitation/illumination beam can be injected into the single channel time-sequential color imaging module through the excitation dichroic in FIGS. 26-27. The imaging module can have a “double-waist” design that includes at least two constrictions of the optical paths traveling through the imaging module, e.g., between the sample and the image sensor. For example, the two constrictions are shown in FIG. 31, one of the constrictions occurs in the first segment, and the other one may occur in the third segment. Compared to some imaging systems, these constrictions may induce over-corrected or a backward curving component to the field curvature correction, thereby enabling a flat-field imaging of the sample over a much wider dimension than these imaging systems may allow. This wider field may advantageously translate into greatly improved image acquisition times to cover a specific sample area, thereby offering the ability to build a high-throughput sequencing system. In this particular embodiment, the sample area or field-of-view that can be imaged within a single image is increased by a factor of 10×, 13×, 15×, or 20× over some imaging systems.

In some embodiments, a triple notch filter or a double notch filter (not shown) can be embedded in the collimated space between two lens elements, e.g., 5 and 7 in FIG. 28. The maximum angle of optical paths (with the optical axis) at this location is controlled to be less than 10, 8, 6, or 5 degrees to enable OD6 rejection of the excitation wavelengths. In some embodiments, a double-notch or triple-notch filter may be added in this location to suppress possible leakage from the excitation wavelengths, e.g., from the illumination system to the sample. For example, the triple or double notch filter may sit on top of 9 in FIG. 38, and the housing may provide an accessible hard aperture stop. The accessible hard aperture stop may be accessible from outside the imaging module.

In some embodiments, the imaging module here enables autofocusing at least along the optical axis by moving one or more elements along the optical axis. In some embodiments, a lens element may move longitudinally to enable multiple surface imaging with a z-motion in a range from 0 to 5 mm, 0 to 3 mm, 0 to 2 mm, 0 to 1 mm, 0 to 0.8 mm, 0 to 0.6 mm, 0 to 0.5 mm, or 0 to 0.4 mm. The movement of an internal lens element can advantageously eliminate the z-stage assembly for moving the entire objective lens relative to the sample and the associated problems with integration.

In some embodiments, the imaging module may include a lens element for aberration correction. The lens element may be aspheric. For example, 8 in FIG. 28, is a lens element for aberration correction. In some embodiments, this lens element may enable spherical aberration correction to help simultaneously improve aberration correction and improve transmission. In some embodiments, this lens element, or any other optical element, can be manufactured using glass types with low autofluorescence. In some embodiments, this lens element may include an all-spherical design.

In some embodiments, the imaging module or the optical assembly herein includes an illumination system. The illumination system may include the illumination subsystem and the light beam delivery system. FIG. 1 shows a nonlimiting example of the illumination system with excitation/illumination light.

### Despecklers

Due to the coherent feature of laser light, the interference of light waves with the same frequency may happen and cause undesired laser speckle noise. The speckle noise may cause inhomogeneity in the illumination field, thus error in DNA sequencing results. As such, there is a need for effective and easy-to-implement speckle reduction. Commercially available despecklers are expensive, e.g., $1000 per unit. The despecklers herein may provide a low-cost, easy to implement, and effective way of reducing the speckle noise of the light source.

In some embodiments, various laser-based illumination sources may suffer from speckle noise, and that speckle intensity may be controlled to minimize possible errors in sequencing analysis based on image intensities. The source coherence (e.g., speckle) may be reduced by using predetermined fiber length and/or fiber core size, e.g., a 1-3 m length of fiber with a 200-800 um core. In some embodiments, the illumination system may allow mode mixing to reduce speckle to prespecified levels. In some embodiment, a time-variant diffuser reduction method may be integrated into the beam path to improve the source coherency.

In some embodiments, a single optical fiber may be coupled to a corresponding laser or laser diode. In some embodiments, the characteristics of the optical fiber may be predetermined in order to reduce speckle of the laser. Some non-limiting characteristics of the optical fiber may include: fiber length, fiber bending radius, fiber core shape, fiber core size, fiber attachment to a vibration source, etc.

In some embodiments, the despeckler is comprised of an optical fiber, which is optically coupled to the light source. In some embodiments, the despeckler is coupled to or associated with the optical fiber to mitigate speckle of the light source. In some embodiments, the despeckler is coupled to or associated with the optical fiber so that the speckle reduction can be generated at some or all regions of the optical fiber.

In some embodiments, the despeckler comprises a mechanical vibration source, e.g., a vibration motor. The vibration source may produce vibration at a predetermined frequency or frequency range. In some embodiments, the mechanical vibration source is configured to vibrate at one or more frequencies in an audible sound frequency range, an ultrasound frequency range, or both. In some embodiments, the mechanical vibration source is configured to vibrate at one or more frequencies from 10 to 500 Hz. For example, the vibration source may vibrate at a single frequency, with a standard deviation that is less than 1%, 5%, or 10% of the single frequency. As another example, the vibration source may vibrate at a frequency randomly selected in a frequency bandwidth, e.g., 80-90 Hz. In some embodiments, the mechanical vibration source is configured to generate vibrating motions in one, two, or three dimensions. In some embodiments, the mechanical vibration source is configured to generate vibrating motions that includes translation, rotation, or both in two or three dimensions. In some embodiments, the mechanical vibration source is configured to but is not limited to generate linear or non-linear vibrations, random or deterministic vibrations, and/or undamped or damped vibrations.

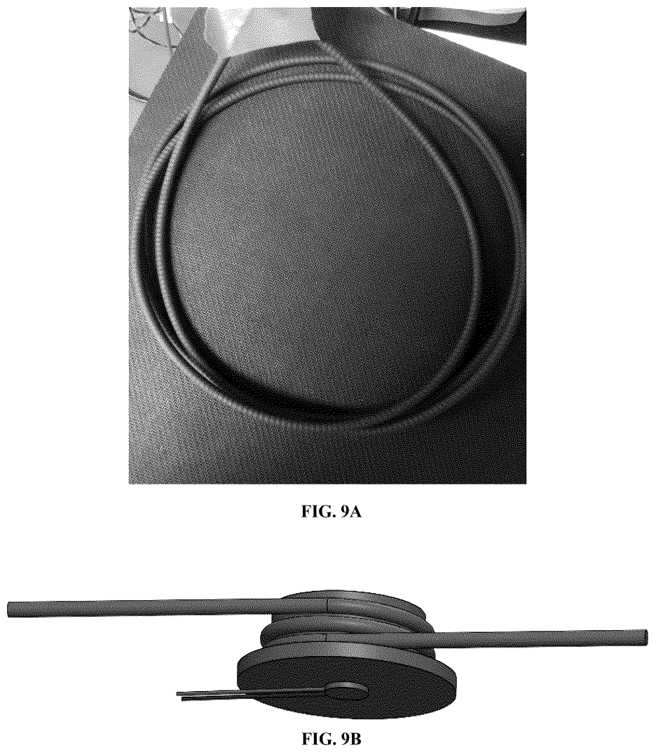

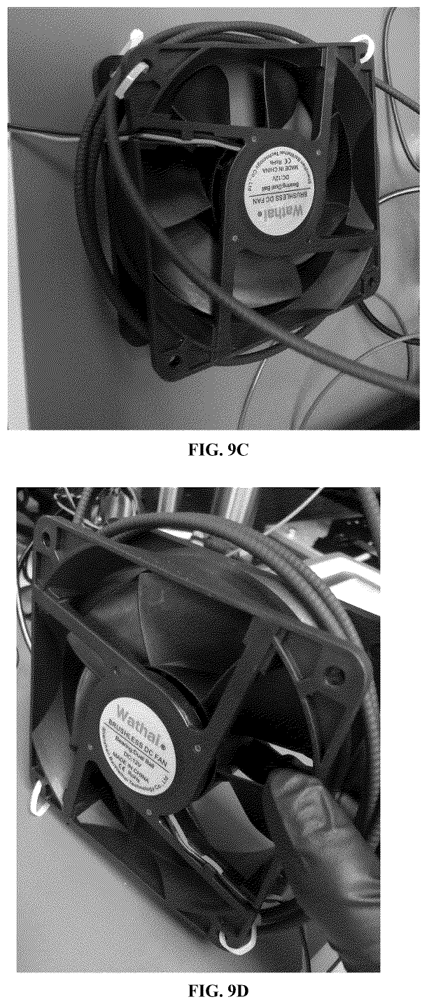

In some embodiments, at least a portion of the optical fiber(s) is wound or coiled for one or more rounds, as shown in FIGS. 9A-9D. The wound up or coiled up portion may include a radius that is no less than the minimum bend radius of the optical fiber to avoid damage to the optical fiber. For example, the wound or coiled up portion may include a radius of 60 mm, 65 mm, 70 mm or more with a minimum bend radius of 60 mm. The wound up or coiled up portion may be, but is not required to be, perfectly circular, as shown in FIGS. 9A-9D. In some embodiments, the wound or coiled up portion may include at least 2, 3, 4, 5, or more rounds. In some embodiments, the number of rounds of the wound or coiled up portion may be limited by the length of the optical fiber and the minimum bend radius of the optical fiber to enable a maximum possible number of rounds. In some embodiments, the number of rounds of the wound or coiled up portion may be maximized based on the length of the optical fiber and the minimum bend radius of the optical fiber. In some embodiments, at least a portion of the optical fiber at or near its ends is not wound up or coiled up. The non-coiled portion of the optical fiber at each end thereof may be less than 2%, 4%, 5%, 8%, or 10% of the total fiber length. The non-coiled portion of the optical fiber at each end thereof may be less than 0.05 m, 0.1 m, 0.15 m, 0.2 m, or 0.3 m.

In some embodiments, at least part of the optical fiber is loosely or fixedly attached to the mechanical vibration source. For example, the optical fiber can be taped to an off-the-shelf fan with or without wounding or coiling up for one or more rounds. The off-the-shelf fan may be the cooling fan for the CPU or other part(s) of the sequencing system described herein. As shown in FIGS. 9C-9D, the optical fiber is wound or coiled up around the off-the-shelf fan or further taped to the fan at some location(s). As shown in FIG. 9B, the fiber is coiled up on a wheel, and the vibration source is fixedly attached to a center of the wheel, at the bottom.

The mechanical vibration source can be various machineries that are capable of producing vibration motion(s). The mechanical vibration source can be various off-the-shelf machines that are much more cost-efficient than commercially available despecklers. As nonlimiting examples, the mechanical vibration source can include one or more of: an eccentric rotating mass (ERM) vibration motor, a linear resonant actuator, a coin vibration motor, a cell phone vibration motor, a sonic or ultrasonic vibration motor (e.g., such as one used for an electric toothbrush), an orbital gear or gear set, an orbital weight, etc.

In some embodiments, the despeckler is physically isolated from other elements of the imaging module, except the optical fiber, to minimize the influence of the despeckler's motion on sequencing reactions and/or imaging quality. For example, the despeckler is not coupled to the optical fiber at or near either end of the optical fiber, e.g., less than 0.1 m. As an example, the despeckler is not positioned within a pre-determined threshold distance from the other elements of the imaging module, e.g., at least 0.1 m, 0.2 m, 0.5 m, or more. In some embodiments, the despeckler is physically isolated from the sample stage, the objective lens, and/or the one or more image sensors, so that mechanical motion of the despeckler is independent from the sample state, the objective lens, and the one or more image sensors. As an example, the despeckler is at least 0.1 m, 0.2 m, 0.5 m, or more away from the sample stage, the objective lens, and/or the one or more image sensors. In some embodiments, the despeckler is configured to reduce speckle noise to be no more than 4%, 4.5%, 5%, or 5.5%. In some embodiments, the despeckler is configured to reduce speckle noise by at least 10%, 15%, 20%, 30%, 35%, 40% or more so that the speckle noise after despeckler reduction can be less than 40%, 50%, 55%, 60%, 65%, 70%, or 70% of the speckle noise before reduction.

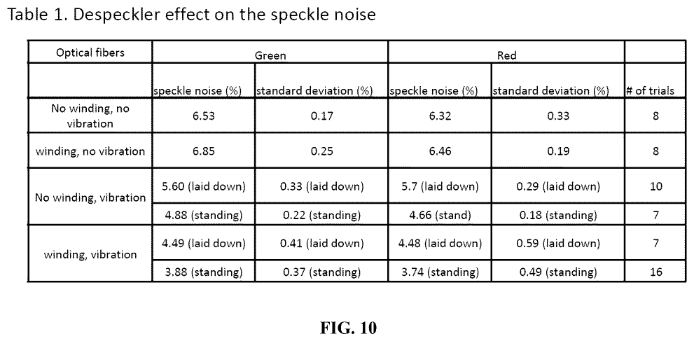

Table 1 in FIG. 10 shows the effect of different embodiments of the despecklers herein on the speckle noise in optical fibers. Two optical fibers that are each coupled to a green and red light source are examined. The speckle noise was reduced from 6.53% or 6.32% to less than 4.5% with winding of the optical fiber for 3 rounds and vibration of at least the wound-up portion by an off-the-shelf fan. The speckle reduction effect is better when keeping the fan in a standing position rather than having it laid down on a tabletop or an otherwise horizontal surface. FIGS. 9C-9D show the fan while it is standing or laid down.

In some embodiments, the speckle noise can be calculated using various methods that determine the level of uniformity of the optical beam, e.g., standard deviation of beam intensity at the sample plane. For example, a 2D beam profile within an imaging FOV can be separated into multiple regions, standard deviation of intensity can be determined for each individual region, and the average standard deviation across all the regions can be the speckle noise level.

In some embodiments, the despeckler is positioned in an optical path between a collimator and the objective lens of the imaging module, e.g. as shown in FIG. 6. The despeckler is configured to generate microscopic motion. The despeckler at such a larger beam location may advantageously avoid high light power density on the despeckler to avoid damage to the despeckler. In some embodiments, the despeckler here may include a single despeckler. In some embodiments, the despeckler here may include a combination of one or more first despecklers associated with the optical fiber(s) and one or more second large-beam despecklers positioned after the collimator but before the objective lens or sample plane in the optical path. In some embodiments, the large-beam despeckler is positioned where a maximum dimension (e.g., diameter or diagonal) of an optical beam at its cross section (orthogonal to the z axis) is greater than 1 mm, 2 mm, 5 mm, 8 mm, 10 mm, or 20 mm.

In an aspect, the present disclosure provides an optical system. The optical system can comprise a stage configured to hold a solid support (e.g., flow cell). The stage may be as described elsewhere herein. The optical system may comprise a light source configured to illuminate the solid support as described elsewhere herein. The optical system may comprise a despeckler optically coupled to the light source and disposed within an optical path from the light source to the stage.

The despeckler may be as described elsewhere herein. The despeckler may be configured to reduce speckle noise, act as a coupler for light sources, or a combination thereof. For example, the despeckler can be configured to receive light from multiple light sources and combine the light from the multiple light sources into a single light beam. In this example, a variety of excitation wavelengths can be combined to a single optical path and despeckled simultaneously. At least about 1, 2, 3, 4, 5, 6, 7, 8, 9, 10, or more light sources can be optically coupled to a single despeckler. In some cases, each light source can be optically coupled into a different despeckler. For example, four light sources can each be coupled into four despecklers, thereby despeckling the light from each light source.

The despeckler may comprise one or more of a diffuse despeckler (e.g., a despeckler comprising a diffuser such as a stationary diffuser, a rotational diffuser, etc.), a spatial light modulator, a phase despeckler (e.g., a despeckler configured to change the phase of the light), a polarization despeckler, a vibrational despeckler (e.g., a despeckler configured to vibrate one or more optical elements such as mirrors, fiber optics, lenses, etc.), a tension despeckler (e.g., a despeckler configured to modulate a tension of an optical fiber to induce despeckling) or the like, or any combination thereof. The despeckler may be configured to reduce speckle noise of the optical system by at least about 1, 2, 3, 4, 5, 6, 7, 8, 9, 10, 15, 20, 25, 30, 35, 40, 45, 50, 55, 60, 65, 70, 75, 80, 85, 90, 95, 99, or more percent. The despeckler may be configured to reduce speckle noise in the optical system to at most about 25, 20, 15, 14, 13, 12, 11, 10, 9, 8, 7, 6, 5, 4, 3, 2, 1, or less percent. Use of a despeckler can improve image quality of the optical system by reducing the speckle noise generated in the optical system, enhancing contrast or the like, or a combination thereof. Use of a vibrational despeckler may provide unexpected benefits to the optical system, as vibration may normally be avoided due to the effects of the vibration on the resolution of the optical system. By instead using vibration (e.g., vibration already present in the system from fans, motors, etc. already present in the system), the methods and system of the present disclosure can provide enhanced illumination profiles and imaging.

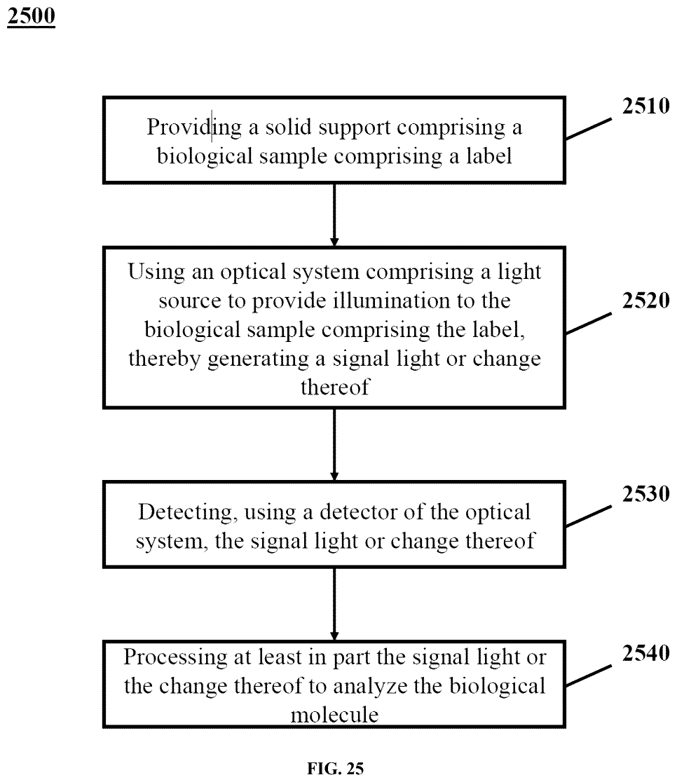

FIG. 25 shows a flow chart of a method 2500 for analyzing a biological sample, according to some embodiments. In an operation 2510, the method 2500 may comprise providing a solid support (e.g., flow cell) comprising the biological sample. The biological sample may comprise a label as described elsewhere herein. The biological sample may be as described elsewhere herein (e.g., the biological sample may comprise a nucleic acid molecule, a protein, a polypeptide, etc.). The label may be an optical label as described elsewhere herein. The biological sample may comprise a two-dimensional biological sample. The biological sample may comprise a three-dimensional biological sample.

In another operation 2520, the method 2500 may comprise using an optical system comprising a light source to provide illumination to the biological sample comprising the label, thereby generating a signal light or a change thereof. The optical system may be as described elsewhere herein. For example, the optical system may comprise a despeckler. The illumination may be provided through the despeckler oriented in an optical path of the optical system. The despeckler may be as described elsewhere herein. For example, the despeckler may be a vibrational despeckler. In some cases, an additional light source can be used to illuminate the solid support. The additional light source may provide a different wavelength of light to the flow cell from the light source. For example, the light source can provide a first wavelength configured to excite a first label, and the additional light source can provide a second wavelength configured to excite a second label. The additional light source may be optically coupled to the despeckler. For example, the light source and the additional light source can both be optically coupled to the same despeckler, and the output of the despeckler can comprise light from both light sources.

In another operation 2530, the method 2500 may comprise detecting, using a detector of the optical system, the signal light or the change thereof. The detecting may be as described elsewhere herein. For example, the detecting may comprise directing the signal light or change thereof to the detector using an optical assembly. In some cases, the detecting may comprise time-gated detecting.

In another operation 2540, the method 2500 may comprise processing at least in part the signal light or the change thereof to analyze the biological sample. The processing may comprise use of one or more computer systems as described elsewhere herein. The processing may comprise generating one or more base calls of the sample.

In some cases, the method 2500 may comprise repeating operations 2520-2540 for an additional biological sample coupled to an additional surface of the solid support. For example, a plurality of biological molecules can be coupled to the solid support with a plurality of labels each affixed to the plurality of biological molecules, and the optical system can image each of the labels.

In some cases, the method 2500 may comprise repeating operations 2510-2540 for an additional label that binds to another portion of the biological molecule. For example, a first label can identify a first nucleotide of a nucleic acid, the first nucleotide can be removed from the nucleic acid, and a second label can be hybridized to the nucleic acid molecule at a second nucleotide. In this example, the second label can be identified in a similar way to the first, providing information related to the second nucleotide of the nucleic acid molecule.

### Light Beam Delivery Subsystems

The light beam delivery subsystems herein may include one or more collimators and one or more optical lens elements. In some embodiments, the power existing the light beam delivery subsystem is greater than 5, 8, 10, 12, or 14 Watts for one or more wavelengths or wavelength ranges. In some embodiments, a power at a sample plane is greater than 5, 8, 10, 12, or 14 Watts for one or more wavelengths or wavelength ranges. In some embodiments, a power existing the light beam delivery subsystem is greater than 5, 8, 10, 12, or 14 Watts for one or more colors. In some embodiments, a power at a sample plane is greater than 5, 8, 10, 12, or 14 Watts for one or more colors.

In some embodiments, the one or more collimators may be spaced apart along the z-axis or in the x-y plane. In some embodiments, the light beam delivery system may include a single collimator. FIGS. 1 and 6 shows examples of embodiments of the light beam delivery subsystem. As shown in FIG. 1, the one or more optical lens elements comprise one or more multi-lens arrays (e.g., MLA1 and MLA2). In some embodiments, each multi-lens array comprises one or more of: an asymmetric convex-convex lens, a convex-plano lens, a concave-plano lens, an asymmetric concave-concave lens, and an asymmetric convex-concave lens. Each of the multi-lens arrays may comprise multiple lens elements at least in a direction that is orthogonal to a z-axis. For example, the multiple lens elements of the array can be distributed along the x or y-axis or any direction in a x-y plane orthogonal to the z-axis (FIG. 1). In some embodiments, the one or more optical lens elements comprise: an asymmetric convex-convex lens, a convex-plano lens, a concave-plano lens, an asymmetric concave-concave lens, and an asymmetric convex-concave lens, or a combination thereof.

In some embodiments, the one or more optical lens elements comprise: a first multi-lens array (MLA1) and a second multi-lens array (MLA2) that are positioned along a z-axis between the collimator and an entrance pupil of the illumination system, as shown in FIG. 1. In this particular embodiment, the illumination system includes a wide-field illumination module with fiber-coupled diode laser inputs interleaved at an angle on adjacent columns on the first multi-element lens array pair, MLA1 and MLA2. Multiple images of the light source are created in the external or entrance pupil to form a uniform illumination field at the sample field, e.g., the flow cell. The illumination system can comprise a fiber coupled laser diode. The illuminator design is based on generating multiple source images in the entrance pupil plane of the imaging module. The output of the fiber-couple laser is optically connected to a collimator and then segmented in pupil space by a number of multi-element lens arrays to form these secondary illumination sources. The imaging group, G3, in FIG. 1, is a surrogate for the imaging module between the illumination system and the sample(s). The imaging group G3 can be shared by the imaging and illumination optical paths. In some embodiments, the illumination system is configured to generate secondary illumination sources that overlap at the sample stage or sample positioned thereon, thus averaging the individual intensities to provide intensity with improved uniformity compared to some methods.

In some embodiments, various laser-based illumination sources may suffer from speckle and that speckle intensity may be controlled. The source coherent (e.g., speckle) may be mitigated by using a 1-3 m length of fiber with a 200-800 um core. In some embodiments, the illumination system may allow mode mixing to reduce speckle to prespecified levels. In some embodiment, a time-variant diffuser reduction method may be integrated into the beam path to improve the source coherency. FIG. 3A-3C illustrates the intensity profiles at the sample using the illumination system herein.

### Sequencing Systems

FIG. 11 illustrates a block diagram of a system 100 for imaging sequencing reactions of sample(s) on a flow cell, according to an embodiment. The system 100 has a sequencing system 110 that may include a flow cell 112, a sequencer 114, an imager (e.g., optical system) 116, data storage 122, and user interface 124. The sequencing system 110 may be connected to a cloud 130. The sequencing system 110 may include one or more of dedicated processors 118, Field-Programmable Gate Array(s) (FPGA(s)) 120, and a computer system 126.

In some embodiments, the flow cell 112 is configured to capture DNA fragments and form DNA sequences for base-calling on the flow cell. The flow cell 112 can include a support as disclosed herein. The support can be a solid support. The support can include a surface coating thereon as disclosed herein. The surface coating can be a polymer coating as disclosed herein.

A flow cell 112 can include multiple tiles or otherwise imaging areas thereon, and each tile may be separated into a grid of subtiles. Each subtile can include a plurality of clusters or polonies thereon. As a nonlimiting example, a flow cell can have 424 tiles, and each tile can be divided into a 6×9 grid, therefore 54 subtiles.

The flow cell images herein are images of a sample immobilized on a support, e.g., a flow cell. The flow cell image as disclosed herein can be an image including signals of a plurality of clusters or polonies. The flow cell image can include one or more tiles of signals or one or more subtiles of signals. In some embodiments, a flow cell image can be an image that includes all the tiles and approximately all signals thereon. The flow cell image can be acquired from a channel during an imaging or sequencing cycle using the imager 116. In some embodiments, each tile may include millions of polonies or clusters. As a nonlimiting example, a tile can include about 1 to 10 million of clusters or polonies. Each polony can be a collection of many copies of DNA fragments. The clusters or polonies may appear as bright spot expanding from less than a pixel to a couple of pixels.

The flow cell images may be of various sizes or field-of-views (FOVs). In some embodiments, each of the flow cell images comprises a wide field-of-view (FOV) that is greater than 20 mm², 30 mm², 40 mm², or 50 mm². In some embodiments, each of the flow cell images comprises a field-of-view (FOV) that completely overlaps with or contained within the illumination field generated by the illumination system in the sample plane. In some embodiments, each of the flow cell images comprises a field-of-view (FOV) that overlaps with at least 80%, 85%, 90%, or 95% of an illumination field generated by the illumination system at the sample plane. In some embodiments, each of the flow cell images comprises a field-of-view (FOV) with a size that is at least 80%, 85%, 90%, or 95% of the size of the illumination field generated by the illumination system at the sample plane.

In some embodiments, to capture such wide FOVs, the image sensor may be of a wider size than other image sensors for some NGS sequencing systems. In some embodiments, the image sensor may include a sensor size that is greater than 20 mm², 30 mm², 40 mm², or 50 mm². In some NGS systems, the flow cell images are generally in a range from 1 mm² to 8 mm². As a result, an illumination field that is greater than 10 mm² is not preferred in these NGS systems to avoid or reduce undesired photon bleaching in neighboring areas to the image FOVs. The illumination system herein generates an illumination field that is 2×, 5×, 10×, or larger than the illumination fields in these NGS systems. In some embodiments, the imager 116 is capable of generating FOVs of flow cell images that is comparable to the size of the wide-illumination field herein. The FOVs and the illumination field may be customized so that they overlap or substantially overlap with each other. Additionally, the illumination field may be customized so that its shape can match the shape of the FOV to facilitate such overlap. With such overlap, photon bleaching in the unimaged area of the sample(s) caused by a illumination field wider than the FOV of the flow cell images is avoided or minimized.

The sequencer 114 may be configured to flow a nucleotide mixture onto the flow cell 112, cleave blockers from the nucleotides in between flowing steps, and perform other steps for the formation of the DNA sequences on the flow cell 112. The nucleotides may have fluorescent elements attached that emit light or energy in a wavelength that indicates the type of nucleotide. Each type of fluorescent element may correspond to a particular nucleotide base (e.g., A, G, C, T). The fluorescent elements may emit light in visible wavelengths.

For example, each nucleotide base may be assigned a color. In some cases, adenine may be red, cytosine may be blue, guanine may be green, and thymine may be yellow. The color or wavelength of the fluorescent element for each nucleotide may be selected so that the nucleotides are distinguishable from one another based on the wavelengths of light emitted by the fluorescent elements.

The imager 116 may be configured to capture images of the flow cell 112 after each flowing step. The imager 116 may include one or more imaging modules disclosed herein. For example, the imager may include an optical system comprising 4 different imaging modules, each for capturing flow cell images from a different color channel.

In an embodiment, the imager 116 includes a camera configured to capture digital images, such as a CMOS or a CCD camera. The camera may be configured to capture images at the wavelengths of the fluorescent elements bound to the nucleotides.

The resolution of the imager 116 controls the level of detail in the flow cell images, including pixel size. In existing systems, this resolution is very important, as it controls the accuracy with which a spot-finding algorithm identifies the cluster centers. One way to increase the accuracy of spot finding is to improve the resolution of the imager 116, or improve the processing performed on images taken by the imager 116. The methods described herein may detect cluster centers in pixels other than those detected by a spot-finding algorithm. These methods allow for improved accuracy in detection of cluster centers without increasing the resolution of the imager 116. The resolution of the imager may even be less than existing systems with comparable performance, which may reduce the cost of the sequencing system 110.

In an embodiment, the images of the flow cell may be captured in groups, where each image in the group is taken at a wavelength or in a spectrum that matches or includes only one of the fluorescent elements. In another embodiment, the images may be captured as single images that capture at least a portion (e.g., a portion, all, etc.) of the wavelengths of the fluorescent elements.

The sequencing system 100 may be configured to identify cluster locations on the flow cell 112 based on the flow cell images. The processing for identifying the cluster may be performed by the dedicated processors 118, the FPGA(s) 120, the computing system 126, or a combination thereof. Identifying or determining the cluster locations may involve performing traditional cluster finding in combination with the cluster finding methods described more particularly herein.

General purpose processors provide interfaces to run a variety of program in an operating system, such as Windows™ or Linux™. Such an operating system can provides great flexibility to a user.

In some embodiments, the dedicated processors 118 may be configured to perform operations of the cluster finding methods described herein. They may not be general-purpose processors, but instead custom processors with specific hardware or instructions for performing those operations. Dedicated processors directly run specific software without an operating system.

The lack of an operating system reduces overhead, at the cost of the flexibility in what the processor may perform. A dedicated processor may make use of a custom programming language, which may be designed to operate more efficiently than the software run on general-purpose processors. This may increase the speed at which the operations are performed and allow for real time processing.

In some embodiments, the FPGA(s) 120 may be configured to perform operations of the cluster finding methods described herein. An FPGA is programmed as hardware that will only perform a specific task. A special programming language may be used to transform software operations into hardware componentry. Once an FPGA is programmed, the hardware directly processes digital data that is provided to it without running software. The FPGA instead uses logic gates and registers to process the digital data. Because there is no overhead required for an operating system, an FPGA generally processes data faster than a general purpose processor. Similar to dedicated processors, this is at the cost of flexibility.

The lack of software overhead may also allow an FPGA to operate faster than a dedicated processor, although this will depend on the exact processing to be performed and the specific FPGA and dedicated processor.

A group of FPGA(s) 120 may be configured to perform the operations in parallel. For example, a number of FPGA(s) 120 may be configured to perform a processing operation for an image, a set of images, or a cluster location in one or more images. Each FPGA(s) 120 may perform its own part of the processing operation at the same time, reducing the time needed to process data. This may allow the processing operations to be completed in real time. Further discussion of the use of FPGAs is provided below.

Compared to methods that may need to store the data before it can be processed, which may require more memory or accessing a computer system located in the cloud 130, performing the processing steps in real time may allow the system to use less memory, as the data may be processed as it is received.

In some embodiments, the data storage 122 is used to store information used in the identification of the cluster locations. This information may include the images themselves or information derived from the images captured by the imager 116. The DNA sequences determined from the base-calling may be stored in the data storage 122. Parameters identifying cluster locations may also be stored in the data storage 122.

The user interface 124 may be used by a user to operate the sequencing system or access data stored in the data storage 122 or the computer system 126.

The computer system 126 may control the general operation of the sequencing system and may be coupled to the user interface 124. It may also perform operations in the identification of cluster locations and base-calling. In some embodiments, the computer system 126 is a computer system. The computer system 126 may store information regarding the operation of the sequencing system 110, such as configuration information, instructions for operating the sequencing system 110, or user information. The computer system 126 may be configured to pass information between the sequencing system 110 and the cloud 130.

As discussed above, the sequencing system 110 may have dedicated processors 118, FPGA(s) 120, or the computer system 126. The sequencing system may use one, two, or all of these elements to accomplish necessary processing described above. In some embodiments, when these elements are present together, the processing tasks are split between them. For example, the FPGA(s) 120 may be used to perform the cluster center finding methods described herein, while the computer system 126 may perform other processing functions for the sequencing system 110.

Various combinations of these elements can allow various system embodiments that balance efficiency and speed of processing with cost of processing elements.

The cloud 130 may be a network, remote storage, or some other remote computing system separate from the sequencing system 110. The connection to cloud 130 may allow access to data stored externally to the sequencing system 110 or allow for updating of software in the sequencing system 110.

### Supports and Low Non-Specific Coatings

In some embodiments, NGS sequencing compositions and methods, e.g., pairwise sequencing, employ a support comprising a plurality of oligonucleotide surface primers immobilized thereon. In some embodiments, the support is passivated with a low non-specific binding coating. The surface coatings described herein exhibit very low non-specific binding to reagents typically used for nucleic acid capture, amplification, and sequencing workflows, such as dyes, nucleotides, enzymes, and nucleic acid primers. The surface coatings exhibit low background fluorescence signals or high contrast-to-noise (CNR) ratios compared to some surface coatings.

The low non-specific binding coating comprises one layer or multiple layers (FIG. 11). In some embodiments, the plurality of surface primers is immobilized to the low non-specific binding coating. In some embodiments, at least one surface primer is embedded within the low non-specific binding coating. The low non-specific binding coating enables improved nucleic acid hybridization and amplification performance. In general, the supports comprise a substrate (or support structure), one or more layers of a covalently or non-covalently attached low-binding, chemical modification layer such as silane layers or polymer films, and one or more covalently or non-covalently attached surface primers that can be used for tethering single-stranded nucleic acid library molecules to the support. In some embodiments, the formulation of the coating, e.g., the chemical composition of one or more layers, the coupling chemistry used to cross-link the one or more layers to the support and/or to each other, and the total number of layers, may be varied such that non-specific binding of proteins, nucleic acid molecules, and other hybridization and amplification reaction components to the coating is minimized or reduced relative to a comparable monolayer. The formulation of the coating described herein may be varied such that non-specific hybridization on the coating is minimized or reduced relative to a comparable monolayer. The formulation of the coating may be varied such that non-specific amplification on the coating is minimized or reduced relative to a comparable monolayer. The formulation of the coating may be varied such that specific amplification rates and/or yields on the coating are maximized. Amplification levels suitable for detection are achieved in no more than 2, 3, 4, 5, 6, 7, 8, 9, 10, 15, 20, 25, 30, or more than 30 amplification cycles in some cases disclosed herein.

The support structure that comprises the one or more chemically-modified layers, e.g., layers of a low non-specific binding polymer, may be independent or integrated into another structure or assembly. For example, in some embodiments, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments, the support structure comprises the interior surface (such as the lumen surface) of a capillary etched into a planar chip.

The attachment chemistry used to graft a first chemically-modified layer to the surface of the support will generally be dependent on both the material from which the surface is fabricated and the chemical nature of the layer. In some embodiments, the first layer may be covalently attached to the surface. In some embodiments, the first layer may be non-covalently attached, e.g., adsorbed to the support through non-covalent interactions such as electrostatic interactions, hydrogen bonding, or van der Waals interactions between the support and the molecular components of the first layer. In either case, the support may be treated prior to attachment or deposition of the first layer. A variety of surface preparation techniques may be used to clean or treat the surface. For example, glass or silicon surfaces may be acid-washed using a Piranha solution (a mixture of sulfuric acid H₂SO₄ and hydrogen peroxide H₂O₂), base treated with KOH and NaOH, and/or cleaned using an oxygen plasma treatment method.

Silane chemistries constitute non-limiting approaches for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, or C18 hydrocarbons, or linear polyethylene glycol (PEG) molecules) or layer molecules (e.g., branched PEG molecules or other polymers) to the surface. Examples of suitable silanes that may be used in creating any of the disclosed low binding coatings include, but are not limited to, (3-Aminopropyl) trimethoxysilane (APTMS), (3-Aminopropyl)triethoxysilane (APTES), any of a variety of PEG-silanes (e.g., comprising molecular weights of 1K, 2K, 5K, 10K, 20K, etc.), amino-PEG silane (e.g., comprising a free amino functional group), maleimide-PEG silane, biotin-PEG silane, and the like.

A variety of molecules including, but not limited to, amino acids, peptides, nucleotides, oligonucleotides, other monomers or polymers, or combinations thereof may be used in creating the one or more chemically-modified layers on the support, where the choice of components used may be varied to alter one or more properties of the layers such as the surface density of functional groups and/or tethered oligonucleotide primers, the hydrophilicity/hydrophobicity of the layers, or the three three-dimensional nature (e.g., “thickness”) of the layers. Examples of polymers that may be used to create one or more layers of low non-specific binding material in any of the disclosed coatings include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), His tag-Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The low non-specific binding surface coating may be applied uniformly across the support. Alternatively, the surface coating may be patterned, such that the chemical modification layers are confined to one or more discrete regions of the support. For example, the coating may be patterned using photolithographic techniques to create an ordered array or random pattern of chemically-modified regions on the support. Alternately or in combination, the coating may be patterned using, e.g., contact printing and/or ink-jet printing techniques. In some embodiments, an ordered array or random pattern of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In some embodiments, the low nonspecific binding coatings comprise hydrophilic polymers that are non-specifically adsorbed or covalently grafted to the support. Passivation may be performed utilizing poly(ethylene glycol) (PEG, also known as polyethylene oxide (PEO) or polyoxyethylene) or other hydrophilic polymers with different molecular weights and end groups that are linked to a support using, for example, silane chemistry. The end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface.

In some embodiments, two or more layers may be covalently coupled to each other or internally cross-linked to improve the stability of the resulting coating. In some embodiments, surface primers with different nucleotide sequences and/or base modifications (or other biomolecules, e.g., enzymes or antibodies) may be tethered to the resulting layer at various surface densities. In some embodiments, for example, both surface functional group density and surface primer concentration may be varied to attain a specified surface primer density range. Additionally, surface primer density can be controlled by diluting the surface primers with other molecules that carry the same functional group. For example, amine-labeled surface primers can be diluted with amine-labeled polyethylene glycol in a reaction with an NHS-ester coated surface to reduce the final primer density. Surface primers with different lengths of linker between the hybridization region and the surface attachment functional group can also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

In some embodiments, the low nonspecific binding coatings comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl)acrylamide-co-acrylamide (PAZAM).

In order to scale primer surface density and add additional dimensionality to hydrophilic or amphoteric coatings, supports comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the support significantly. Some PEG coating approaches use monolayer primer deposition, which have been generally reported for single molecule applications but do not yield high copy numbers for nucleic acid amplification applications. As described herein, “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some embodiments, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some embodiments, high primer density materials may be constructed in solution and subsequently layered onto the surface in multiple steps.

Examples of materials from which the support structure may be fabricated include, but are not limited to, glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic support structures are contemplated.

The support structure may be rendered in a variety of geometries and dimensions, and may comprise a variety of materials. For example, the support structure may be locally planar (e.g., comprising a microscope slide or the surface of a microscope slide). Globally, the support structure may be cylindrical (e.g., comprising a capillary or the interior surface of a capillary), spherical (e.g., comprising the outer surface of a non-porous bead), or irregular (e.g., comprising the outer surface of an irregularly-shaped, non-porous bead or particle). In some embodiments, the surface of the support structure used for nucleic acid hybridization and amplification may be a solid, non-porous surface. In some embodiments, the surface of the support structure used for nucleic acid hybridization and amplification may be porous, such that the coatings described herein penetrate the porous surface, and nucleic acid hybridization and amplification reactions performed thereon may occur within the pores.

The support structure that comprises the one or more chemically-modified layers, e.g., layers of a low non-specific binding polymer, may be independent or integrated into another structure or assembly. For example, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments the support structure comprises the interior surface (such as the lumen surface) of a capillary etched into a planar chip.

As noted, the low non-specific binding supports of the present disclosure exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, exposure of the surface to fluorescent dyes (e.g., cyanins such as Cy3, or Cy5, etc., fluoresceins, coumarins, rhodamines, etc. or other dyes disclosed herein), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging, may be used as a qualitative tool for comparison of non-specific binding on supports comprising different surface formulations. In some embodiments, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging, may be used as a quantitative tool for comparison of non-specific binding on supports comprising different surface formulations. Care can be taken to ensure that the fluorescence imaging is performed under conditions where fluorescence signal is linearly related (or related in a predictable manner) to the number of fluorophores on the support surface (e.g., under conditions where signal saturation and/or self-quenching of the fluorophore is not an issue) and suitable calibration standards are used. In some embodiments, other techniques such as radioisotope labeling and counting methods may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed by detection of the amount of label remaining on the surface and comparison of the signal resulting therefrom to an appropriate calibration standard. In some embodiments, the label may comprise a fluorescent label. In some embodiments, the label may comprise a radioisotope. In some embodiments, the label may comprise any other detectable label. In some embodiments, the degree of non-specific binding exhibited by a given support surface formulation may thus be assessed in terms of the number of non-specifically bound protein molecules (or nucleic acid molecules or other molecules) per unit area. In some embodiments, the low-binding supports of the present disclosure may exhibit non-specific protein binding (or non-specific binding of other specified molecules, (e.g., cyanins such as Cy3, or Cy5, etc., fluoresceins, coumarins, rhodamines, etc. or other dyes disclosed herein)) of less than 0.001 molecule per m², less than 0.01 molecule per m², less than 0.1 molecule per m², less than 0.25 molecule per m², less than 0.5 molecule per m², less than 1 molecule per m², less than 10 molecules per m², less than 100 molecules per m², or less than 1,000 molecules per μm². A given support surface of the present disclosure may exhibit non-specific binding falling anywhere within this range, for example, of less than 86 molecules per μm².

For example, some modified surfaces disclosed herein exhibit nonspecific protein binding of less than 0.5 molecule/am² following contact with a 1 μM solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm². In independent nonspecific binding assays, 1 μM labeled Cy3 SA (ThermoFisher), 1 μM Cy5 SA dye (ThermoFisher), 10 μM Aminoallyl-dUTP-ATTO-647N (Jena Biosciences), 10 μM Aminoallyl-dUTP-ATTO-Rhol 1 (Jena Biosciences), 10 M Aminoallyl-dUTP-ATTO-Rhol 1 (Jena Biosciences), 10 μM 7-Propargylamino-7-deaza-dGTP-Cy5 (Jena Biosciences, and 10 μM 7-Propargylamino-7-deaza-dGTP-Cy3 (Jena Biosciences) were incubated on the low binding coated supports at 37° C. for 15 minutes in a 384 well plate format. Each well was rinsed 2-3× with 50 ul deionized RNase/DNase Free water and 2-3× with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 m. For higher resolution imaging, images were collected on an Olympus IX83 microscope (e.g., inverted fluorescence microscope) (Olympus Corp., Center Valley, Pa.) with a total internal reflectance fluorescence (TIRF) objective (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, N.Y.), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per m². In some embodiments, the coated support was immersed in a buffer (e.g., 25 mM ACES, pH 7.4) while the image was acquired.

In some embodiments, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some embodiments, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some embodiments, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, for example, an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. A given hydrophilic, low-binding support surface of the present disclosure may exhibit a water contact angle having a value of anywhere within this range.

In some embodiments, the hydrophilic surfaces disclosed herein facilitate reduced wash times for bioassays, often due to reduced nonspecific binding of biomolecules to the low-binding surfaces. In some embodiments, adequate wash steps may be performed in less than 60, 50, 40, 30, 20, 15, 10, or less than 10 seconds. For example, adequate wash steps may be performed in less than 30 seconds.

Some low-binding surfaces of the present disclosure exhibit significant improvement in stability or durability to prolonged exposure to solvents and elevated temperatures, or to repeated cycles of solvent exposure or changes in temperature. For example, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some embodiments, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some embodiments, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some embodiments, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100-fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100-fold greater than a signal of an adjacent amplified nucleic acid population region of the surface.

In some embodiments, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

One or more types of primer may be attached or tethered to the support surface. In some embodiments, the one or more types of adapters or primers may comprise spacer sequences, adapter sequences for hybridization to adapter-ligated target library nucleic acid sequences, forward amplification primers, reverse amplification primers, sequencing primers, and/or molecular barcoding sequences, or any combination thereof. In some embodiments, 1 primer or adapter sequence may be tethered to at least one layer of the surface. In some embodiments, at least 2, 3, 4, 5, 6, 7, 8, 9, 10, or more than 10 different primer or adapter sequences may be tethered to at least one layer of the surface.

In some embodiments, the tethered adapter and/or primer sequences may range in length from about 10 nucleotides to about 100 nucleotides. In some embodiments, the tethered adapter and/or primer sequences may be at least 10, at least 20, at least 30, at least 40, at least 50, at least 60, at least 70, at least 80, at least 90, or at least 100 nucleotides in length. In some embodiments, the tethered adapter and/or primer sequences may be at most 100, at most 90, at most 80, at most 70, at most 60, at most 50, at most 40, at most 30, at most 20, or at most 10 nucleotides in length.

Any of the lower and upper values described in this paragraph may be combined to form a range included within the present disclosure, for example, in some embodiments the length of the tethered adapter and/or primer sequences may range from about 20 nucleotides to about 80 nucleotides. The length of the tethered adapter and/or primer sequences may have any value within this range, e.g., about 24 nucleotides.

In some embodiments, the resultant surface density of primers (e.g., capture primers) on the low binding support surfaces of the present disclosure may range from about 100 primer molecules per m² to about 100,000 primer molecules per m². In some embodiments, the resultant surface density of primers on the low binding support surfaces of the present disclosure may range from about 1,000 primer molecules per m² to about 1,000,000 primer molecules per μm². In some embodiments, the surface density of primers may be at least 1,000, at least 10,000, at least 100,000, or at least 1,000,000 molecules per m². In some embodiments, the surface density of primers may be at most 1,000,000, at most 100,000, at most 10,000, or at most 1,000 molecules per m². Any of the lower and upper values described in this paragraph may be combined to form a range included within the present disclosure, for example, in some embodiments the surface density of primers may range from about 10,000 molecules per m² to about 100,000 molecules per m². The surface density of primer molecules may have any value within this range, e.g., about 455,000 molecules per m². In some embodiments, the surface density of target library nucleic acid sequences initially hybridized to adapter or primer sequences on the support surface may be less than or equal to that indicated for the surface density of tethered primers. In some embodiments, the surface density of clonally-amplified target library nucleic acid sequences hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000/μm², while also comprising at least a second region having a substantially different local density.

### Contrast to Noise Ratio (CNR)

In some embodiments, the performance of nucleic acid hybridization, amplification reactions, or a combination thereof using the disclosed reaction formulations and low-binding supports may be assessed using fluorescence imaging techniques, where the contrast-to-noise ratio (CNR) of the images provides a key metric in assessing amplification specificity and non-specific binding on the support. CNR is defined as: CNR=(Signal−Background)/Noise. The background term is taken to be the signal measured for the interstitial regions surrounding a particular feature (diffraction limited spot, DLS) in a specified region of interest (ROI). While signal-to-noise ratio (SNR) is often considered to be a benchmark of overall signal quality, it can be shown that improved CNR can provide a significant advantage over SNR as a benchmark for signal quality in applications that require rapid image capture (e.g., sequencing applications for which cycle times must be minimized), as shown in the example below. At high CNR the imaging time required to reach accurate discrimination (and thus accurate base-calling in the case of sequencing applications) can be drastically reduced even with moderate improvements in CNR. Improved CNR in imaging data on the imaging integration time provides a method for more accurately detecting features such as clonally-amplified nucleic acid colonies on the support surface.

In some ensemble-based sequencing approaches, the background term may be measured as the signal associated with ‘interstitial’ regions. In addition to “interstitial” background (Binter), “intrastitial” background (Bintra) exists within the region occupied by an amplified DNA colony. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Binter background signal arises from a variety of sources; a few examples include auto-fluorescence from consumable flow cells, non-specific adsorption of detection molecules that yield spurious fluorescence signals that may obscure the signal from the ROI, and the presence of non-specific DNA amplification products (e.g., those arising from primer dimers). In some next generation sequencing (NGS) applications, this background signal in the current field-of-view (FOV) is averaged over time and subtracted. The signal arising from individual DNA colonies (e.g., (Signal)-B(interstial) in the FOV) yields a discernable feature that can be classified. In some embodiments, the intrastitial background (B(intrastitial)) can contribute a confounding fluorescence signal that is not specific to the target of interest, but is present in the same ROI thus making it far more difficult to average and subtract.

Nucleic acid amplification on the low-binding coated supports described herein may decrease the B(interstitial) background signal by reducing non-specific binding, may lead to improvements in specific nucleic acid amplification, and may lead to a decrease in non-specific amplification that can impact the background signal arising from both the interstitial and intrastitial regions. Compared to some methods, in some embodiments, the disclosed low-binding coated supports, optionally used in combination with the disclosed hybridization and/or amplification reaction formulations, may lead to improvements in CNR by a factor of 2, 5, 10, 100, 250, 500 or 1000-fold. Although described here in the context of using fluorescence imaging as the read-out or detection mode, the same principles apply to the use of the disclosed low-binding coated supports and nucleic acid hybridization and amplification formulations for other detection modes as well, including both optical and non-optical detection modes.

### Methods for Sequencing

The present disclosure provides methods for autofocusing optical systems that can be used for sequencing immobilized or non-immobilized template nucleotide acid molecules. In some embodiments, the immobilized template molecules comprise a plurality of nucleic acid template molecules having one copy of a target sequence of interest. In some embodiments, nucleic acid template molecules having one copy of a target sequence of interest can be generated by conducting bridge amplification using linear library molecules. In some embodiments, the immobilized template molecules comprise a plurality of nucleic acid template molecules each having two or more tandem copies of a target sequence of interest (e.g., concatemers). In some embodiments, nucleic acid template molecules comprising concatemer molecules can be generated by conducting rolling circle amplification of circularized linear library molecules. In some embodiments, the non-immobilized template molecules comprise circular molecules. In some embodiments, methods for sequencing employ soluble (e.g., non-immobilized) sequencing polymerases or sequencing polymerases that are immobilized to a support.

In some embodiments, the sequencing reactions employ detectably labeled nucleotide analogs. In some embodiments, the sequencing reactions employ a two-stage sequencing reaction comprising binding detectably labeled multivalent molecules and incorporating nucleotide analogs. In some embodiments, the sequencing reactions employ non-labeled nucleotide analogs. In some embodiments, the sequencing reactions employ phosphate chain-labeled nucleotides.

### Multivalent Molecules

The present disclosure provides methods for autofocusing optical systems that can be used for sequencing template nucleic acid molecules. In some embodiments, the sample immobilized or otherwise positioned on the support may include at least one multivalent molecule. In some embodiments, the sample that is used for autofocusing the optical system may include at least one multivalent molecule. In some embodiments, the sequencing methods utilizing the optical system for imaging may employ at least one multivalent molecule. In some embodiments, the sequencing methods utilizing the optical system for imaging may include autofocusing of the optical system before imaging one or more surfaces in a flow cycle of the sequencing run.

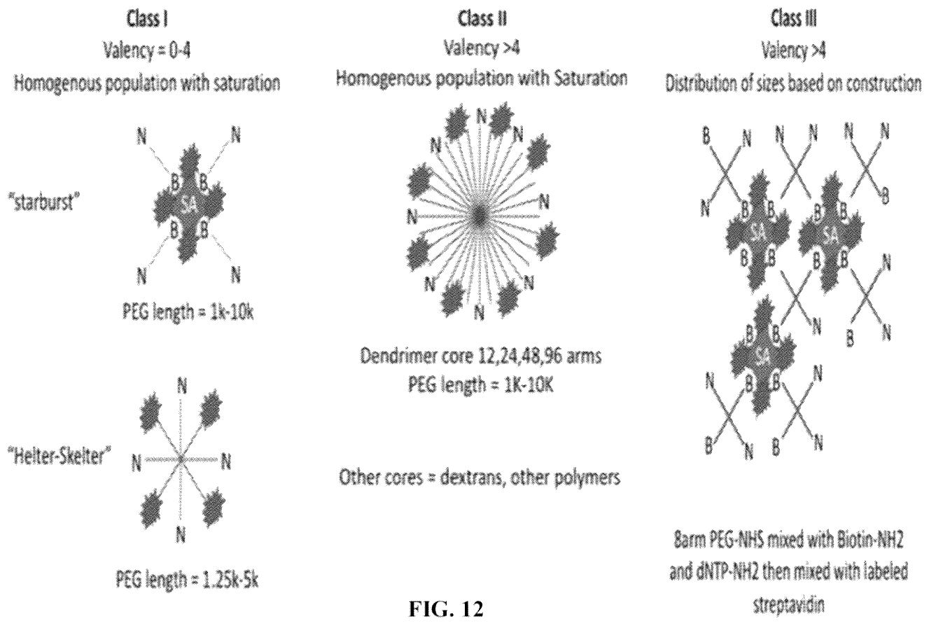

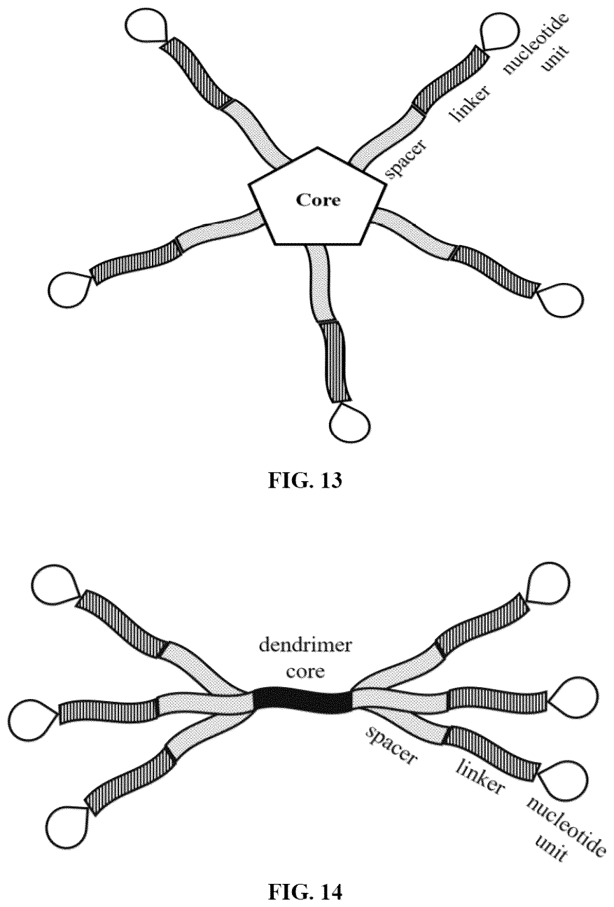

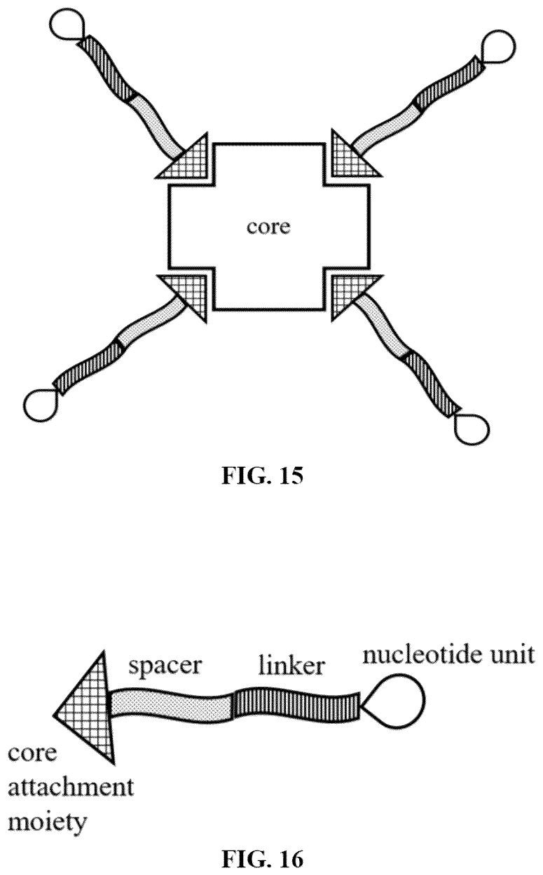

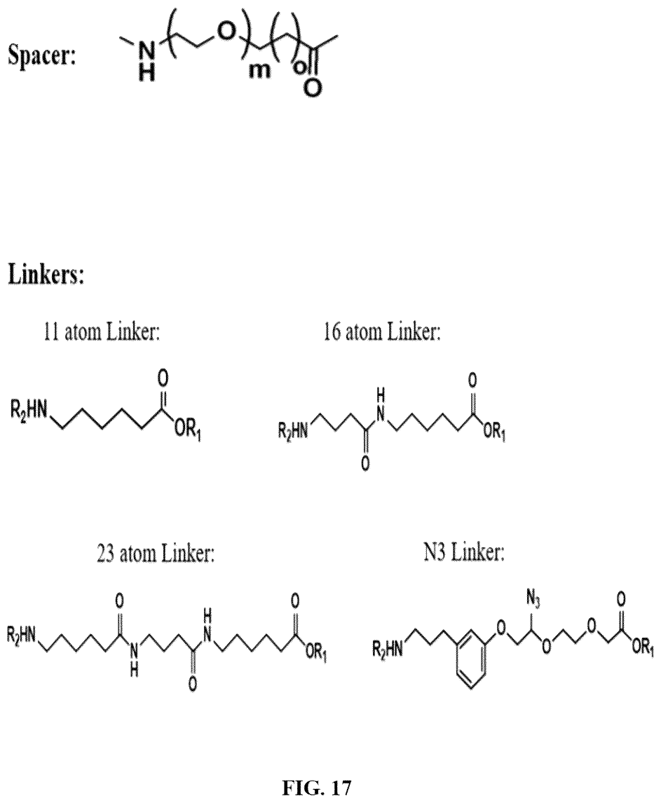

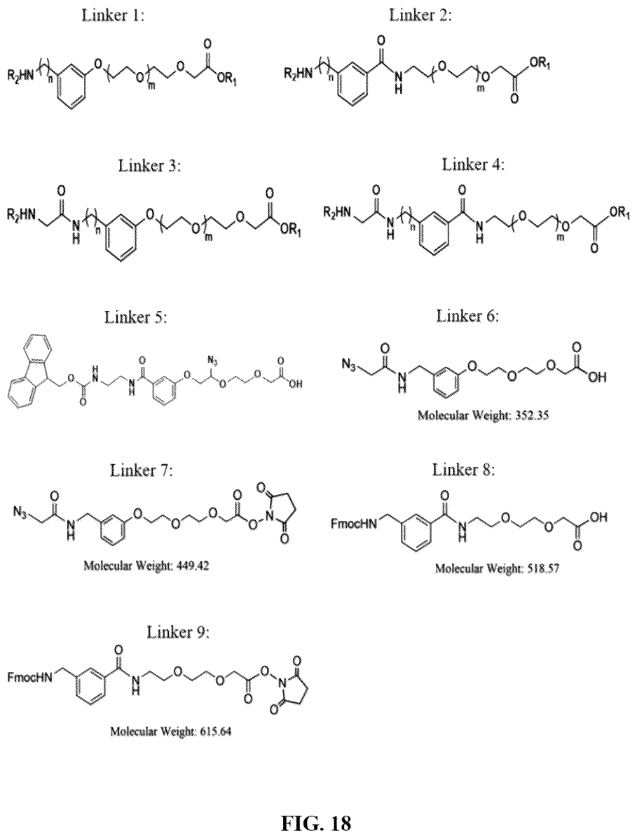

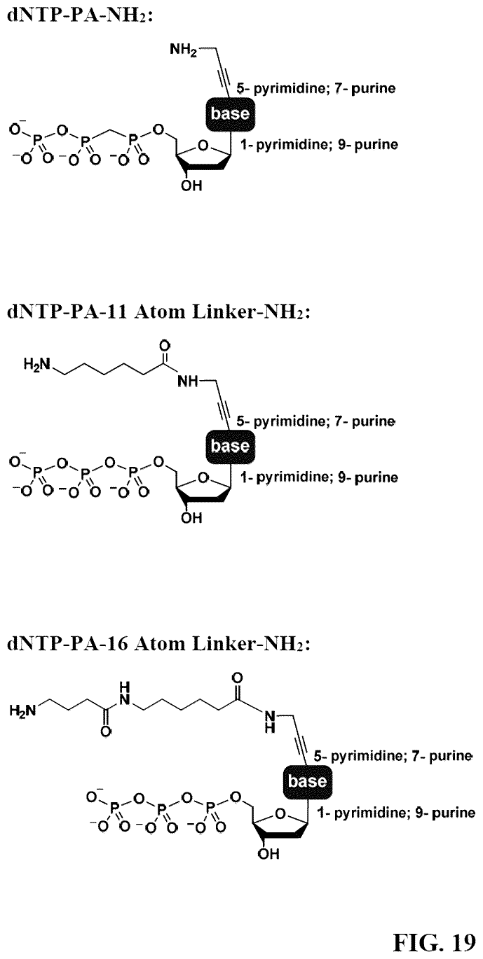

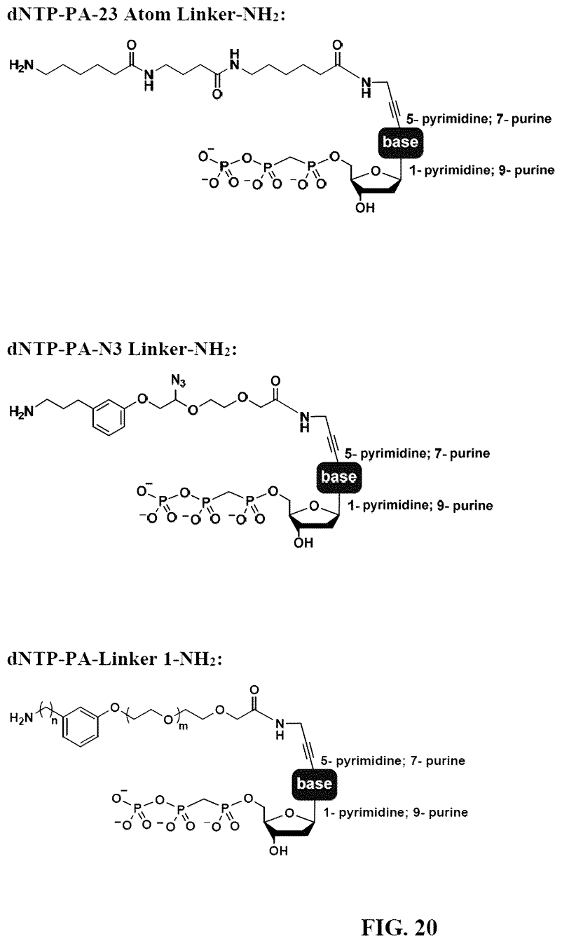

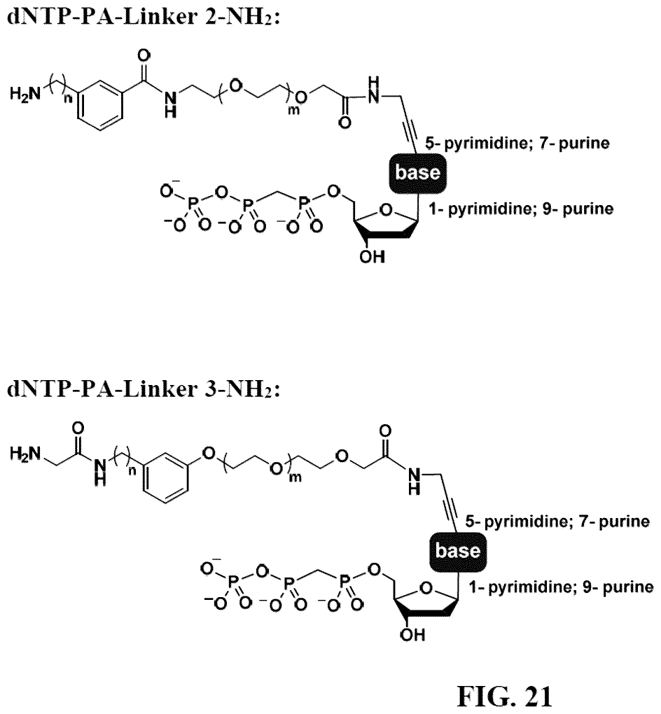

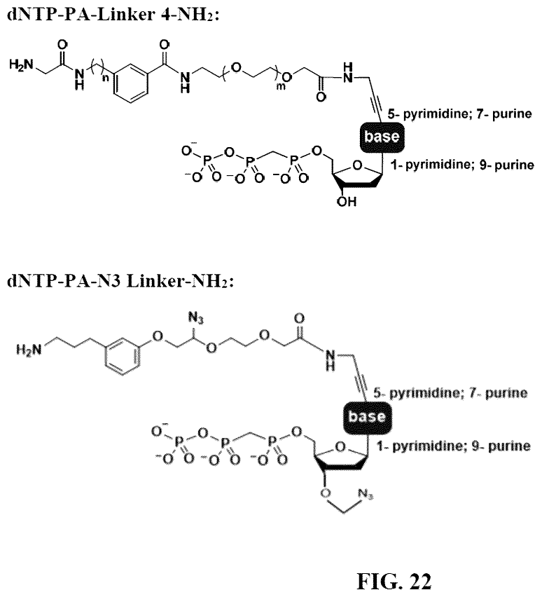

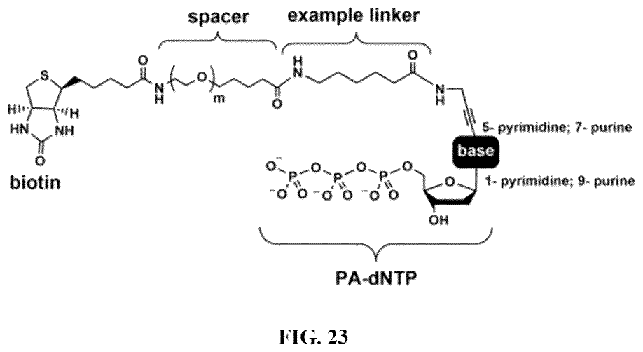

In some embodiments, the multivalent molecule comprises a plurality of nucleotide arms attached to a core and having any configuration including a starburst, helter skelter, or bottle brush configuration (e.g., FIG. 12). The multivalent molecule comprises: (1) a core; and (2) a plurality of nucleotide arms which comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, and wherein the linker is attached to the nucleotide unit. In some embodiments, the nucleotide unit comprises a base, sugar and at least one phosphate group, and the linker is attached to the nucleotide unit through the base. In some embodiments, the linker comprises an aliphatic chain or an oligo ethylene glycol chain where both linker chains having 2-6 subunits. In some embodiments, the linker also includes an aromatic moiety. An example of a nucleotide arm is shown in FIG. 16. Examples of multivalent molecules are shown in FIGS. 12-15. An example of a spacer is shown in FIG. 17 (top) and examples of linkers are shown in FIG. 17 (bottom) and FIG. 18. Examples of nucleotides attached to a linker are shown in FIGS. 19-22. An example of a biotinylated nucleotide arm is shown in FIG. 23.

In some embodiments, a multivalent molecule comprises a core attached to multiple nucleotide arms, and wherein the multiple nucleotide arms have the same type of nucleotide unit which is selected from the group consisting of dATP, dGTP, dCTP, dTTP and dUTP.

In some embodiments, a multivalent molecule comprises a core attached to multiple nucleotide arms, where each arm includes a nucleotide unit. The nucleotide unit comprises an aromatic base, a five carbon sugar (e.g., ribose or deoxyribose), and one or more phosphate groups (e.g., 1-10 phosphate groups). The plurality of multivalent molecules can comprise one type of multivalent molecule having one type of nucleotide unit selected from the group consisting of dATP, dGTP, dCTP, dTTP and dUTP. The plurality of multivalent molecules can comprise at a mixture of any combination of two or more types of multivalent molecules, where individual multivalent molecules in the mixture comprise nucleotide units selected from the group consisting of dATP, dGTP, dCTP, dTTP, dUTP, or a combination thereof.

In some embodiments, the nucleotide unit comprises a chain of one, two or three phosphorus atoms where the chain is attached to the 5′ carbon of the sugar moiety via an ester or phosphoramide linkage. In some embodiments, at least one nucleotide unit is a nucleotide analog having a phosphorus chain in which the phosphorus atoms are linked together with intervening O, S, NH, methylene, or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, the multivalent molecule comprises a core attached to multiple nucleotide arms, and wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to the 3′ sugar position where the sugar comprises a ribose or deoxyribose sugar moiety. In some embodiments, the chain terminating moiety is removable/cleavable from the 3′ sugar position to generate a nucleotide having a 3′OH sugar group which is extendable with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide unit, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh3)4) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K2 CO3) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, the nucleotide unit comprises a chain terminating moiety (e.g., blocking moiety) at the sugar 2′ position, at the sugar 3′ position, or at the sugar 2′ and 3′ positions. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, the nucleotide unit comprising a chain terminating moiety which is selected from the group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, and derivatives thereof.

In some embodiments, the multivalent molecule comprises a core attached to multiple nucleotide arms, wherein the nucleotide arms comprise a spacer, linker and nucleotide unit, and wherein the core, linker and/or nucleotide unit is labeled with detectable reporter moiety. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, at least one nucleotide arm of a multivalent molecule has a nucleotide unit that is attached to a detectable reporter moiety. In some embodiments, the detectable reporter moiety is attached to the nucleotide base. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, the core of a multivalent molecule comprises an avidin-like or streptavidin-like moiety and the core attachment moiety comprises biotin. In some embodiments, the core comprises a streptavidin-type or avidin-type moiety which includes an avidin protein, as well as any derivatives, analogs and other non-native forms of avidin that can bind to at least one biotin moiety. Other forms of avidin moieties include native and recombinant avidin and streptavidin as well as derivatized molecules, e.g. non-glycosylated avidin and truncated streptavidins. For example, avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN, CAPTAVIDIN, NEUTRAVIDIN and NEUTRALITE AVIDIN.

In some embodiments, any of the methods for sequencing nucleic acid molecules described herein can include forming a binding complex, where the binding complex comprises (i) a polymerase, a nucleic acid template molecule duplexed with a primer, and a nucleotide, or the binding complex comprises (ii) a polymerase, a nucleic acid template molecule duplexed with a primer, and a nucleotide unit of a multivalent molecule. In some embodiments, the binding complex has a persistence time of greater than about 0.1, 0.2, 0.3, 0.4, 0.5, 0.6, 0.7, 0.8, 0.9 or 1 second. The binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds, and/or wherein the method is or may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water. In some embodiments, the present disclosure provides said method wherein the binding complex is deposited on, attached to, or hybridized to, a surface showing a contrast-to-noise ratio in the detecting step of greater than 20. In some embodiments, the present disclosure provides said method wherein the contacting is performed under a condition that stabilizes the binding complex when the nucleotide or nucleotide unit is complementary to a next base of the template nucleic acid, and destabilizes the binding complex when the nucleotide or nucleotide unit is not complementary to the next base of the template nucleic acid.

### Methods for Sequencing Using Phosphate Chain-Labeled Nucleotides

In some embodiments, the methods herein can be used for autofocusing of optical systems that can be used for sequencing using immobilized sequencing polymerases which bind non-immobilized template molecules. The present disclosure provides methods for sequencing using immobilized sequencing polymerases which bind non-immobilized template molecules, wherein the sequencing reactions are conducted with phosphate-chain labeled nucleotides. In some embodiments, the sequencing methods comprise an operation (a): providing a support having a plurality of sequencing polymerases immobilized thereon. In some embodiments, the sequencing polymerase comprises a processive DNA polymerase. In some embodiments, the sequencing polymerase comprises a wild type or mutant DNA polymerase, including, for example, a Phi29 DNA polymerase. In some embodiments, the support comprise a plurality of separate compartments and a sequencing polymerase is immobilized to the bottom of a compartment. In some embodiments, the separate compartments comprise a silica bottom through which light can penetrate. In some embodiments, the separate compartments comprise a silica bottom configured with a nanophotonic confinement structure comprising a hole in a metal cladding film (e.g., aluminum cladding film). In some embodiments, the hole in the metal cladding has a small aperture, for example, approximately 70 nm. In some embodiments, the height of the nanophotonic confinement structure is approximately 100 nm. In some embodiments, the nanophotonic confinement structure comprises a zero mode waveguide (ZMW). In some embodiments, the nanophotonic confinement structure contains a liquid.

In some embodiments, the sequencing method further comprises an operation (b): contacting the plurality of immobilized sequencing polymerases with a plurality of single stranded circular nucleic acid template molecules and a plurality of oligonucleotide sequencing primers, under a condition suitable for individual immobilized sequencing polymerases to bind a single stranded circular template molecule, and suitable for individual sequencing primers to hybridize to individual single stranded circular template molecules, thereby generating a plurality of polymerase/template/primer complexes. In some embodiments, the individual sequencing primers hybridize to a universal sequencing primer binding site on the single stranded circular template molecule.

In some embodiments, the sequencing method further comprises an operation (c): contacting the plurality of polymerase/template/primer complexes with a plurality of phosphate chain labeled nucleotides each comprising an aromatic base, a five carbon sugar (e.g., ribose or deoxyribose), and a phosphate chain comprising 3-20 phosphate groups, where the terminal phosphate group is linked to a detectable reporter moiety (e.g., a fluorophore). The first, second and third phosphate groups can be referred to as alpha, beta and gamma phosphate groups. In some embodiments, a particular detectable reporter moiety which is attached to the terminal phosphate group corresponds to the nucleotide base (e.g., dATP, dGTP, dCTP, dTTP or dUTP) to permit detection and identification of the nucleo-base. In some embodiments, the plurality of polymerase/template/primer complexes are contacted with the plurality of phosphate chain labeled nucleotides under a condition suitable for polymerase-catalyzed nucleotide incorporation. In some embodiments, the sequencing polymerases are capable of binding a complementary phosphate chain labeled nucleotide and incorporating the complementary nucleotide opposite a nucleotide in a template molecule. In some embodiments, the polymerase-catalyzed nucleotide incorporation reaction cleaves between the alpha and beta phosphate groups thereby releasing a multi-phosphate chain linked to a fluorophore.

In some embodiments, the sequencing method further comprises an operation (d): detecting the fluorescent signal emitted by the phosphate chain labeled nucleotide that is bound by the sequencing polymerase and incorporated into the terminal end of the sequencing primer. In some embodiments, the operation (d) further comprises identifying the phosphate chain labeled nucleotide that is bound by the sequencing polymerase and incorporated into the terminal end of the sequencing primer.

In some embodiments, the sequencing method further comprises an operation (e): repeating steps (c)-(d) at least once. In some embodiments, sequencing methods that employ phosphate chain labeled nucleotides can be conducted according to the methods described in U.S. Pat. Nos. 7,170,050; 7,302,146; and/or 7,405,281, each of which is incorporated by reference in its entirety.

The headings provided herein are not limitations of the various aspects of the disclosure, which aspects can be understood by reference to the specification as a whole.

### Certain Terminology

Unless defined otherwise, technical and scientific terms used herein have meanings that are commonly understood by those of ordinary skill in the art unless defined otherwise. Generally, terminologies pertaining to techniques of molecular biology, nucleic acid chemistry, protein chemistry, genetics, microbiology, transgenic cell production, and hybridization described herein are those well-known and commonly used in the art. Techniques and procedures described herein are generally performed according to conventional methods well-known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000). See also Ausubel et al., Current Protocols in Molecular Biology, Greene Publishing Associates (1992). The nomenclatures utilized in connection with, and the laboratory procedures and techniques described herein are those well-known and commonly used in the art.

Unless otherwise required by context herein, singular terms shall include pluralities and plural terms shall include the singular. Singular forms “a” “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include: “A and B”; “A or B”; “A” (A alone); and “B” (B alone). In a similar manner, the term “and/or” as used in a phrase such as “A, B, and/or C” is intended to encompass each of the following aspects: “A, B, and C”; “A, B, or C”; “A or C”; “A or B”; “B or C”; “A and B”; “B and C”; “A and C”; “A” (A alone); “B” (B alone); and “C” (C alone).

As used herein and in the appended claims, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the terms “about,” “approximately,” and “substantially” refer to a value or composition that is within an acceptable error range for the particular value or composition as determined by one of ordinary skill in the art, which will depend in part on how the value or composition is measured or determined, i.e., the limitations of the measurement system. For example, “about,” “approximately,” or “substantially” can mean within one or more than one standard deviation per the practice in the art. Alternatively, “about” or “approximately” can mean a range of up to 10% (i.e., ±10%) or more depending on the limitations of the measurement system.

For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about,” “approximately,” “substantially” should be assumed to be within an acceptable error range for that particular value or composition. Also, where ranges and/or subranges of values are provided, the ranges and/or subranges can include the endpoints of the ranges and/or subranges.

The term “polony” used herein refers to a nucleic acid library molecule that can be clonally amplified in-solution or on-support to generate an amplicon that can serve as a template molecule for sequencing. In some embodiments, a linear library molecule can be circularized to generate a circularized library molecule, and the circularized library molecule can be clonally amplified in-solution or on-support to generate a concatemer. In some embodiments, the concatemer can serve as a nucleic acid template molecule which can be sequenced. The concatemer is sometimes referred to as a polony. In some embodiments, a polony includes nucleotide strands.

References herein to “one embodiment,” “an embodiment,” “an example embodiment,” “some embodiments,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein.

It is to be appreciated that the Detailed Description section, and not any other section, is intended to be used to interpret the claims. Other sections may set forth one or more but not all examples of embodiments as contemplated by the inventor(s), and thus, are not intended to limit this disclosure or the appended claims in any way.

While this disclosure describes examples of embodiments for some fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries may be defined as long as the specified functions and relationships (or equivalents thereof) are appropriately performed. Also, alternative embodiments may perform functional blocks, steps, operations, methods, etc. using orderings different from those described herein.

### EXAMPLES

These examples are provided for illustrative purposes only and not to limit the scope of the claims provided herein.

### Example 1—Optical System Efficiency

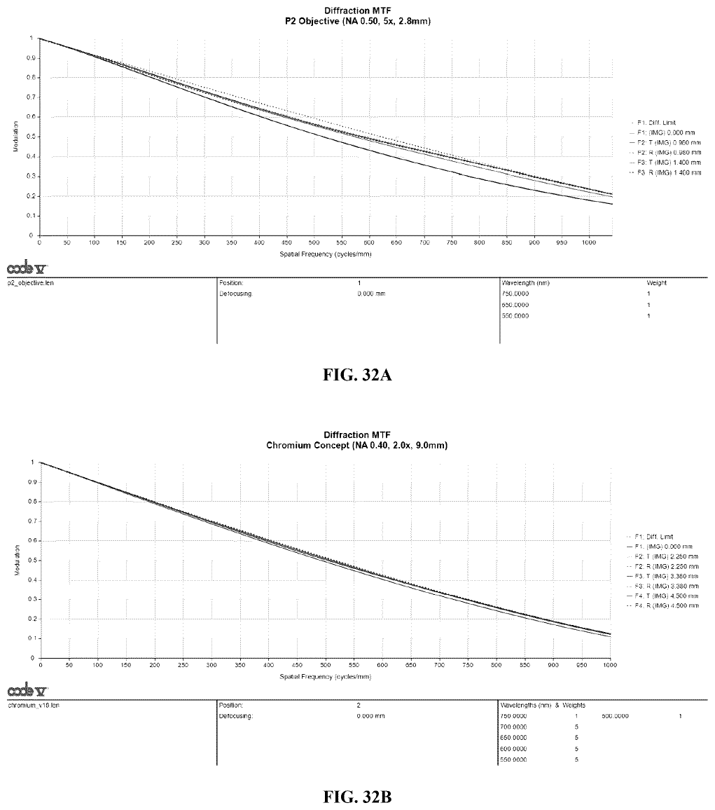

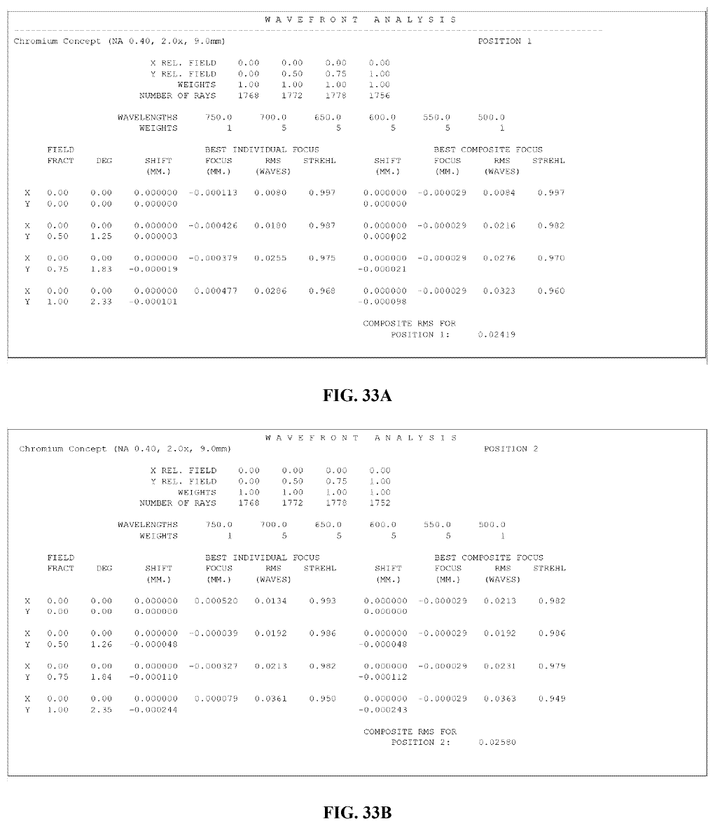

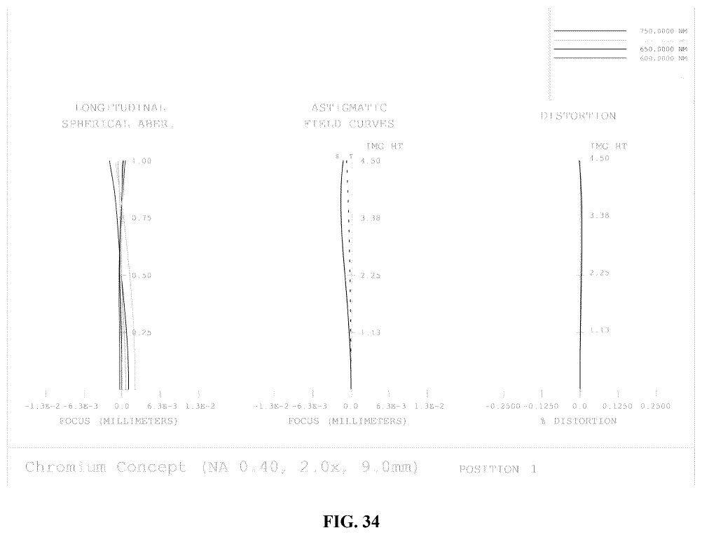

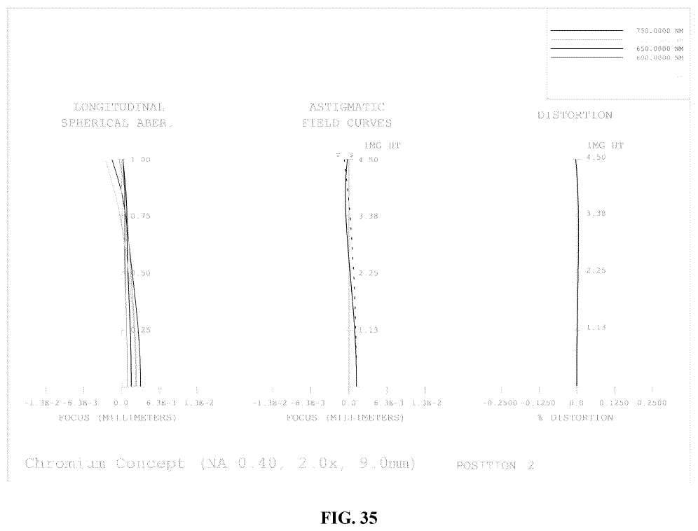

FIGS. 32A-32B provide diffraction modulation transfer functions (MTFs) for optical systems, according to some embodiments. FIG. 32A shows the MTF of an objective based system with a p=480 nm, while FIG. 32B shows the MTF of an optical system of the present disclosure with a p=500 nm. As can be seen, the overall effectiveness of the optical system of the present disclosure is higher at all spatial frequencies than the objective based system, and the system of the present disclosure provides a much larger field of view (9 mm versus the 2 mm of the objective based system). FIGS. 33A-33B show wavefront analysis calculations for an optical system of the present disclosure, according to some embodiments. In both cases, over a wide field of view (e.g., 9 mm), the optical systems demonstrated low composite root-mean-square errors of about 24 mλ and 26 mλ, respectively.

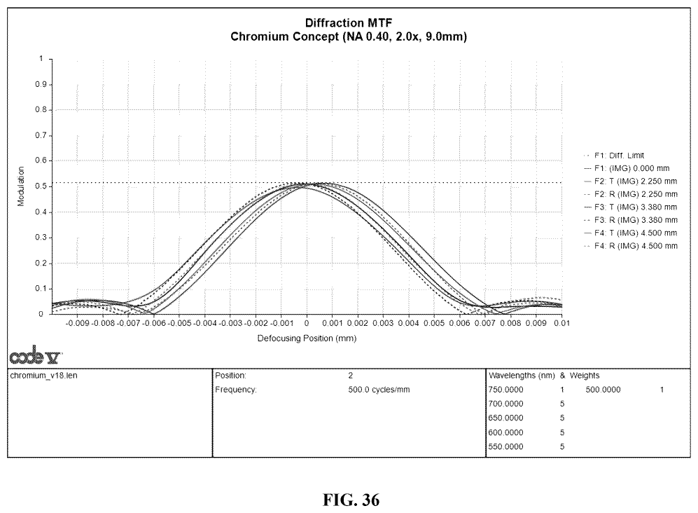

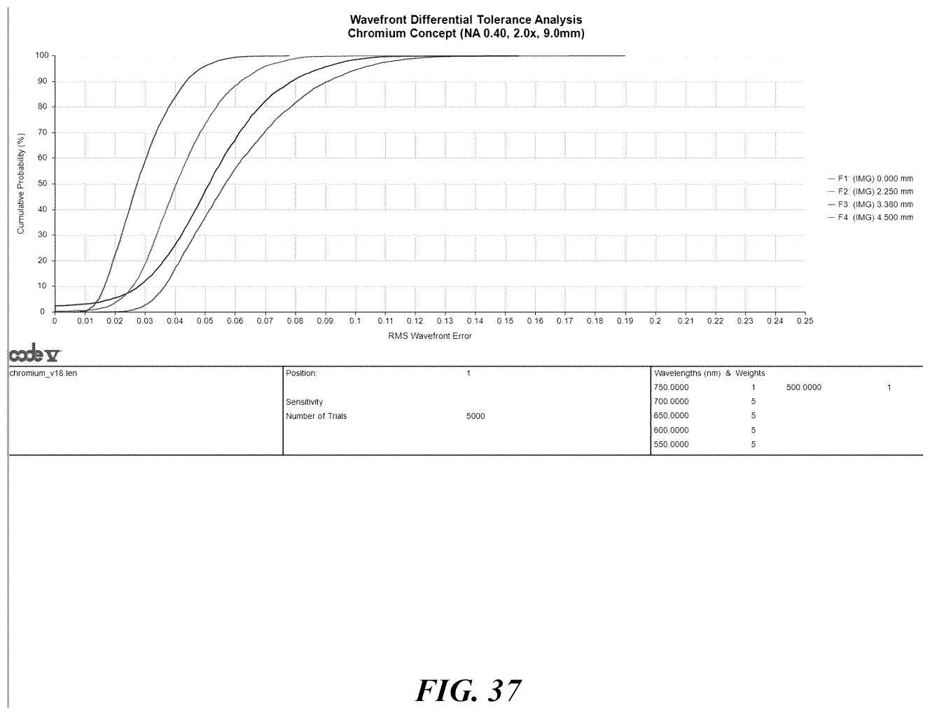

FIGS. 34-35 show a top and bottom, respectively, surface optical performance curve, according to some embodiments. In both cases, the longitudinal spherical aberration, astigmatic field curve, and distortion are low, indicating improved optical (e.g., imaging) performance. FIG. 36 shows a plot of an MTF of an optical system, according to some embodiments. The MTF plot can show a wide depth of field (e.g., ±1 micrometer) with low field curvature, which can show the ability of the optical system to provide wide area, large depth of field imaging capable of imaging many samples on a solid support at a same time. In some cases, the wide depth of field can enable multi-surface imaging (e.g., imaging a plurality of surfaces of a solid support at a same time). FIG. 37 shows a plot of a cumulative probability of achieving a given wavefront error, according to some embodiments. The plot shows data regarding the low wavefront errors that the systems of the present disclosure can achieve.

### NUMBERED EMBODIMENTS OF THE DISCLOSURE

1. An optical system, comprising:
   a stage configured to hold a solid support;
   a light source configured to illuminate said solid support; and
   an optical assembly disposed at least partly within an optical path from said stage to said light source, wherein said optical assembly is configured to provide an illumination over an area of said solid support that is greater than about 20 square millimeters (mm²) with a peak-to-valley variation of at most about 5%.
2. The optical system of any one of the preceding embodiments, wherein said optical assembly does not comprise an objective.
3. The optical system of any one of the preceding embodiments, wherein said optical system does not comprise said objective.
4. The optical system of any one of the preceding embodiments, wherein said optical assembly does not comprise a tube lens.
5. The optical system of any one of the preceding embodiments, wherein said optical system does not comprise said tube lens.
6. The optical system of any one of the preceding embodiments, wherein said stage does not adjust in an optical axis of said system.
7. The optical system of any one of the preceding embodiments, wherein said illumination has an irradiance of at least about 40 milliwatts per square millimeter.
8. The optical system of any one of the preceding embodiments, wherein said optical assembly is configured to receive an emission light from said solid support.
9. The optical system of any one of the preceding embodiments, wherein said optical assembly has a numerical aperture (NA) of at least about 0.3.
10. The optical system of any one of the preceding embodiments, wherein said emission light has a wavelength of about 500 nanometers to about 750 nanometers.
11. The optical system of any one of the preceding embodiments, wherein said optical assembly has a working distance of at least about 1 mm to 25 mm.
12. The optical system of any one of the preceding embodiments, further comprising a motion coil housed within said optical assembly configured to move a focusing element within said optical path of said optical system.
13. The optical system of any one of the preceding embodiments, wherein a motor external to the optical system is configured to move a focusing element along the optical axis in one or both directions.
14. The optical system of any one of the preceding embodiments, wherein said motor is coupled directly with a piece of a first, second, or third housing of the optical assembly, and the piece of the first, second, or third housing of the optical assembly is coupled directly with the focusing element.
15. The optical system of any one of the preceding embodiments, wherein said light source is a pulsed light source.
16. The optical system of any one of the preceding embodiments, wherein said optical system has a composite root mean square error of less than about 0.05.
17. The optical system of any one of the preceding embodiments, wherein said optical assembly has an illumination efficiency of at least about 90%.
18. The optical system of any one of the preceding embodiments, wherein said area is greater than 30 mm².
19. The optical system of any one of the preceding embodiments, wherein said area is greater than 50 mm² or 60 mm².
20. The optical system of any one of the preceding embodiments, further comprising said solid support within said stage.
21. The optical system of any one of the preceding embodiments, wherein said solid support comprises two or more surfaces having one or more samples immobilized thereon which are imaged by said optical system.
22. The optical system of any one of the preceding embodiments, wherein said solid support comprises three or more surfaces having one or more samples immobilized thereon imaged by said optical system.
23. The optical system of any one of the preceding embodiments, wherein said three or more surfaces are axially displaced from each other at least along an optical axis of the optical system.
24. The optical system of any one of the preceding embodiments, wherein said solid support comprises a probe configured to bind a nucleic acid molecule.
25. The optical system of any one of the preceding embodiments, wherein said probe is bound to a surface of said solid support.
26. The optical system of any one of the preceding embodiments, wherein said light source is a laser light source.
27. The optical system of any one of the preceding embodiments, wherein said optical assembly comprises a dichroic filter configured to transmit said illumination.
28. The optical system of any one of the preceding embodiments, wherein said optical assembly comprises a first segment comprising a first housing comprising a first plurality of lenses, a second segment comprising a second housing, and a third segment comprising a third housing comprising a second plurality of lenses.
29. The optical system of any one of the preceding embodiments, wherein said first segment and said third segment are optically aligned.
30. The optical system of any one of the preceding embodiments, wherein said first segment is positioned between said third segment and said stage.
31. The optical system of any one of the preceding embodiments, wherein said third segment is positioned between said first segment and an image sensor of the optical system.
32. The optical system of any one of the preceding embodiments, wherein said first plurality of lenses are movable along said optical path with a range of about 0 to about 2 millimeters.
33. The optical system of any one of the preceding embodiments, wherein said first plurality of lenses comprises an asymmetric convex-convex lens.
34. The optical system of any one of the preceding embodiments, wherein said second plurality of lenses comprises an asymmetric concave-concave lens.
35. The optical system of any one of the preceding embodiments, wherein said asymmetric concave-concave lens is an aspheric asymmetric concave-concave lens.
36. The optical system of any one of the preceding embodiments, wherein said optical system is configured to acquire images of the solid support without moving an optical compensator into the optical path between the solid support and a detector of the optical system.
37. The optical system of any one of the preceding embodiments, wherein said optical system is configured to acquire images of the solid support without moving an optical compensator out from the optical path between the sample and a detector of the optical system.
38. The optical system of any one of the preceding embodiments, wherein said solid support is a flow cell.
39. The optical system of any one of the preceding embodiments, wherein said optical assembly is configured to generate one or more spatial constrictions lateral to said optical path of light which travels therethrough.
40. The optical system of any one of the preceding embodiments, wherein said optical assembly is configured to generate one or more field curvature corrections lateral to said optical path of light which travels therethrough.
41. The optical system of any one of the preceding embodiments, wherein said optical assembly is configured to generate at least one field curvature correction lateral to the optical path of light travels therethrough in a first segment, second segment, or third segment.
42. A method of analyzing a biological molecule, comprising:
   (a) providing a solid support comprising said biological molecule comprising a label;
   (b) using an optical system comprising a light source to provide illumination to said biological molecule comprising said label, thereby generating a signal light or a change thereof, wherein said illumination is provided over an area of said solid support that is greater than about 20 square millimeters (mm²) with a peak-to-valley variation of at most about 5%;
   (c) detecting, using a detector of said optical system, said signal light or said change thereof; and
   (d) processing at least in part said signal light or said change thereof to analyze said biological molecule.
43. The method of any one of the preceding embodiments, wherein said biological molecule is a nucleic acid molecule, a protein, or a polypeptide.
44. The method of any one of the preceding embodiments, wherein said biological molecule is a nucleic acid.
45. The method of any one of the preceding embodiments, further comprising, prior to (a), binding said biological molecule to a probe bound to said solid support, and coupling said label to said biological molecule.
46. The method of any one of the preceding embodiments, wherein said label is coupled to said biological molecule by hybridization.
47. The method of any one of the preceding embodiments, wherein said optical system does not comprise an objective.
48. The method of any one of the preceding embodiments, wherein said solid support is not moved in an optical axis of said optical system.
49. The method of any one of the preceding embodiments, wherein a plurality of images of said solid support are acquired without moving said solid support in said optical axis.
50. The method of any one of the preceding embodiments, wherein said illumination has an irradiance of at least about 40 milliwatts per square millimeter.
51. The method of any one of the preceding embodiments, wherein said signal light has a wavelength of about 500 nanometers to about 750 nanometers.
52. The method of any one of the preceding embodiments, wherein said detecting of (c) is performed using an optical element with a numerical aperture of at least about 0.3.
53. The method of any one of the preceding embodiments, further comprising, in (b), using a motion coil within said optical system to move a focusing element within an optical path of said optical system, thereby changing a focus of said optical system on said solid support.
54. The method of any one of the preceding embodiments, wherein said light source is a pulsed light source.
55. The method of any one of the preceding embodiments, wherein said illumination is provided with an efficiency of at least about 90%.
56. The method of any one of the preceding embodiments, further comprising repeating (b)-(d) for an additional biological molecule coupled to an additional surface of said solid support.
57. The method of any one of the preceding embodiments, further comprising, subsequent to (c), removing said label from said biological molecule.
58. The method of any one of the preceding embodiments, further comprising repeating (a)-(d) for an additional label that binds to another portion of the biological molecule.
59. The method of any one of the preceding embodiments, wherein said optical assembly is configured to generate one or more spatial constrictions lateral to said optical path of light that travels therethrough.
60. The method of any one of the preceding embodiments, wherein said optical assembly is configured to generate one or more field curvature corrections lateral to said optical path of light that travels therethrough.
61. The method of any one of the preceding embodiments, wherein said optical assembly is configured to generate at least one field curvature correction lateral to the optical path of light that travels therethrough in a first segment, second segment, or third segment.
62. The method of any one of the preceding embodiments, wherein (d) comprises processing at least in part said signal light or said change thereof to generate one or more solid support images and analyze said one more solid support images to generate base calls of the sample.
63. The method of any one of the preceding embodiments, wherein each of said solid support images comprises a field-of-view (FOV) that is greater than 20 square millimeters (mm²).
64. The method of any one of the preceding embodiments, wherein said solid support is a flow cell.
65. An optical system, comprising:
   a stage configured to hold a solid support;
   a light source configured to illuminate said solid support; and
   a despeckler optically coupled to said light source and disposed within an optical path from said light source to said stage.
66. The optical system of any one of the preceding embodiments, further comprising an additional light source optically coupled into said despeckler.
67. The optical system of any one of the preceding embodiments, wherein light from said additional light source is configured to illuminate said solid support with a different wavelength of light from said light source.
68. The optical system of any one of the preceding embodiments, wherein at least about 4 light sources are coupled into said despeckler.
69. The optical system of any one of the preceding embodiments, wherein said despeckler is a vibrational despeckler.
70. The optical system of any one of the preceding embodiments, wherein said despeckler is a passive despeckler.
71. The optical system of any one of the preceding embodiments, wherein said passive despeckler comprises a diffuse scattering plate.
72. The optical system of any one of the preceding embodiments, wherein said despeckler is a tension despeckler.
73. The optical system of any one of the preceding embodiments, wherein said despeckler is configured to reduce speckle noise to at most about 5%.
74. The optical system of any one of the preceding embodiments, wherein said solid support is a flow cell.
75. A method for analyzing a biological molecule, comprising:
   (a) providing a solid support comprising a biological sample comprising a label;
   (b) using an optical system comprising a light source to provide illumination to said biological sample comprising said label, thereby generating a signal light or a change thereof, wherein said illumination is provided through a despeckler in an optical path of said optical system;
   (c) detecting, using a detector of said optical system, said signal light or said change thereof; and
   (d) processing at least in part said signal light or said change thereof to analyze said biological molecule.
76. The method of any one of the preceding embodiments, further comprising repeating (b)-(d) for an additional biological sample coupled to an additional surface of said solid support.
77. The method of any one of the preceding embodiments, further comprising, subsequent to (c), removing said label from said biological sample.
78. The method of any one of the preceding embodiments, further comprising repeating (a)-(d) for an additional label that binds to the biological sample.
79. The method of any one of the preceding embodiments, wherein said despeckler uses vibration to despeckle said illumination.
80. The method of any one of the preceding embodiments, further comprising using an additional light source to illuminate said solid support.
81. The method of any one of the preceding embodiments, wherein said additional light source provides a different wavelength of light to said solid support.
82. The method of any one of the preceding embodiments, wherein said additional light source is optically coupled to said despeckler.
83. The method of any one of the preceding embodiments, wherein said biological sample comprises a nucleic acid molecule, a protein, or a polypeptide.
84. The method of any one of the preceding embodiments, wherein said biological sample comprises a nucleic acid.
85. An illumination system for a multi-channel fluorescence imaging module, comprising:
   an illumination subsystem, comprising:
      a light source;
      a despeckler; and
   a light beam delivery subsystem optically coupled to the illumination system, comprising:
      a collimator; and
      one or more optical lens elements.
86. The illumination system of any one of the preceding embodiments, wherein said illumination system is configured to provide an illumination field that is no less than 10 mm², 20 mm², 30 mm², 40 mm², or 50 mm² at a sample plane.
87. The illumination system of any one of the preceding embodiments, wherein said illumination system is configured to provide a power density that is no less than 40, 50, or 60 milliwatts/mm² at a sample plane.
88. The illumination system of any one of the preceding embodiments, wherein said sample plane is orthogonal to a z-axis.
89. The illumination system of any one of the preceding embodiments, wherein said light source comprises one or more lasers.
90. The illumination system of any one of the preceding embodiments, wherein said one or more lasers comprises one or more laser diodes.
91. The illumination system of any one of the preceding embodiments, wherein said one or more lasers emit light of multiple wavelengths.
92. The illumination system of any one of the preceding embodiments, wherein said illumination subsystem further comprises one or more optical fibers.
93. The illumination system of any one of the preceding embodiments, wherein at least one of said optical fibers comprises a length of 0.5 m to 5 m.
94. The illumination system of any one of the preceding embodiments, wherein at least one of said optical fibers comprises a core with a maximum dimension of 50 μm to 1500 μm in a cross-section of the core.
95. The illumination system of any one of the preceding embodiments, wherein said cross-section of the core is circular.
96. The illumination system of any one of the preceding embodiments, wherein the power efficiency of the illumination system is no less than 65%, 70%, 75%, or 80%.
97. The illumination system of any one of the preceding embodiments, wherein the illumination field is of a rectangular or square shape.
98. The illumination system of any one of the preceding embodiments, wherein said one or more optical lens elements comprise one or more multi-lens arrays.
99. The illumination system of any one of the preceding embodiments, wherein each of said one or more multi-lens arrays comprises one or more of: an asymmetric convex-convex lens, a convex-plano lens, a concave-plano lens, an asymmetric concave-concave lens, and an asymmetric convex-concave lens.
100. The illumination system of any one of the preceding embodiments, wherein each of said one or more multi-lens arrays comprises multiple lens elements at least in a direction that is orthogonal to a z-axis.
101. The illumination system of any one of the preceding embodiments, wherein said illumination subsystem further comprises an optical fiber that is optically coupled to the laser diode and mitigates speckle of the light source.
102. The illumination system of any one of the preceding embodiments, wherein said despeckler comprises an optical fiber that is optically coupled to the laser diode and mitigates speckle of the light source.
103. The illumination system of any one of the preceding embodiments, wherein said one or more optical lens elements comprises: an asymmetric convex-convex lens, a convex-plano lens, a concave-plano lens, an asymmetric concave-concave lens, and an asymmetric convex-concave lens, or a combination thereof.
104. The illumination system of any one of the preceding embodiments, wherein said one or more optical lens elements comprises: a first multi-lens array and a second multi-lens array that are positioned along a z-axis between the collimator and an entrance pupil of the illumination system.
105. The illumination system of any one of the preceding embodiments, wherein said illumination system is configured to generate an illumination field at a sample stage that is greater than 50 mm² with less than ±2%, 5%, 8%, 10%, or 12% variance in illumination power density across the illumination field.
106. The illumination system of any one of the preceding embodiments, wherein said despeckler comprises a mechanical vibration source.
107. The illumination system of any one of the preceding embodiments, wherein said despeckler comprises a vibration source that produces vibration at a predetermined frequency range.
108. The illumination system of any one of the preceding embodiments, wherein said mechanical vibration source is configured to vibrate at one or more frequencies in an audible sound range, a ultrasound range, or both.
109. The illumination system of any one of the preceding embodiments, wherein said mechanical vibration source is configured to generate vibrating motions in one, two, or three dimensions.
110. The illumination system of any one of the preceding embodiments, wherein at least a portion of each of said optical fibers or single optical fiber is wound or coiled for one or more rounds.
111. The illumination system of any one of the preceding embodiments, wherein at least a portion of each of said optical fibers or single optical fiber is fixedly or loosely attached to said mechanical vibration source.
112. The illumination system of any one of the preceding embodiments, wherein said despeckler is physically isolated from the sample stage, an objective lens, and said one or more image sensors so that mechanical motion of the despeckler is independent from the sample stage, the objective lens, and the one or more image sensors.
113. The illumination system of any one of the preceding embodiments, wherein said despeckler is configured to reduce speckle noise to be no more than 4%, 4.5%, 5%, or 5.5%.
114. The illumination system of any one of the preceding embodiments, wherein said light source comprises a multi-color laser array.
115. The illumination system of any one of the preceding embodiments, wherein said multi-color laser array comprises an array of laser diodes that emits laser lights at 2, 3, 4, 5, or 6 wavelengths or in 2, 3, 4, 5, or 6 wavelength ranges.
116. The illumination system of any one of the preceding embodiments, wherein said multi-color laser array comprise lasers that emit light of 2, 3, or 4 color wavelengths or wavelength ranges at least in a direction that is orthogonal to a z-axis.
117. The illumination system of any one of the preceding embodiments, wherein said illumination subsystem further comprises one or more coupling lens.
118. The illumination system of any one of the preceding embodiments, wherein said illumination subsystem comprises a single optical fiber.
119. The illumination system of any one of the preceding embodiments, wherein said single optical fiber comprises a core with a maximum dimension of 500 μm to 1500 μm in a cross-section of the core.
120. The illumination system of any one of the preceding embodiments, wherein a power in the light beam delivery subsystem is greater than 5, 8, 10, 12, or 14 Watts for one or more wavelengths or wavelength ranges.
121. The illumination system of any one of the preceding embodiments, wherein a power at a sample plane is greater than 5, 8, 10, 12, or 14 Watts for one or more wavelengths or wavelength ranges.
122. The illumination system of any one of the preceding embodiments, wherein the illumination subsystem further comprises a plurality of optical fibers, each optical fiber optically coupled to one or more corresponding lasers of the light source, wherein the one or more corresponding lasers emit light of a same wavelength or wavelength range as the light source.
123. The illumination system of any one of the preceding embodiments, wherein said illumination subsystem further comprises one or more dichroic filters.
124. The illumination system of any one of the preceding embodiments, wherein said light source comprises multiple light beam combiners.
125. The illumination system of any one of the preceding embodiments, wherein said light source comprises multiple polarization light beam combiners.
126. The illumination system of any one of the preceding embodiments, wherein said light source comprises two or more lasers that emit light at a same wavelength or in a same wavelength range.
127. The illumination system of any one of the preceding embodiments, wherein each ofs said polarization light beam combiners is configured to combine light emitted from two or more lasers at the same light wavelength or in the same light wavelength range.
128. The illumination system of any one of the preceding embodiments, wherein said despeckler is positioned in an optical path between a collimator and the sample plane.
129. The illumination system of any one of the preceding embodiments, wherein said despeckler is positioned where a diameter of an optical beam is greater than 5 mm, 10 mm, or 20 mm, and wherein the diameter of the optical beam is orthogonal to a z-axis.
130. The illumination system of any one of the preceding embodiments, wherein said optical fiber comprises a core with a rectangular or square cross-section.
131. The illumination system of any one of the preceding embodiments, wherein said optical fiber comprises a core with a non-circular cross-section.
132. The illumination system of any one of the preceding embodiments, wherein said illumination subsystem further comprises one or more liquid light guides.
133. The illumination system of any one of the preceding embodiments, wherein said one or more liquid light guides are optically coupled to the light source in the absence of an optical fiber.
134. The illumination system of any one of the preceding embodiments, wherein said one or more liquid light guides comprise a liquid core with a maximum dimension of 0.5 mm to 10 mm in a cross-section of the liquid core, and wherein the cross-section is orthogonal to a z-axis.
135. The illumination system of any one of the preceding embodiments, wherein said liquid core comprises a cross-section that is circular.
136. The illumination system of any one of the preceding embodiments, wherein said liquid core comprises a cross-section that is non-circular.
137. An imaging module for multi-channel fluorescence imaging, comprising:
   the illumination system in any one of the preceding embodiments; and
   an image acquisition system configured to acquire flow cell images of a sample immobilized on a sample stage at a sample plane.
138. The imaging module of any one of the preceding embodiments, wherein each of said flow cell images comprises a field-of-view (FOV) that is greater than 20 mm², 30 mm², 40 mm², or 50 mm².
139. The imaging module of any one of the preceding embodiments, wherein each of said flow cell images comprises a field-of-view (FOV) that overlaps with an illumination field generated by the illumination system at the sample plane.
140. The imaging module of any one of the preceding embodiments, wherein each of said flow cell images comprises a field-of-view (FOV) that overlaps with at least 80%, 85%, 90%, 95% of an illumination field generated by the illumination system at the sample plane.
141. The imaging module of any one of the preceding embodiments, wherein each of said flow cell images comprises a field-of-view (FOV) with a size that is at least 80%, 85%, 90%, 95% of an illumination field generated by the illumination system at the sample plane.
142. The imaging module of any one of the preceding embodiments, wherein said imaging module comprises:
   one or more image sensors; and
   an objective lens.
143. The imaging module of any one of the preceding embodiments, wherein the numerical aperture (NA) of the imaging module is greater than 0.4, 0.5, or 0.6.
144. The imaging module of any one of the preceding embodiments, wherein said imaging module comprise an optical axis that is parallel with a z-axis.
145. The imaging module of any one of the preceding embodiments, wherein said flow cell images are of the sample immobilized on one or more surfaces of a solid support.
146. The imaging module of any one of the preceding embodiments, wherein said one or more surfaces comprises 2 or more surfaces that are axially displaced from each other at least along a z-axis.
147. The imaging module of any one of the preceding embodiments, wherein said flow cell images are acquired without moving an optical compensator into an optical path between the objective lens and the one or more image sensors.
148. The imaging module of any one of the preceding embodiments, wherein said flow cell images of one or more surfaces are acquired without moving an optical compensator out from an optical path between the objective lens and the at least one image sensor.
149. The imaging module of any one of the preceding embodiments, wherein each of said flow cell images comprises a contrast to noise ratio (CNR) of at least 5 when: nucleic acid polonies disposed on the three or more surfaces are labeled with cyanine dye 3 (Cy3); the dichroic mirror and bandpass filter set are optimized for Cy3 emission; and the flow cell image is acquired by the optical system under non-signal saturating conditions while one or more of the surfaces is immersed in 25 mM ACES, pH 7.4 buffer.
150. The imaging module of any one of the preceding embodiments, wherein said imaging module is configured for determining nucleotide in the nucleic acid molecules in the sample.
151. The imaging module of any one of the preceding embodiments, wherein said imaging module is configured for performing sequencing-by-avidity, sequencing-by-nucleotide base-pairing, sequencing-by-nucleotide binding, or sequencing-by-nucleotide incorporation reactions on at least one of said one or more surfaces.
152. The imaging module of any one of the preceding embodiments, wherein each of said one or more surfaces comprises a plurality of primed target nucleic acid sequences coupled thereto, wherein a primed target nucleic acid sequence of the plurality of primed target nucleic acid sequences has a polymerase bound thereto.
153. A method for sequencing nucleic acid molecules comprising:
   providing a flow cell comprising one or more surfaces, wherein each surface comprises:
      at least one hydrophilic polymer coating layer;
      a plurality of oligonucleotide molecules attached to the at least one hydrophilic polymer coating layer; and
      at least one discrete region of each surface that comprises a plurality of clonally-amplified, sample nucleic acid molecules immobilized to the plurality of attached oligonucleotide molecules;
   causing, by an illumination system, the plurality of clonally amplified sample nucleic acid molecules in an illumination field to fluoresce in on and off events in different colors; and
   detecting, by the one or more image sensors, the on and off events in one or more color channels as the on and off events are occurring for the plurality of clonally amplified sample nucleic acid molecules to determine an identity of a nucleotide of the clonally amplified sample nucleic acid molecule.
154. The method of any one of the preceding embodiments, wherein said method further comprises:
   adjusting the NA of an imaging module.
155. The method of any one of the preceding embodiments, wherein said method further comprises:
   changing the NA of an imaging module in a range from 0.4 to 0.6 by changing an adjustable size of an optical aperture.
156. The method of any one of the preceding embodiments, wherein said illumination system is configured to provide an illumination field that is no less than 10 mm², 20 mm², 30 mm², 40 mm², or 50 mm² at a sample plane.
157. The method of any one of the preceding embodiments, wherein said illumination system is configured to provide a power density that is no less than 40, 50, or 60 milliwattsmilliwatts/mm² at a sample plane.
158. The method of any one of the preceding embodiments, wherein said illumination system comprises:
   an illumination subsystem, comprising:
      a light source;
      a despeckler; and
   a light beam delivery subsystem optically coupled to the illumination system, comprising:
      a collimator; and
      one or more optical lens elements.
159. The method of any one of the preceding embodiments, wherein the sample plane is orthogonal to a z-axis.
160. The method of any one of the preceding embodiments, wherein said light source comprises one or more lasers.
161. The method of any one of the preceding embodiments, wherein said one or more lasers comprises one or more laser diodes.
162. The method of any one of the preceding embodiments, wherein said one or more lasers emit light of multiple wavelengths.
163. The method of any one of the preceding embodiments, wherein said illumination subsystem further comprises one or more optical fibers.
164. The method of any one of the preceding embodiments, wherein at least one of said optical fibers comprises a length of 0.5 m to 5 m.
165. The method of any one of the preceding embodiments, wherein at least one of said optical fibers comprises a core with a maximum dimension of 50 μm to 1500 μm in a cross-section of the core.
166. The method of any one of the preceding embodiments, wherein said cross-section of the core is circular.
167. The method of any one of the preceding embodiments, wherein the power efficiency of the illumination system is no less than 65%, 70%, 75%, or 80%.
168. The method of any one of the preceding embodiments, wherein the illumination field is of a rectangular or square shape.
169. The method of any one of the preceding embodiments, wherein said one or more optical lens elements comprise one or more multi-lens arrays.
170. The method of any one of the preceding embodiments, wherein each multi-lens array of said multi-lens arrays comprises one or more of: an asymmetric convex-convex lens, a convex-plano lens, a concave-plano lens, an asymmetric concave-concave lens, and an asymmetric convex-concave lens.
171. The method of any one of the preceding embodiments, wherein each multi-lens array of said multi-lens arrays comprises multiple lens elements at least in a direction that is orthogonal to a z axis.
172. The method of any one of the preceding embodiments, wherein said illumination subsystem further comprises an optical fiber that is optically coupled to the laser diode and mitigates speckle of the light source.
173. The method of any one of the preceding embodiments, wherein said despeckler is comprised of an optical fiber that is optically coupled to the laser diode and mitigates speckle of the light source.
174. The method of any one of the preceding embodiments, wherein said one or more optical lens elements comprise: an asymmetric convex-convex lens, a convex-plano lens, a concave-plano lens, an asymmetric concave-concave lens, and an asymmetric convex-concave lens, or a combination thereof.
175. The method of any one of the preceding embodiments, wherein said one or more optical lens elements comprise: a first multi-lens array and a second multi-lens array that are positioned along a z-axis between the collimator and an entrance pupil of said illumination system.
176. The method of any one of the preceding embodiments, wherein said illumination system is configured to generate an illumination field at the sample stage that is greater than 50 mm² with less than ±2%, 5%, 8%, 10%, or 12% variance in illumination power density across the illumination field.
177. The method of any one of the preceding embodiments, wherein said despeckler comprises a mechanical vibration source.
178. The method of any one of the preceding embodiments, wherein said despeckler comprises a vibration source that produces vibration at a predetermined frequency range.
179. The method of any one of the preceding embodiments, wherein said mechanical vibration source is configured to vibrate at one or more frequencies in an audible sound range, a ultrasound range, or both.
180. The method of any one of the preceding embodiments, wherein said mechanical vibration source is configured to generate vibrating motions in one, two, or three dimensions.
181. The method of any one of the preceding embodiments, wherein at least a portion of each of said optical fibers or single optical fiber is wound or coiled for one or more rounds.
182. The method of any one of the preceding embodiments, wherein at least a portion of each of said optical fibers or single optical fiber is fixedly or loosely attached to the mechanical vibration source.
183. The method of any one of the preceding embodiments, wherein said despeckler is physically isolated from the sample stage, the objective lens, and the one or more image sensors so that mechanical motion of the despeckler is independent from the sample stage, the objective lens, and the one or more image sensors.
184. The method of any one of the preceding embodiments, wherein said despeckler is configured to reduce speckle noise to be no more than 4%, 4.5%, 5%, or 5.5%.
185. The method of any one of the preceding embodiments, wherein said light source comprises a multi-color laser array.
186. The method of any one of the preceding embodiments, wherein said multi-color laser array comprises an array of laser diodes that emits laser lights at 2, 3, 4, 5, or 6 wavelengths or in 2, 3, 4, 5, or 6 wavelength ranges.
187. The method of any one of the preceding embodiments, wherein said multi-color laser array comprise lasers that emit light of 2, 3, or 4 color wavelengths or wavelength ranges at least in a direction that is orthogonal to a z axis.
188. The method of any one of the preceding embodiments, wherein said illumination subsystem further comprises one or more coupling lens.
189. The method of any one of the preceding embodiments, wherein said illumination subsystem comprises a single optical fiber.
190. The method of any one of the preceding embodiments, wherein said single optical fiber comprises a core with a maximum dimension of 500 μm to 1500 μm in a cross-section of the core.
191. The method of any one of the preceding embodiments, wherein a power in the light beam delivery subsystem is greater than 5, 8, 10, 12, or 14 Watts for one or more wavelengths or wavelength ranges.
192. The method of any one of the preceding embodiments, wherein a power at a sample plane is greater than 5, 8, 10, 12, or 14 Watts for one or more wavelengths or wavelength ranges.
193. The method of any one of the preceding embodiments, wherein said illumination subsystem further comprises a plurality of optical fibers, each optical fiber optically coupled to one or more corresponding lasers of the light source, the one or more corresponding lasers emitting light of a same wavelength or wavelength range.
194. The method of any one of the preceding embodiments, wherein said illumination subsystem further comprises one or more dichroic filters.
195. The method of any one of the preceding embodiments, wherein said light source comprises multiple light beam combiners.
196. The method of any one of the preceding embodiments, wherein said light source comprises multiple polarization light beam combiners.
197. The method of any one of the preceding embodiments, wherein said light source comprises two or more lasers that emit light at a same wavelength or in a same wavelength range.
198. The method of any one of the preceding embodiments, wherein each of said polarization light beam combiners is configured to combine light emitted from two or more lasers at the same light wavelength or in the same light wavelength range.
199. The method of any one of the preceding embodiments, wherein said despeckler is positioned in an optical path between a collimator and the sample plane.
200. The method of any one of the preceding embodiments, wherein said despeckler is positioned where a diameter of an optical beam is greater than 5 mm, 10 mm, or 20 mm, and wherein the diameter of the optical beam is orthogonal to a z-axis.
201. The method of any one of the preceding embodiments, wherein said optical fiber comprises a core with a rectangular or square cross-section.
202. The method of any one of the preceding embodiments, wherein said optical fiber comprises a core with a non-circular cross-section.
203. The method of any one of the preceding embodiments, wherein said illumination subsystem further comprises one or more liquid light guides.
204. The method of any one of the preceding embodiments, wherein said one or more liquid light guides are optically coupled to the light source in the absence of an optical fiber.
205. The method of any one of the preceding embodiments, wherein said one or more liquid light guides comprise a liquid core with a maximum dimension of 0.5 mm to 10 mm in a cross-section of the liquid core, and wherein the cross-section is orthogonal to a z-axis.
206. The method of any one of the preceding embodiments, wherein said liquid core comprises a circular cross-section.
207. The method of any one of the preceding embodiments, wherein said liquid core comprises a non-circular cross-section.
208. A sample stage for holding DNA samples for DNA sequencing reactions and imaging, comprising:
   a base stage comprising a top surface, wherein the base stage is rotatable about a z-axis relative to an optical system of a sequencing system;
   one or more top stages positioned on the top surface of the base stage, wherein each of the one or more top stages is configured to receive and secure one or more flow cell devices thereon, and wherein said each of the one or more top stages are movable relative to the base stage; a first motor configured to actuate the base stage to rotate with a first resolution.
209. The sample stage of any one of the preceding embodiments, Wherein the top surface is of a circular shape.
210. The sample stage of any one of the preceding embodiments, wherein the first resolution is angular resolution and less than 0.1 degrees, 0.2 degrees 0.5 degrees, 1 degree, 2 degrees, 3 degrees, 4 degrees, 5 degrees, 10 degrees, 20 degrees, 30 degrees, or 50 degrees.
211. The sample stage of any one of the preceding embodiments, wherein each of the flow cell devices comprises one or more samples immobilized thereon to be sequenced.
212. The sample stage of any one of the preceding embodiments, wherein at least one of the flow cell devices comprises an in situ sample immobilized thereon.
213. The sample stage of any one of the preceding embodiments, wherein the sample stage further comprises one or more second motors configured to acuate the one or more top stages relative to the base stage at a second resolution individually.
214. The sample stage of any one of the preceding embodiments, wherein the sample stage further comprises a second motor configured to acuate the one or more top stages relative to the base stage at a second resolution simultaneously.
215. The sample stage of any one of the preceding embodiments, wherein the second resolution is less than 0.01 mm, 0.015 mm, 0.02 mm, 0.03 mm, 0.04 mm, 0.05 mm, 0.08 mm, 0.1 mm, 0.2 mm, or 1 mm.
216. The sample stage of any one of the preceding embodiments, Wherein the sequencing system comprises a fluidic control device in fluidic communication with the flow cell devices positioned on the sample stage.
217. The sample stage of any one of the preceding embodiments, wherein said each of the one or more top stages are movable within a sample plane relative to the base stage.
218. The sample stage of any one of the preceding embodiments, wherein a first top stage of the one or more top stages is movable independently relative to a second top stage of the one or more top stages.
219. The sample stage of any one of the preceding embodiments, wherein a first top stage of the one or more top stages is movable simultaneously with a second top stage of the one or more top stages relative to the base stage.
220. The sample stage of any one of the preceding embodiments, wherein said each of the one or more top stages are movable along a radius of the top surface of the base stage relative to the base stage.
221. The sample stage of any one of the preceding embodiments, wherein said each of the one or more top stages are movable orthogonal to a radius of the top surface of the base stage relative to the base stage.
222. A method of sequencing multiple DNA samples positioned on a rotary sample stage, comprising:
   obtaining a sample stage comprising a base stage and one or more top stages positioned on a top surface of the base stage, wherein the base stage is rotatable about a z-axis relative to an optical system of a sequencing system;
   positioning and securing a first flow cell device relative to a first top stage of the one or more top stages;
   positioning and securing a second flow cell device relative to a second top stage of the one or more top stages;
   dispensing, by a first fluidic control device, one or more sequencing reagents to the first flow cell device;
   imaging a first sample region of the first flow cell device using the optical system of the sequencing system;
   moving the first top stage within the x-y plane relative to the optical system while preventing the second flow cell device from moving relative to the optical system;
   imaging a second sample region of the first flow cell device using the optical system of the sequencing system;
   rotating the sample stage with a predetermined angular resolution to position the second flow cell device in a predetermined position relative to the optical system; and
   imaging a first sample region of the second flow cell device using the optical system of the sequencing system.
223. The method of any one of the preceding embodiments, Wherein moving the first top stage within the x-y plane relative to the optical system while preventing the second flow cell device from moving relative to the optical system comprises:
   moving the first top stage along a radius of the top surface of the base stage with a predetermined distance relative to the optical system independently while preventing the second flow cell device from moving relative to the optical system.
224. The method of any one of the preceding embodiments, wherein moving the first top stage within the x-y plane relative to the optical system while preventing the second flow cell device from moving relative to the optical system comprises:
   moving the first top stage along a direction orthogonal to a radius of the top surface of the base stage with a predetermined distance relative to the optical system independently while preventing the second flow cell device from moving relative to the optical system.
225. The method of any one of the preceding embodiments, wherein the method further comprises: moving the first fluidic control device or a second fluidic control device to position the second fluidic cell device in a predetermined position relative to the first fluidic control device or the second fluidic control device.
226. The method of any one of the preceding embodiments, wherein the first sample region or the second sample region comprises a tile.
227. The method of any one of the preceding embodiments, wherein each of the one or more top stages comprises a motion range of greater than 15 mm and less than 80 mm, along a radius or orthogonal to the radius of the top surface of the base stage.
228. The method of any one of the preceding embodiments, wherein each of the one or more top stages comprises a motion range of greater than 25 mm and less than 100 mm, along a radius or orthogonal to the radius of the top surface of the base stage.

While preferred embodiments of the present inventive concepts have been shown and described herein, it will be obvious to those skilled in the art that such embodiments are provided by way of example only. Numerous variations, changes, and substitutions will now occur to those skilled in the art without departing from the inventive concepts. It should be understood that various alternatives to the embodiments of the inventive concepts described herein may be employed in practicing these inventive concepts. It is intended that the following claims define the scope of the inventive concepts and that methods and structures within the scope of these claims and their equivalents be covered thereby.

What is claimed is:

1. An optical system, comprising:
a stage configured to hold a solid support;
a light source configured to illuminate said solid support; and
an optical assembly disposed at least partly within an optical path from said stage to said light source, wherein said optical assembly is configured to provide an illumination over an area of said solid support that is greater than about 20 square millimeters (mm²) with a peak-to-valley variation of at most about 5%.

2. The optical system of claim 1, wherein said optical assembly does not comprise an objective.

3. The optical system of claim 2, wherein said optical system does not comprise said objective.

4. The optical system of claim 1, wherein said optical assembly does not comprise a tube lens.

5. The optical system of claim 4, wherein said optical system does not comprise said tube lens.

6. The optical system of claim 1, wherein said stage does not adjust in an optical axis of said system.

7. The optical system of claim 1, wherein said illumination has an irradiance of at least about 40 milliwatts per square millimeter.

8. The optical system of claim 1, wherein said optical assembly is configured to receive an emission light from said solid support.

9. The optical system of claim 8, wherein said optical assembly has a numerical aperture (NA) of at least about 0.3.

10. The optical system of claim 8, wherein said emission light has a wavelength of about 500 nanometers to about 750 nanometers.

11. The optical system of claim 1, wherein said optical assembly has a working distance of at least about 1 mm to 25 mm.

12. The optical system of claim 1, further comprising a motion coil housed within said optical assembly configured to move a focusing element within said optical path of said optical system.

13. The optical system of claim 1, wherein a motor external to said optical system is configured to move a focusing element along the optical axis in one or both directions.

14. The optical system of claim 13, wherein said motor is coupled directly with a piece of a first, second, or third housing of said optical assembly, and the piece of the first, second, or third housing of said optical assembly is coupled directly with said focusing element.

15. The optical system of claim 1, wherein said light source is a pulsed light source.

16. The optical system of claim 1, wherein said optical system has a composite root mean square error of less than about 0.05.

17. The optical system of claim 1, wherein said optical assembly has an illumination efficiency of at least about 90%.

18. The optical system of claim 1, wherein said area is greater than 30 mm².

19. The optical system of claim 1, wherein said area is greater than 50 mm² or 60 mm².

20. The optical system of claim 1, further comprising said solid support within said stage.

21. The optical system of claim 20, wherein said solid support comprises two or more surfaces having one or more samples immobilized thereon which are imaged by said optical system.

22. The optical system of claim 21, wherein said solid support comprises three or more surfaces having one or more samples immobilized thereon imaged by said optical system.

23. The optical system of claim 22, wherein said three or more surfaces are axially displaced from each other at least along an optical axis of said optical system.

24. The optical system of claim 20, wherein said solid support comprises a probe configured to bind a nucleic acid molecule.

25. The optical system of claim 24, wherein said probe is bound to a surface of said solid support.

26. The optical system of claim 1, wherein said light source is a laser light source.

27. The optical system of claim 1, wherein said optical assembly comprises a dichroic filter configured to transmit said illumination.

28. The optical system of claim 1, wherein said optical assembly comprises a first segment comprising a first housing comprising a first plurality of lenses, a second segment comprising a second housing, and a third segment comprising a third housing comprising a second plurality of lenses.

29. The optical system of claim 28, wherein said first segment and said third segment are optically aligned.

30. The optical system of claim 28, wherein said first segment is positioned between said third segment and said stage.

31. The optical system of claim 28, wherein said third segment is positioned between said first segment and an image sensor of the optical system.

32. The optical system of claim 28, wherein said first plurality of lenses are movable along said optical path with a range of about 0 to about 2 millimeters.

33. The optical system of claim 28, wherein said first plurality of lenses comprises an asymmetric convex-convex lens.

34. The optical system of claim 28, wherein said second plurality of lenses comprises an asymmetric concave-concave lens.

35. The optical system of claim 34, wherein said asymmetric concave-concave lens is an aspheric asymmetric concave-concave lens.

36. The optical system of claim 1, wherein said optical system is configured to acquire images of said solid support without moving an optical compensator into the optical path between said solid support and a detector of the optical system.

37. The optical system of claim 1, wherein said optical system is configured to acquire images of said solid support without moving an optical compensator out from the optical path between the sample and a detector of the optical system.

38. The optical system of claim 1, wherein said solid support is a flow cell.

39. The optical system of claim 1, wherein said optical assembly is configured to generate one or more spatial constrictions lateral to said optical path of light which travels therethrough.

40. The optical system of claim 1, wherein said optical assembly is configured to generate one or more field curvature corrections lateral to said optical path of light which travels therethrough.

41. The optical system of claim 1, wherein said optical assembly is configured to generate at least one field curvature correction lateral to the optical path of light travels therethrough in a first segment, second segment, or third segment.

42. A method of analyzing a biological molecule, comprising:
(a) providing a solid support comprising said biological molecule comprising a label;
(b) using an optical system comprising a light source to provide illumination to said biological molecule comprising said label, thereby generating a signal light or a change thereof, wherein said illumination is provided over an area of said solid support that is greater than about 20 square millimeters (mm²) with a peak-to-valley variation of at most about 5%;
(c) detecting, using a detector of said optical system, said signal light or said change thereof; and
(d) processing at least in part said signal light or said change thereof to analyze said biological molecule.

43. The method of claim 42, wherein said biological molecule is a nucleic acid molecule, a protein, or a polypeptide.

44. The method of claim 43, wherein said biological molecule is a nucleic acid.

45. The method of claim 42, further comprising, prior to (a), binding said biological molecule to a probe bound to said solid support, and coupling said label to said biological molecule.

46. The method of claim 42, wherein said label is coupled to said biological molecule by hybridization.

47. The method of claim 42, wherein said optical system does not comprise an objective.

48. The method of claim 42, wherein said solid support is not moved in an optical axis of said optical system.

49. The method of claim 48, wherein a plurality of images of said solid support are acquired without moving said solid support in said optical axis.

50. The method of claim 42, wherein said illumination has an irradiance of at least about 40 milliwatts per square millimeter.

51. The method of claim 42, wherein said signal light has a wavelength of about 500 nanometers to about 750 nanometers.

52. The method of claim 42, wherein said detecting of (c) is performed using an optical element with a numerical aperture of at least about 0.3.

53. The method of claim 42, further comprising, in (b), using a motion coil within said optical system to move a focusing element within an optical path of said optical system, thereby changing a focus of said optical system on said solid support.

54. The method of claim 42, wherein said light source is a pulsed light source.

55. The method of claim 42, wherein said illumination is provided with an efficiency of at least about 90%.

56. The method of claim 42, further comprising repeating (b)-(d) for an additional biological molecule coupled to an additional surface of said solid support.

57. The method of claim 42, further comprising, subsequent to (c), removing said label from said biological molecule.

58. The method of claim 57, further comprising repeating (a)-(d) for an additional label that binds to another portion of the biological molecule.

59. The method of claim 42, wherein an optical assembly is configured to generate one or more spatial constrictions lateral to said optical path of light which travels therethrough.

60. The method of claim 42, wherein an optical assembly is configured to generate one or more field curvature corrections lateral to said optical path of light which travels therethrough.

61. The method of claim 42, wherein an optical assembly is configured to generate at least one field curvature correction lateral to the optical path of light that travels therethrough in a first segment, second segment, or third segment.

62. The method of claim 42, wherein (d) comprises processing, at least in part, said signal light or said change thereof to generate one or more solid support images and analyze said one more solid support images to generate base calls of the sample.

63. The method of claim 62, wherein each of said solid support images comprises a field-of-view (FOV) that is greater than 20 square millimeters (mm²).

64. The method of claim 42, wherein said solid support is a flow cell.

65. An optical system, comprising:
a stage configured to hold a solid support;
a light source configured to illuminate said solid support; and
a despeckler optically coupled to said light source and disposed within an optical path from said light source to said stage.

66. The optical system of claim 65, further comprising an additional light source optically coupled into said despeckler.

67. The optical system of claim 66, wherein light from said additional light source is configured to illuminate said solid support with a different wavelength of light from said light source.

68. The optical system of claim 66, wherein at least about 4 light sources are coupled into said despeckler.

69. The optical system of claim 65, wherein said despeckler is a vibrational despeckler.

70. The optical system of claim 65, wherein said despeckler is a passive despeckler.

71. The optical system of claim 70, wherein said passive despeckler comprises a diffuse scattering plate.

72. The optical system of claim 65, wherein said despeckler is a tension despeckler.

73. The optical system of claim 65, wherein said despeckler is configured to reduce speckle noise to at most about 5%.

74. The optical system of claim 65, wherein said solid support is a flow cell.

75. A method for analyzing a biological molecule, comprising:
(a) providing a solid support comprising a biological sample comprising a label;
(b) using an optical system comprising a light source to provide illumination to said biological sample comprising said label, thereby generating a signal light or a change thereof, wherein said illumination is provided through a despeckler in an optical path of said optical system;
(c) detecting, using a detector of said optical system, said signal light or said change thereof; and
(d) processing at least in part said signal light or said change thereof to analyze said biological molecule.

76. The method of claim 75, further comprising repeating (b)-(d) for an additional biological sample coupled to an additional surface of said solid support.

77. The method of claim 75, further comprising, subsequent to (c), removing said label from said biological sample.

78. The method of claim 77, further comprising repeating (a)-(d) for an additional label that binds to said biological sample.

79. The method of claim 75, wherein said despeckler uses vibration to despeckle said illumination.

80. The method of claim 75, further comprising using an additional light source to illuminate said solid support.

81. The method of claim 80, wherein said additional light source provides a different wavelength of light to said solid support.

82. The method of claim 80, wherein said additional light source is optically coupled to said despeckler.

83. The method of claim 75, wherein said biological sample comprises a nucleic acid molecule, a protein, or a polypeptide.

84. The method of claim 83, wherein said biological sample comprises a nucleic acid.

85. The optical system of claim 1, wherein the optical assembly is disposed at least partly within an optical path from said stage to a detector of the optical system.

86. The optical system of claim 1, wherein an illumination system of the optical assembly is disposed within an optical path from said stage to a detector of the optical system.

87. A sample stage for holding DNA samples for DNA sequencing reactions and imaging, comprising:
a base stage comprising a top surface, wherein the base stage is rotatable about a z-axis relative to an optical system of a sequencing system;
one or more top stages positioned on the top surface of the base stage, wherein each of the one or more top stages are configured to receive and secure one or more flow cell devices thereon, and wherein said each of the one or more top stages are movable relative to the base stage;
a first motor configured to actuate the base stage to rotate with a first resolution.
The sample stage of any one of the preceding claims, Wherein the top surface is of a circular shape.

88. The sample stage of any one of the preceding claims, wherein the first resolution is angular resolution and less than 0.1 degrees, 0.2 degrees 0.5 degrees, 1 degree, 2 degrees, 3 degrees, 4 degrees, 5 degrees, 10 degrees, 20 degrees, 30 degrees, or 50 degrees.

89. The sample stage of any one of the preceding claims, wherein each of the flow cell devices comprises one or more samples immobilized thereon to be sequenced.

90. The sample stage of any one of the preceding claims, wherein at least one of the flow cell devices comprises an in situ sample immobilized thereon.

91. The sample stage of any one of the preceding claims, wherein the sample stage further comprises one or more second motors configured to actuate the one or more top stages relative to the base stage at a second resolution individually.

92. The sample stage of any one of the preceding claims, wherein the sample stage further comprises a second motor configured to acuate the one or more top stages relative to the base stage at a second resolution simultaneously.

93. The sample stage of any one of the preceding claims, wherein the second resolution is less than 0.01 mm, 0.015 mm, 0.02 mm, 0.03 mm, 0.04 mm, 0.05 mm, 0.08 mm, 0.1 mm, 0.2 mm, or 1 mm.

94. The sample stage of any one of the preceding claims, wherein the sequencing system comprises a fluidic control device in fluidic communication with the flow cell devices positioned on the sample stage.

95. The sample stage of any one of the preceding claims, wherein said each of the one or more top stages are movable within a sample plane relative to the base stage.

96. The sample stage of any one of the preceding claims, wherein a first top stage of the one or more top stages is movable independently relative to a second top stage of the one or more top stages.

97. The sample stage of any one of the preceding claims, wherein a first top stage of the one or more top stages is movable simultaneously with a second top stage of the one or more top stages relative to the base stage.

98. The sample stage of any one of the preceding claims, wherein said each of the one or more top stages are movable along a radius of the top surface of the base stage relative to the base stage.

99. The sample stage of any one of the preceding claims, wherein said each of the one or more top stages are movable orthogonal to a radius of the top surface of the base stage relative to the base stage.

100. A method of sequencing multiple DNA samples positioned on a rotary sample stage, comprising:
obtaining a sample stage comprising a base stage and one or more top stages positioned on a top surface of the base stage, wherein the base stage is rotatable about a z-axis relative to an optical system of a sequencing system;
positioning and securing a first flow cell device relative to a first top stage of the one or more top stages;
positioning and securing a second flow cell device relative to a second top stage of the one or more top stages;
dispensing, by a first fluidic control device, one or more sequencing reagents to the first flow cell device;
imaging a first sample region of the first flow cell device using the optical system of the sequencing system;
moving the first top stage within the x-y plane relative to the optical system while preventing the second flow cell device from moving relative to the optical system;
imaging a second sample region of the first flow cell device using the optical system of the sequencing system;
rotating the sample stage with a predetermined angular resolution to position the second flow cell device in a predetermined position relative to the optical system; and
imaging a first sample region of the second flow cell device using the optical system of the sequencing system.

101. The method of any one of the preceding claims, Wherein moving the first top stage within the x-y plane relative to the optical system while preventing the second flow cell device from moving relative to the optical system comprises:
moving the first top stage along a radius of the top surface of the base stage with a predetermined distance relative to the optical system independently while preventing the second flow cell device from moving relative to the optical system.

102. The method of any one of the preceding claims, wherein moving the first top stage within the x-y plane relative to the optical system while preventing the second flow cell device from moving relative to the optical system comprises:
moving the first top stage along a direction orthogonal to a radius of the top surface of the base stage with a predetermined distance relative to the optical system independently while preventing the second flow cell device from moving relative to the optical system.

103. The method of any one of the preceding claims, wherein the method further comprises:
moving the first fluidic control device or a second fluidic control device to position the second fluidic cell device in a predetermined position relative to the first fluidic control device or the second fluidic control device.

104. The method of any one of the preceding claims, wherein the first sample region or the second sample region comprises a tile.

105. The method of any one of the preceding claims, wherein each of the one or more top stages comprises a motion range of greater than 15 mm and less than 80 mm, along a radius or orthogonal to the radius of the top surface of the base stage.

106. The method of any one of the preceding claims, wherein each of the one or more top stages comprises a motion range of greater than 25 mm and less than 100 mm, along a radius or orthogonal to the radius of the top surface of the base stage.
